(19) United States
(12) Patent Application Publication
(10) Pub. No.: US 2005/0187172 A1
(43) Pub. Date: Aug. 25, 2005

(54) Combination of a Cox-2 inhibitor and a DNA topoisomerase I inhibitor for treatment of neoplasia

(76) Inventor: Jaime L. Masferrer, Ballwin, MO (US)

Correspondence Address: PFIZER INC, 150 EAST 42ND STREET, 5TH FLOOR - STOP 49, NEW YORK, NY, 10017-5612, US

(21) Appl. No.: 11/022,174

(22) Filed: Dec. 23, 2004

Related U.S. Application Data
(60) Provisional application No. 60/532,203, filed on Dec. 23, 2003.

(51) Int. Cl. A61K 31/7056; A61K 31/553; A61K 31/4745; A61K 31/415; A61K 31/365

(52) U.S. Cl. 514/43; 514/283; 514/406; 514/471; 514/602; 514/211.08

(57) ABSTRACT

The present invention provides combinations of a Cox-2 inhibitor and a DNA topoisomerase inhibitor and methods of use thereof for preventing and/or treating neoplasia or or a neoplasia-related disorder in a subject.

This application claims the benefit of U.S. provisional application Ser. No. 60/532,203, filed on Dec. 23, 2003, the disclosure of which in its entirety is incorporated by reference herein.

### FIELD OF THE INVENTION

The present invention relates generally to compositions and methods for the prevention or treatment of neoplasia and neoplasia-related disorders, and more particularly to the prevention or treatment of neoplasia and neoplasia-related disorders by the administration of a combination of enzyme inhibitors.

### BACKGROUND OF THE INVENTION

A neoplasm, or tumor, is an abnormal, unregulated, and disorganized proliferation of cell growth. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now the second leading cause of death in the United States and over 8,000,000 persons in the United States have been diagnosed with cancer. In 1995, cancer accounted for 23.3% of all deaths in the United States. See U.S. Dept. of Health and Human Services, National Center for Health Statistics, Health United States 1996-97 and Injury Chartbook 117 (1997).

Cancer is not fully understood on the molecular level. It is known that exposure of a cell to a carcinogen such as certain viruses, certain chemicals, or radiation, leads to alterations in DNA that can inactivate a “suppressive” gene or activate an “oncogene”. Suppressive genes are growth regulatory genes which, upon mutation, can no longer control cell growth. Oncogenes are initially normal genes (called proto-oncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality, as transformed cells can grow indefinitely.

Cancer is currently treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia. Moreover, surgical treatments are generally only successful if the cancer is detected at an early stage and before the cancer has metastasized to major organs, thus making surgery non-feasible.

Chemotherapy involves the disruption of cell replication or cell metabolism, ultimately resulting in cell death. Chemotherapeutic agents are traditionally administered via intravenous, systemic delivery, subjecting both normal and cancerous cells to their toxic effects. The adverse effects associated with systemic chemotherapy used in the treatment of neoplastic disease are problematic for patients undergoing cancer treatment. Of these adverse effects, nausea and vomiting are the most common side effects. More severe side effects include, for example, myelosuppression, which undermines the patient's ability to ward off infection and allows the spread of cancerous cells. Other adverse side effects include cytopenia, infection, cachexia, mucositis in patients receiving high doses of chemotherapy with bone marrow rescue or radiation therapy, alopecia (hair loss), cutaneous complications (See M. D. Abeloff, et al: Alopecia and Cutaneous Complications, p. 755-56, in Abeloff, M. D., Armitage, J. O., Lichter, A. S., and Niederhuber, J. E. (eds) Clinical Oncology. Churchill Livingston, New York, (1992) for cutaneous reactions to chemotherapy agents, such as pruritis, urticaria), and angioedema, neurological complications, pulmonary and cardiac complications in patients receiving radiation or chemotherapy, and reproductive and endocrine complications. Of concern is that chemotherapy induced side effects significantly impact the quality of life of the patient and may dramatically influence patient compliance with treatment.

One particularly effective class of chemotherapeutic agents includes inhibitors of DNA topoisomerases. DNA topoisomerases are enzymes that affect the structure of DNA. More specifically, these enzymes have the ability to introduce supercoils into DNA molecules or relax the DNA molecules; they can catenate or decatenate circular DNA or they can knot or unknot DNA. See Schmid et al., BioEssays 15: 445-9 (1993). The DNA topoisomerases act by catalyzing the breakage and rejoining of the DNA phosphodiester backbone. These reactions, together with an intervening strand passage event, allow topoisomerases to alter DNA topology. In fact, proper topoisomerase function is necessary for basic cellular processes such as DNA replication and transcription, and ultimately cell division.

More specifically, the DNA topoisomerases are classified into two types. Type I topoisomerases act by causing a transient break in one strand of the double-stranded DNA and passing one strand of DNA through another, thereby allowing for the relaxation of supercoiled DNA and decatenation of interlocked circular DNA molecules. See Schmid et al., supra. In contrast, Type II DNA topoisomerases alter DNA topology by causing transient breaks in both strands of a double-stranded DNA, allowing the passage of one double-stranded DNA molecule through another. Like Type I DNA topoisomerases, the Type II topoisomerases also allow the relaxation and decatenation of DNA. The mechanism of action for either type of topoisomerase, after the strand passage event, requires the final step of rejoining or ligation of the DNA break(s).

Analysis of the mechanism of action of the DNA topoisomerases on the molecular level indicates that these enzymes introduce breaks in the DNA molecule by forming a covalent phosphotyrosine bond between the phosphodiester backbone of the DNA and a specific tyrosine amino acid residue present on the topoisomerase protein. Thus, an intermediate in the catalytic reaction of these enzymes is a covalently-linked enzyme-DNA complex, sometimes termed the cleavable complex. See, e.g., Liu, DNA Topology and Its Biological Effects, Cozzarelli and Wang (eds.), Chapter 14, pp. 371-372, Cold Spring Harbor Press (1990). It is this cleavable complex that forms the molecular target for therapeutic compounds that can interact with this intermediate, stabilizing or “trapping” the complex, such that the subsequent DNA strand ligation step of the reaction cannot be completed.

Compounds that are effective cellular inhibitors of DNA topoisomerases are expected to act as cytotoxic agents through the disruption of the normal cell division process. Because cell division is an important characteristic of cancers and other proliferative diseases, agents that inhibit topoisomerases are useful as antineoplastic agents. However, cell division occurring in normal, noncancerous cells is also disrupted by these agents, leading to some of the associated side effects.

In addition to impacting patient quality of life, the adverse side effects associated with chemotherapeutic agents such as DNA topoisomerase inhibitors are generally the major dose-limiting toxicity (DLT) in the administration of these drugs. For example, mucositis is a major dose limiting toxicity for several anticancer agents, including the antimetabolite cytotoxic agents 5-FU, methotrexate, and antitumor antibiotics, such as doxorubicin. Many of these chemotherapy-induced side effects, if severe, may lead to hospitalization, or require treatment with analgesics for the treatment of pain.

Historically, physicians have treated inflammation-related disorders with a regimen of nonsteroidal anti-inflammatory drugs (NSAIDS), such as, for example, aspirin and ibuprofen. Of particular interest is the recent discovery that NSAID use has been associated with the prevention and treatment of several types of cancer. See Thun, M., et al., J. National Cancer Inst. 94(4):252-266 (2002). Undesirably, however, some NSAIDS are known to cause gastrointestinal (GI) bleeding or ulcers in patients undergoing consistent long-term regimens of NSAID therapy. See Henry, D., et al., Lancet 337:730 (1991).

It is now widely recognized that many of the traditional NSAIDs are inhibitors of two cyclooxygenases, cyclooxygenase-1 (Cox-1) and cyclooxygenase-2 (Cox-2). These two enzymes are involved in the critical initiation step of prostaglandin synthesis—the addition of molecular oxygen to arachidonic acid in the cell membrane. See Needleman, P. et al., Annu. Rev. Biochem. 55:69-102 (1986).

Cox-1 is constituitively active and is responsible for the synthesis of housekeeping prostaglandins critical to maintaining normal renal function, gastric mucosal integrity, and vascular homeostasis. Cox-2 expression is induced by cytokines and growth factors in inflammatory cells, leading to the release of prostanoids, for example, prostaglandin E2, which sensitize peripheral nociceptor terminals and produce localized pain hypersensitivity, inflammation, and edema. See e.g. Samad, T. A. et al., Nature 410:471-5 (2001). Because many common NSAIDs inhibit prostaglandin synthesis by blocking the activity of both Cox-1 and Cox-2, side effects associated with long-term administration of these drugs such as gastrointestinal bleeding and ulcers are thought to be a result of inhibiting the homeostatic functions of Cox-1, while the inhibiton of Cox-2 accounts for their analgesic and anti-inflammatory properties.

Research into the area of arachidonic acid metabolism has resulted in the discovery of compounds that inhibit the Cox-2 enzyme to a greater extent than the activity of Cox-1. The Cox-2-selective inhibitors are believed to offer advantages that include the capacity to prevent or reduce inflammation while avoiding harmful side effects associated with the inhibition of Cox-1. Thus, Cox-2 selective inhibitors have shown great promise for use in therapies, especially in therapies that require long term use.

Of particular importance for the present invention is that overexpression of Cox-2 has been documented in several premalignant and maliganant tissues. See Subbaramaiah, K. and Dannenberg, A. J. Trends Pharmacol Sci, 24:96-102 (2003). This increase in expression is thought to be a product of induction of protein kinase C (PKC) signaling, which stimulates the activity of mitogen-activated protein kinase (MAPK), enhancing transcription of Cox-2 by nuclear factors. Additionally, enhanced stability of Cox-2 mRNA transcripts in cancer cells due to augmented binding of the RNA-binding protein HuR, as well as activation of extracellular signal related kinase 1/2 (ERK 1/2) and p38, contributes to increased expression of Cox-2. Id.

Adverse side effects induced by anticancer therapy have become of major importance in the clinical management of patients undergoing treatment for cancer or neoplasia disease. Therefore, there is a need for improved methods and compositions for the prevention and treatment of neoplasia and neoplasia-related disorders that treat more than one aspect of neoplasia and neoplasia-related disorders and that are efficacious for such applications in physiologically acceptable dosages, and which are selective in their physiological impact.

Combination therapies for treatment of neoplasia including a Cox-2 selective inhibitor and a DNA topoisomerase I inhibitor, for example irinotecan or its hydrochloride salt (CPT-11), are disclosed in the publications individually cited below and incorporated herein by reference in their entirety.

U.S. Pat. No. 5,972,986.

International Patent Publication No. WO 00/38730.

International Patent Publication No. WO 02/85459.

### SUMMARY OF THE INVENTION

The present invention is directed to a method of preventing or treating neoplasia or a neoplasia-related disorder in a subject, the method comprising administering in combination therapy to a subject a Cox-2 inhibitor and a DNA topoisomerase I inhibitor.

In one embodiment the method comprises administering in combination therapy to the subject a Cox-2 selective inhibitor and a DNA topoisomerase I inhibitor selected from the group consisting of camptothecin in combination with poly-(l-glutamic acid), camptothecin in combination with NU1025, XR-11612, DX-8915f, anthracycline aclacinomycin A, harmane, harmine, harmaline, bulgarein, rebeccamycin, rebeccamycin R-3, luteolin, diospyrin, ecteinascidin 743, Ho-33342, Ho-33258, idarubicin, SN-38 in combination with 5-FU in sequential drug administration with SN-38 first, 9-NC in combination with 5-FU, BN-80927, fagaronine, ethoxidine, nitidine, MJ-III-65, S2, J-107088, karenitecin, BNP-1100 in combination with ZD-1694, β-lapachone, intoplicine, TAN-1518A, plaquiloside, GI-147211, camptothecin in combination with 7-hydroxystaurosporine, indeneisoquinolines, heteroaromatic[a]phenazine carboxamide derivatives, covalent conjugates of topoisomerase I and topoisomerase II inhibitors, 7-substituted camptothecin derivatives, highly lipophilic camptothecin derivatives, hexacyclic camptothecin analogues, trisbenzimidazoles, benzo[a]phenazine-11-carboxamide derivatives, XR-5000, phenoxodiol, AHMA, (5Z,9Z)-5,9-hexadecadienoic acid, RFS2000, TAS-103, 7-ethyl-10-[4-(1-piperdyl)-1-piperidyl]carbonyloxy-camptothecin, disulfiram, isoaurostatin, 6-[3-(2-hydroxyethyl)aminopropyl]-5,6-dihydro-2,3-dimethoxy-8,9-methylenedioxy-5,11-dioxo-11H-indeno [1,2-c]isoquinoline hydrochloride, BNP1350 and ring-substituted 11-oxo-1H-indeno[1,2-b]quinoline-6-carboxamides.

In another embodiment the method comprises administering in combination therapy to the subject a Cox-2 selective inhibitor and a DNA topoisomerase I inhibitor, wherein the Cox-2 selective inhibitor comprises at least one compound of formulas (XXXVII) to (LI) hereinbelow.

The present invention is also directed to a novel therapeutic composition comprising at least one Cox-2 inhibitor and one or more DNA topoisomerase I inhibitors.

The present invention is also directed to a novel pharmaceutical composition comprising a Cox-2 inhibitor, a DNA topoisomerase I inhibitor, and a pharmaceutically acceptable carrier.

The present invention is also directed to a novel kit for preventing and treating neoplasia and neoplasia-related disorders in a subject, the kit comprising one dosage form comprising a Cox-2 inhibitor and a second dosage form comprising a DNA topoisomerase I inhibitor.

Several advantages are achieved by the present invention, including the provision of methods and compositions that are effective for the prevention and treatment of neoplasia and neoplasia-related disorders. Also provided by the present invention are methods and compositions that treat more than one aspect of neoplasia and neoplasia-related disorders and that are efficacious for such applications in physiologically acceptable dosages, and which are selective in their physiological impact. Finally, the present invention provides improved methods and compositions for preventing and treating neoplasia and neoplasia-related disorders.

### DETAILED DESCRIPTION

In accordance with the present invention, it has been discovered that neoplasia and neoplasia-related disorders may be treated and prevented in a subject by administering to the subject a Cox-2 inhibitor in combination with a DNA topoisomerase I inhibitor.

For purposes of the present invention, the novel combination therapy comprising at least one Cox-2 inhibitor in combination with at least one DNA topoisomerase I inhibitor is useful for the purpose of preventing and treating neoplasia and neoplasia-related disorders in a subject.

In preferred embodiments, the subject is one that is in need of the prevention or treatment of a neoplasia or neoplasia-related disorder.

Thus, the combination therapy of the present invention would be useful, for example, to reduce neoplasia disorder symptoms such as, for example; 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents. The combination therapy of the present invention would also be useful to prevent the occurrence of such symptoms.

In preferred embodiments, the methods and compositions of the present invention are also useful to reduce the number of hospitalizations of subjects suffering from a neoplasia and neoplasia-related disorders, or to prevent or retard, in subjects, the development of complications associated with neoplasia, which may eventually arise from having a neoplasia and neoplasia-related disorders.

The administration of a Cox-2 inhibitor alone or in combination with a DNA topoisomerase I inhibitor for the prevention and treatment of neoplasia and neoplasia-related disorders is an unexpectedly effective treatment and preventative therapy. Such administration is effective for improving the symptoms of neoplasia and neoplasia-related disorders while avoiding or reducing certain disadvantages of current treatments.

The combination therapy of a Cox-2 inhibitor and a DNA topoisomerase I inhibitor is also useful for decreasing the required number of separate dosages, thus, potentially improving patient compliance. For example, in one embodiment, the combination therapy of the present invention is useful for reducing the dosing frequency of conventional neoplasia treatment agents. Thus, administering the combination therapy of the present invention to a subject undergoing multiple dosing with an anti-neoplasia agent may reduce the required number of separate doses normally prescribed.

Combination therapies comprising Cox-2 inhibitors and DNA topoisomerase I inhibitors are useful not only for improving neoplasia and neoplasia-related disorders symptoms and shortening recovery times, but also for reducing the dosages of conventional neoplasia treatment agents that are normally required.

For example, in preferred embodiments, the combination therapy is effective for lowering the dosages of conventional neoplasia treatment agents and/or DNA topoisomerase I inhibitors that are normally prescribed as a monotherapy. The administration of lower dosages of conventional treatment agents provides a reduction in side effects corresponding to such conventional agents. Reduced dosages of conventional neoplasia treatment agents are beneficial where normal dosages often exhibit harmful side effects.

The administration of a Cox-2 inhibitor in combination with a DNA topoisomerase I inhibitor is an effective treatment for neoplasia and neoplasia-related disorders, and in preferred embodiments, is superior to the use of either agent alone.

Moreover, in preferred embodiments, the combination therapy demonstrates a synergistic efficacy for treating and preventing neoplasia and neoplasia-related disorders that is greater than what would be expected from simply combining the two therapies.

The term “synergistic” refers to the combination of a Cox-2 inhibitor and a DNA topoisomerase I inhibitor as a combined therapy having an efficacy for the prevention and treatment of neoplasia and neoplasia-related disorders that is greater than what would be expected merely from the sum of their individual effects.

The synergistic effects of the embodiments of the present invention's combination therapy encompass additional unexpected advantages for the treatment and prevention of neoplasia and neoplasia-related disorders. Such additional advantages optionally include, but are not limited to, lowering the required dose of neoplasia treatment agents, reducing the side effects of neoplasia treatment agents, and rendering those agents more tolerable to subjects in need of neoplasia treatment.

As used herein, the phrases “combination therapy”, “co-administration”, “co-administering”, “administration with”, “administering”, “combination”, or “co-therapy”, when referring to use of a Cox-2 inhibitor in combination with a DNA topoisomerase I inhibitor, are intended to embrace administration of each agent in a sequential manner in a regimen that will provide beneficial effects of the drug combination, and is intended as well to embrace co-administration of these agents in a substantially simultaneous manner. Thus, the Cox-2 inhibitor and DNA topoisomerase I inhibitor may be administered in one therapeutic dosage form, such as in a single capsule, tablet, or injection, or in two separate therapeutic dosage forms, such as in separate capsules, tablets, or injections.

Sequential administration of such treatments encompasses both relatively short and relatively long periods between the administration of each of the drugs of the present method. However, for purposes of the present invention, the second drug is administered while the first drug is still having an efficacious effect on the subject. Thus, the present invention, in one embodiment, takes advantage of the fact that the simultaneous presence of the combination of a Cox-2 inhibitor and a DNA topoisomerase I inhibitor in a subject has a greater efficacy than the administration of either agent alone.

Preferably, the second of the two drugs is given to the subject within the therapeutic response time of the first drug to be administered. For example, the present invention encompasses administration of a Cox-2 inhibitor to the subject and the later administration of a DNA topoisomerase I inhibitor, as long as the DNA topoisomerase I inhibitor is administered to the subject while the Cox-2 inhibitor is still present in the subject at a level, which in combination with the level of the DNA topoisomerase I inhibitor, is therapeutically effective, and vice versa.

As used herein, the term “therapeutic response time” means the duration of time that a compound is present or detectable within a subject's body at therapeutic concentrations.

In one embodiment, the present invention encompasses a method for preventing a neoplasia or neoplasia-related disorder in a subject, the method comprising administering to the subject a DNA topoisomerase I inhibitor and a Cox-2 inhibitor.

As used herein, the terms “to prevent”, “preventing”, or “prevention” refer to any reduction, no matter how slight, of a subject's predisposition or risk for developing a neoplasia or neoplasia-related disorder. For purposes of prevention, the subject is any subject, and preferably is a subject that is at risk for, or is predisposed to, developing a neoplasia or neoplasia-related disorder or a neoplasia-related complication. The term “prevention” includes either preventing the onset of clinically evident neoplasia altogether or preventing the onset of a preclinically evident stage of neoplasia in individuals at risk. Also intended to be encompassed by this definition is the prevention of initiation for malignant cells or to arrest or reverse the progression of premalignant cells to malignant cells. This includes prophylactic treatment of those at risk of developing the neoplasia.

As used herein, a subject that is “predisposed to” or “at risk for,” used interchangeably herein, includes any subject with an increased chance for developing a neoplasia-related disorder or any neoplasia-related complication. The subject may be at risk due to genetic predisposition, diet, age, exposure to neoplasia causing agents, and the like. The subject may also be at risk for re-developing neoplasia during a relapse of such a disorder. For example, after treatment, many neoplasia disorders subside into remission, meaning that the disease is present, but inactive within the subject and is thus, capable of re-developing at a later time. The subject may also be at risk due to physiological factors such as anatomical and biochemical abnormalities and certain autoimmune diseases.

In another embodiment, the present invention encompasses a method for treating a neoplasia or neoplasia related disorder in a subject, the method comprising administering to the subject a DNA topoisomerase I inhibitor and a Cox-2 inhibitor.

The terms “treating” or “to treat” mean to alleviate symptoms, eliminate the causation of symptoms, either on a temporary or permanent basis, or to alter or slow the appearance of symptoms. The term “treatment” includes alleviation of, elimination of causation of, or inhibition of symptoms associated with any of the diseases or disorders described herein.

The term “inhibition” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The present invention is directed to a novel method of preventing or treating neoplasia and neoplasia-related disorders in a subject comprising administering to the subject a Cox-2 inhibitor in combination with a DNA topoisomerase I inhibitor.

A component of the present invention is a Cox-2 inhibitor. The terms “cyclooxygenase-2 inhibitor”, or “Cox-2 inhibitor”, which can be used interchangeably herein, embrace compounds which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, 4-(nitrooxy)butyl ester.

In a preferred embodiment, the Cox-2 inhibitor is a Cox-2 selective inhibitor. The term “Cox-2 selective inhibitor” embraces compounds which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound required to produce 50% inhibition of cyclooxygenase activity. Preferred Cox-2 selective inhibitors of the invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2 selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

As used herein, the term “alkyl”, either alone or within other terms such as “haloalkyl” and “alkylsulfonyl”, embraces linear or branched radicals having one to about twenty carbon atoms. Lower alkyl radicals have one to about ten carbon atoms. The number of carbon atoms can also be expressed as “C₁-C₅”, for example. Examples of lower alkyl radicals include methyl, ethyl, n-propyl, isopropyl, n-butyl, isobutyl, sec-butyl, tert-butyl, pentyl, isoamyl, hexyl, octyl and the like.

The term “alkenyl” refers to an unsaturated, acyclic hydrocarbon radical, linear or branched, containing at least one double bond. Alkenyl radicals may be optionally substituted with groups such as those defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like.

The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups such as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-3-yl, 3,3-dimethylbutyn-1-yl radicals, and the like.

The term “oxo” means a single double-bonded oxygen.

The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo alkyl radicals may have two or more of the same halo atoms or a combination of different halo radicals and polyhaloalkyl radicals may have more than two of the same halo atoms or a combination of different halo radicals.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and diaikoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy.

The term “aryl”, whether used alone or with other terms, means a carbocyclic aromatic system containing one, two, or three rings wherein such rings may be attached together in a pendent manner, or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms are replaced by N, S, P, or O. This includes, for example, structures such as 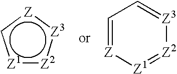
where Z, Z¹, Z² or Z³ is C, S, P, O or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others.

The term “heteroaryl” embraces unsaturated heterocyclic radicals. Examples of unsaturated heterocyclic radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like.

The term “sulfonyl”, used alone or in linked terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The term “aminosulfonyl” denotes a sulfonyl radical substituted with an amine radical, forming a sulfonamide (—SO₂—NH₂).

The terms “carboxy” or “carboxyl”, whether used alone or with other terms, such as “carboxyalkyl”, denote —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “amino”, whether used alone or with other terms, such as “aminocarbonyl”, denotes —NH₂.

The term “heterocycloalkyl” embraces heterocyclic-substituted alkyl radicals such as pyridylmethyl and thienylmethyl. The terms “aralkyl”, or “arylalkyl” embrace aryl-substituted alkyl radicals such as benzyl, diphenylmethyl, triphenylmethyl, phenylethyl, and diphenylethyl. The terms benzyl and phenylmethyl are inter-changeable. The term “cycloalkyl” embraces radicals having 3-10 carbon atoms, such as cyclopropyl, cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl.

The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The term “acyl”, whether used alone, or within a term such as “acylamino”, denotes a radical provided by the residue after removal of hydroxyl from an organic acid.

The term “cyano”, used either alone or with other terms, such as “cyanoalkyl”, refers to C≡N. The term “nitro” denotes —NO₂.

In one embodiment the Cox-2 selective inhibitor is meloxicam or a pharmaceutically acceptable salt or prodrug thereof.

In another embodiment the Cox-2 selective inhibitor is RS 57067 (6-[[5-(4-chlorobenzoyl)-1,4-dimethyl-1H-pyrrol-2-yl]methyl]-3(2H)-pyridazinone) or a pharmaceutically acceptable salt or prodrug thereof.

In another embodiment the Cox-2 selective inhibitor is of the chromene or chroman structural class that is a substituted benzopyran or a substituted benzopyran analog, for example selected from the group consisting of substituted benzothiopyrans, dihydroquinolines and dihydronaphthalenes. These compounds can have a structure as shown in any of formulas (I), (II), (III), (IV), (V) and (VI) below, and as illustrated in Table 1, and can be diastereomers, enantiomers, racemates, tautomers, salts, esters, amides and prodrugs of such compounds.

Benzopyrans that can serve as a COX-2 selective inhibitor of the present invention include substituted benzopyran derivatives that are described in U.S. Pat. No. 6,271,253, incorporated herein by reference. One such class of compounds is defined by the general formula shown below in formula (I): 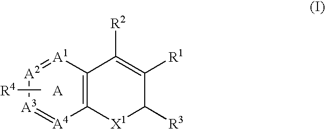
wherein:
X¹ is selected from O, S, CR^{c}R^{b} and NR^{a}, where R^{a} is selected from hydrido, C₁-C₃ alkyl, (optionally substituted phenyl)-C₁-C₃ alkyl, acyl and carboxy-C₁-C₆ alkyl; and where each of R^{b} and R^{c} is independently selected from hydrido, C₁-C₃ alkyl, phenyl-C₁-C₃ alkyl, C₁-C₃ perfluoroalkyl, chloro, C₁-C₆ alkylthio, C₁-C₆ alkoxy, nitro, cyano and cyano-C₁-C₃ alkyl; or where CR^{b}R^{c} forms a 3-6 membered cycloalkyl ring;
R¹ is selected from carboxyl, aminocarbonyl, C₁-C₆ alkylsulfonylaminocarbonyl and C₁-C₆ alkoxycarbonyl;
R² is selected from hydrido, phenyl, thienyl, C₁-C₆ alkyl and C₂-C₆ alkenyl;
R³ is selected from C₁-C₃ perfluoroalkyl, chloro, C₁-C₆ alkylthio, C₁-C₆ alkoxy, nitro, cyano and cyano-C₁-C₃ alkyl;
R⁴ is one or more radicals independently selected from hydrido, halo, C₁-C₆ alkyl, C₂-C₆ alkenyl, C₂-C₆ alkynyl, halo-C₂-C₆ alkynyl, aryl-C₁-C₃ alkyl, aryl-C₂-C₆ alkynyl, aryl-C₂-C₆ alkenyl, C₁-C₆ alkoxy, methylenedioxy, C₁-C₆ alkylthio, C₁-C₆ alkylsulfinyl, aryloxy, arylthio, arylsulfinyl, heteroaryloxy, C₁-C₆ alkoxy-C₁-C₆ alkyl, aryl-C₁-C₆ alkyloxy, heteroaryl-C₁-C₆ alkyloxy, aryl-C₁-C₆ alkoxy-C₁-C₆ alkyl, C₁-C₆ haloalkyl, C₁-C₆ haloalkoxy, C₁-C₆ haloalkylthio, C₁-C₆ haloalkylsulfinyl, C₁-C₆ haloalkylsulfonyl, C₁-C₃ haloalkyl-C₁-C₃ hydroxyalkyl, C₁-C₆ hydroxyalkyl, hydroxyimino-C₁-C₆ alkyl, C₁-C₆ alkylamino, arylamino, aryl-C₁-C₆ alkylamino, heteroarylamino, heteroaryl-C₁-C₆ alkylamino, nitro, cyano, amino, aminosulfonyl, C₁-C₆ alkylaminosulfonyl, arylaminosulfonyl, heteroarylaminosulfonyl, aryl-C₁-C₆ alkylaminosulfonyl, heteroaryl-C₁-C₆ alkylaminosulfonyl, heterocyclyl-sulfonyl, C₁-C₆ alkylsulfonyl, aryl-C₁-C₆ alkylsulfonyl, optionally substituted aryl, optionally substituted heteroaryl, aryl-C₁-C₆ alkylcarbonyl, heteroaryl-C₁-C₆ alkylcarbonyl, heteroarylcarbonyl, arylcarbonyl, aminocarbonyl, C₁-C₁ alkoxycarbonyl, formyl, C₁-C₆ haloalkylcarbonyl and C₁-C₆ alkylcarbonyl; and
the A ring atoms A¹, A², A³ and A⁴ are independently selected from carbon and nitrogen with the proviso that at least two of A¹, A², A³ and A⁴ are carbon; or
R⁴ together with ring A forms a radical selected from naphthyl, quinolinyl, isoquinolinyl, quinolizinyl, quinoxalinyl and dibenzofuryl;
   or an isomer or pharmaceutically acceptable salt thereof.

Another class of benzopyran derivatives that can serve as the COX-2 selective inhibitor of the present invention includes a compound having the structure of formula (II): 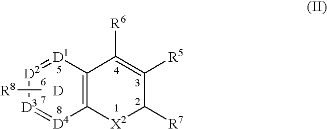
wherein:
X² is selected from O, S, CR^{c}R^{b} and NR^{a}; where R^{a} is selected from hydrido, C₁-C₃ alkyl, (optionally substituted phenyl)-C₁-C₃ alkyl, alkylsulfonyl, phenylsulfonyl, benzylsulfonyl, acyl and carboxy-C₁-C₆ alkyl; and where each of R^{b} and R^{c} is independently selected from hydrido, C₁-C₃ alkyl, phenyl-C₁-C₃ alkyl, C₁-C₃ perfluoroalkyl, chloro, C₁-C₆ alkylthio, C₁-C₆ alkoxy, nitro, cyano and cyano-C₁-C₃ alkyl;
or where CR^{c}R^{b} form a cyclopropyl ring;
R⁵ is selected from carboxyl, aminocarbonyl, C₁-C₆ alkylsulfonylaminocarbonyl and C₁-C₆ alkoxycarbonyl;
R⁶ is selected from hydrido, phenyl, thienyl, C₂-C₆ alkynyl and C₂-C₆ alkenyl;

R⁷ is selected from C₁-C₃ perfluoroalkyl, chloro, C₁-C₆ alkylthio, C₁-C₆ alkoxy, nitro, cyano and cyano-C₁-C₃ alkyl;
R⁸ is one or more radicals independently selected from hydrido, halo, C₁-C₆ alkyl, C₂-C₆ alkenyl, C₂-C₆ alkynyl, halo-C₂-C₆ alkynyl, aryl-C₁-C₃ alkyl, aryl-C₂-C₆ alkynyl, aryl-C₂-C₆ alkenyl, C₁-C₆ alkoxy, methylenedioxy, C₁-C₆ alkylthio, C₁-C₆ alkylsulfinyl, —O(CF₂)₂O—, aryloxy, arylthio, arylsulfinyl, heteroaryloxy, C₁-C₆ alkoxy-C₁-C₆ alkyl, aryl-C₁-C₆ alkyloxy, heteroaryl-C₁-C₆ alkyloxy, aryl-C₁-C₆ alkoxy-C₁-C₆ alkyl, C₁-C₆ haloalkyl, C₁-C₆ haloalkoxy, C₁-C₆ haloalkylthio, C₁-C₆ haloalkylsulfinyl, C₁-C₆ haloalkylsulfonyl, C₁-C₃ haloalkyl-C₁-C₃ hydroxyalkyl, C₁-C₆ hydroxyalkyl, hydroxyimino-C₁-C₆ alkyl, C₁-C₆ alkylamino, arylamino, aryl-C₁-C₆ alkylamino, heteroarylamino, heteroaryl-C₁-C₆ alkylamino, nitro, cyano, amino, aminosulfonyl, C₁-C₆ alkylaminosulfonyl, arylaminosulfonyl, heteroarylaminosulfonyl, aryl-C₁-C₆ alkylaminosulfonyl, heteroaryl-C₁-C₆ alkylaminosulfonyl, heterocyclylsulfonyl, C₁-C₆ alkylsulfonyl, aryl-C₁-C₆ alkylsulfonyl, optionally substituted aryl, optionally substituted heteroaryl, aryl-C₁-C₆ alkylcarbonyl, heteroaryl-C₁-C₆ alkylcarbonyl, heteroarylcarbonyl, arylcarbonyl, aminocarbonyl, C₁-C₆ alkoxycarbonyl, formyl, C₁-C₆ haloalkylcarbonyl and C₁-C₆ alkylcarbonyl; and
the D ring atoms D¹, D², D³ and D⁴ are independently selected from carbon and nitrogen with the proviso that at least two of D¹, D², D³ and D⁴ are carbon; or
R⁸ together with ring D forms a radical selected from naphthyl, quinolinyl, isoquinolinyl, quinolizinyl, quinoxalinyl and dibenzofuryl;
   or an isomer or pharmaceutically acceptable salt thereof.

Other benzopyran COX-2 selective inhibitors useful in the practice of the present invention are described in U.S. Pat. Nos. 6,034,256 and 6,077,850, incorporated herein by reference. The general formula for these compounds is shown in formula (III): 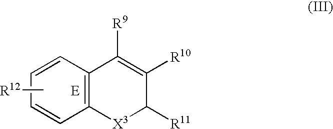
wherein:
X³ is selected from the group consisting of O or S or NR^{a} where R^{a} is alkyl;
R⁹ is selected from the group consisting of H and aryl;
R¹⁰ is selected from the group consisting of carboxyl, aminocarbonyl, alkylsulfonylaminocarbonyl and alkoxycarbonyl;
R¹¹ is selected from the group consisting of haloalkyl, alkyl, aralkyl, cycloalkyl and aryl optionally substituted with one or more radicals selected from alkylthio, nitro and alkylsulfonyl; and
R¹² is selected from the group consisting of one or more radicals selected from H, halo, alkyl, aralkyl, alkoxy, aryloxy, heteroaryloxy, aralkyloxy, heteroaralkyl-oxy, haloalkyl, haloalkoxy, alkylamino, arylamino, aralkylamino, heteroaryl-amino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyaryl-carbonyl, nitroaryl, optionally substituted aryl, optionally substituted heteroaryl, aralkylcarbonyl, heteroarylcarbonyl, arylcarbonyl, aminocarbonyl, and alkylcarbonyl; or
R¹² together with ring E forms a naphthyl radical;
   or an isomer or pharmaceutically acceptable salt thereof; and including diastereomers, enantiomers, racemates, tautomers, salts, esters, amides and prodrugs thereof.

A related class of compounds useful as COX-2 selective inhibitors in the present invention is described by formulas (IV) and (V): 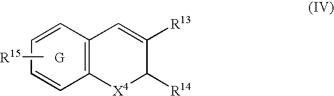
wherein:
X⁴ is selected from O or S or NR^{a} where R^{a} is alkyl;
R¹³ is selected from carboxyl, aminocarbonyl, alkylsulfonylaminocarbonyl and alkoxycarbonyl;
R¹⁴ is selected from haloalkyl, alkyl, aralkyl, cycloalkyl and aryl optionally substituted with one or more radicals selected from alkylthio, nitro and alkylsulfonyl; and
R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or
R¹⁵ together with ring G forms a naphthyl radical;
   or an isomer or pharmaceutically acceptable salt thereof.

Formula (V) is: 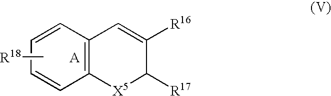
wherein:
X⁵ is selected from the group consisting of O or S or NR^{b} where R^{b} is alkyl;
R¹⁶ is selected from the group consisting of carboxyl, aminocarbonyl, alkylsulfonylaminocarbonyl and alkoxycarbonyl;
R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, aryl-aminosulfonyl, heteroarylaminosulfonyl, aralkylaminosulfonyl, heteroaralkyl-aminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl and alkylcarbonyl; or
wherein R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of lower haloalkyl, lower cycloalkyl and phenyl; and
R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkyl-aminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is carboxyl;
R¹⁷ is lower haloalkyl; and
R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylamino-sulfonyl, 6-membered heteroarylalkylaminosulfonyl, lower aralkylamino-sulfonyl, lower alkylsulfonyl, 6-membered nitrogen-containing heterocyclo-sulfonyl, optionally substituted phenyl, lower aralkylcarbonyl, and lower alkylcarbonyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl and trifluoromethyl; and
R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethyl-amino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropyl-carbonyl, phenylacetyl and phenyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting trifluoromethyl and pentafluoroethyl; and
R¹⁸ is one or more radicals selected from the group consisting of hydrido, chloro, fluoro, bromo, iodo, methyl, ethyl, isopropyl, tert-butyl, methoxy, trifluoro-methyl, trifluoromethoxy, N-phenylmethylaminosulfonyl, N-phenylethyl-aminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methyl-sulfonyl, benzylcarbonyl and phenyl; or
R¹⁸ together with ring A forms a naphthyl radical;
   or an isomer or prodrug thereof.

Another class of benzopyran derivatives that can serve as the COX-2 selective inhibitor of the present invention includes a compound having the structure of formula (VI): 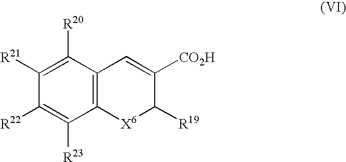
wherein:
X⁶ is selected from the group consisting of O and S;
R¹⁹ is lower haloalkyl;
R²⁰ is selected from the group consisting of hydrido and halo;
R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkyl-aminosulfonyl, 5-membered nitrogen-containing heterocyclosulfonyl, and 6-membered nitrogen-containing heterocyclosulfonyl;
R²² is selected from the group consisting of hydrido, lower alkyl, halo, lower alkoxy and aryl; and
R²³ is selected from the group consisting of the group consisting of hydrido, halo, lower alkyl, lower alkoxy, and aryl;
   or an isomer or prodrug thereof.

The COX-2 selective inhibitor can be a compound of Formula (VI), wherein:
X⁶ is selected from the group consisting of O and S;
R¹⁹ is selected from the group consisting of trifluoromethyl and pentafluoroethyl;
R²⁰ is selected from the group consisting of hydrido, chloro and fluoro;
R²¹ is selected from the group consisting of hydrido, chloro, bromo, fluoro, iodo, methyl, tert-butyl, trifluoromethoxy, methoxy, benzylcarbonyl, dimethyl-aminosulfonyl, isopropylaminosulfonyl, methylaminosulfonyl, benzylamino-sulfonyl, phenylethylaminosulfonyl, methylpropylaminosulfonyl, methyl-sulfonyl and morpholinosulfonyl;
R²² is selected from the group consisting of hydrido, methyl, ethyl, isopropyl, tert-butyl, chloro, methoxy, diethylamino and phenyl; and
R²³ is selected from the group consisting of hydrido, chloro, bromo, fluoro, methyl, ethyl, tert-butyl, methoxy and phenyl;

or an isomer or prodrug thereof.

TABLE 1
Examples of chromene Cox-2 selective inhibitors

| No. | Structural formula and name |
|---|---|
| B-3 | 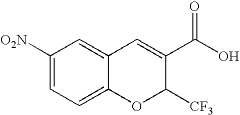 |
| | 6-Nitro-2-trifluoromethyl-2H-1- |
| | benzopyran-3-carboxylic acid |
| B-4 | 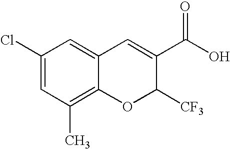 |
| | 6-Chloro-8-methyl-2-trifluoromethyl- |
| | 2H-1-benzopyran-3-carboxylic acid |
| B-5 | 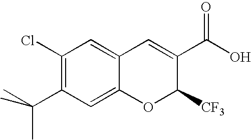 |
| | ((S)-6-Chloro-7-(1,1-dimethylethyl)-2-(tri- |
| | fluoromethyl-2H-1-benzopyran-3-carboxylic acid |
| B-6 | 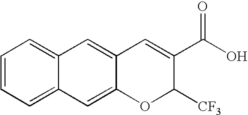 |
| | 2-Trifluoromethyl-2H-naphtho[2,3- |
| | b]pyran-3-carboxylic acid |
| B-7 | 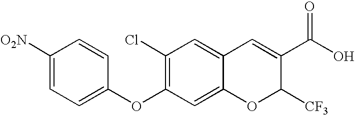 |
| | 6-Chloro-7-(4-nitrophenoxy)-2-(trifluoromethyl)-2H- |
| | 1-benzopyran-3-carboxylic acid |
| B-8 | 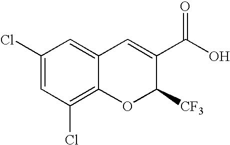 |
| | ((S)-6,8-Dichloro-2-(trifluoromethyl)- |
| | 2H-1-benzopyran-3-carboxylic acid |
| B-9 | 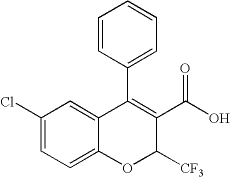 |
| | 6-Chloro-2-(trifluoromethyl)-4-phenyl-2H- |
| | 1-benzopyran-3-carboxylic acid |
| B-10 | 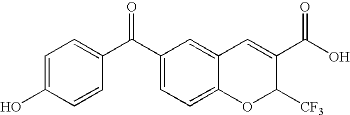 |
| | 6-(4-Hydroxybenzoyl)-2-(trifluoromethyl)- |
| | 2H-1-benzopyran-3-carboxylic acid |
| B-11 | 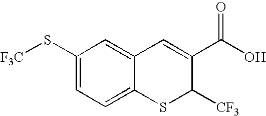 |
| | 2-(Trifluoromethyl)-6-[(trifluoromethyl)thio]- |
| | 2H-1-benzothiopyran-3-carboxylic acid |
| B-12 | 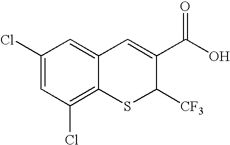 |
| | 6,8-Dichloro-2-trifluoromethyl-2H-1- |
| | benzothiopyran-3-carboxylic acid |
| B-13 | 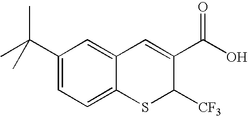 |
| | 6-(1,1-Dimethylethyl)-2-(trifluoromethyl)- |
| | 2H-1-benzothiopyran-3-carboxylic acid |
| B-14 | 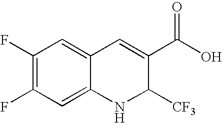 |
| | 6,7-Difluoro-1,2-dihydro-2-(trifluoro |
| | methyl)-3-quinolinecarboxylic acid |
| B-15 | 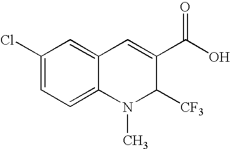 |
| | 6-Chloro-1,2-dihydro-1-methyl-2-(trifluoro |
| | methyl)-3-quinolinecarboxylic acid |
| B-16 | 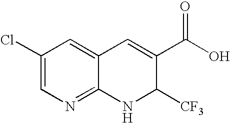 |
| | 6-Chloro-2-(trifluoromethyl)-1,2-dihydro |
| | [1,8]naphthyridine-3-carboxylic acid |
| B-17 | 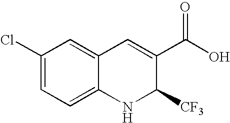 |
| | ((S)-6-Chloro-1,2-dihydro-2-(trifluoro |
| | methyl)-3-quinolinecarboxylic acid |

In other embodiments the COX-2 selective inhibitor can be selected from the class of tricyclic COX-2 selective inhibitors represented by the general structure of formula (VII): 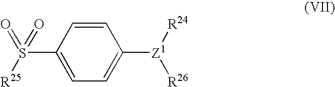
wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl and amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxy-aralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkyl-amino, N-alkyl-N-aralkylamino, N-alkyl-N-arylamino, aminoalkyl, alkylaminoalkyl, N-arylaminoalkyl, N-aralkylaminoalkyl, N-alkyl-N-aralkyl-aminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl;
   and pharmaceutically acceptable salts and prodrugs thereof.

The COX-2 selective inhibitor of formula (VII) can be selected from the group of compounds illustrated in Table 2, which includes celecoxib (B-18), valdecoxib (B-19), deracoxib (B-20), rofecoxib (B-21), etoricoxib or MK-663 (B-22) and JTE-522 (B-23), and pharmaceutically acceptable salts and prodrugs thereof.

Additional information about these COX-2 selective inhibitors can be found in patents individually cited below and incorporated herein by reference.

U.S. Pat. No. 5,466,823.

U.S. Pat. No. 5,840,924.

International Patent Publication No. WO 00/25779.

International Patent Publication No. WO 98/03484.

TABLE 2
Examples of tricyclic Cox-2 selective inhibitors

| No. | Structural formula |
|---|---|
| B-18 | 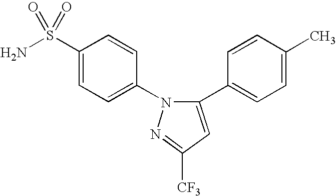 |
| B-19 | 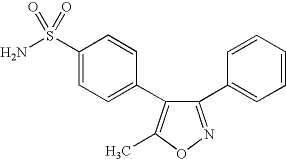 |
| B-20 | 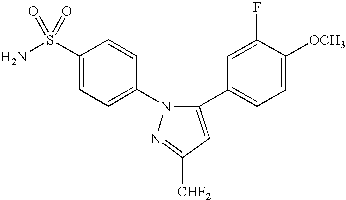 |
| B-21 | 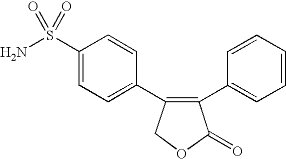 |
| B-22 | 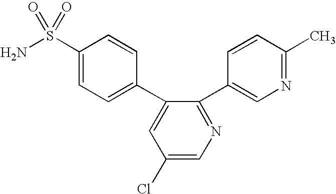 |
| B-23 | 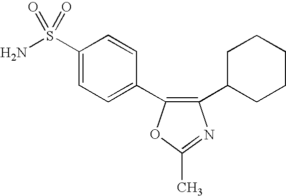 |

In certain embodiments of the invention, the Cox-2 selective inhibitor is selected from the group consisting of celecoxib, rofecoxib and etoricoxib.

In one embodiment of the invention, parecoxib (see, e.g., U.S. Pat. No. 5,932,598), which is a therapeutically effective prodrug of the tricyclic Cox-2 selective inhibitor valdecoxib, B-19 (see, e.g., U.S. Pat. No. 5,633,272), may be advantageously employed as a source of a Cox-2 inhibitor.

Parecoxib can be used as a salt, for example parecoxib sodium.

In another embodiment of the invention, the compound ABT-963 having the formula: 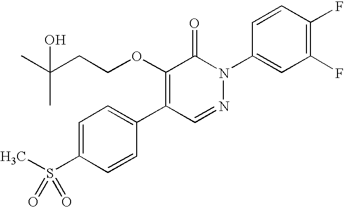
previously described in International Patent Publication No. WO 00/24719, is another tricyclic COX-2 selective inhibitor which can be advantageously employed.

Examples of specific compounds that are useful as the COX-2 selective inhibitor include, without limitation:
8-acetyl-3-(4-fluorophenyl)-2-(4-methylsulfonyl)phenyl-imidazo(1,2-a) pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl) pyrazole;
4-(5-(4-chlorophenyl)-3-(4-methoxyphenyl)-1H-pyrazol-1-yl)benzene-sulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl) benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzene-sulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzene-sulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzene-sulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzene-sulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzene-sulfonamide;
4-[5-(3-fluoro-4-methoxyphenyl)-3-(trifluoromethyl)-1H-pyrazol-1-yl]benzene-sulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzene-sulfonamide;
4-[2-(5-methylpyridin-3-yl)-4-(trifluoromethyl)-1H-imidazol-1-yl]benzene-sulfonamide;
4-[2-(2-methylpyridin-3-yl)-4-(trifluoromethyl)-1H-imidazol-1-yl]benzene-sulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzene-sulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzene-sulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzene-sulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzene-sulfonamide;
1-ally-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzene-sulfonamide;
N-phenyl-[4-(4-luorophenyl)-3-[4-(methylsulfonyl)phenyl]-5-(trifluoromethyl)-H-pyrazol-1-yl]acetamide;
ethyl[4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl) pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl)phenyl]oxazol-2-yl]-2-benzylacetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzene-sulfonamide;
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)aminolsulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
5,5-dimethyl-3-(3-fluorophenyl)-4-(4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzene-sulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4- [2-(5-methylpyridin-3-yl)-4-(trifluoromethyl)-1H-imidazol-1-yl]benzene-sulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide; 4-[5-(3-fluoro-4-methoxyphenyl-4-oxazolyl]benzenesulfonamide;
   and pharmaceutically acceptable salts and prodrugs thereof.

In a further embodiment of the invention, the Cox-2 selective inhibitor used in the present invention can be selected from the class of phenylacetic acid derivatives represented by the general structure of formula (VIII): 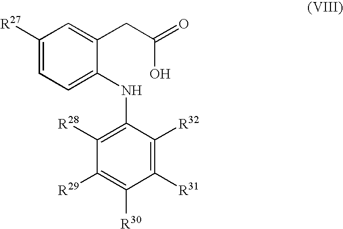
wherein:
R²⁷ is methyl, ethyl or propyl;
R²⁸ is chloro or fluoro;
R²⁹ is hydrogen, fluoro or methyl;
R³⁰ is hydrogen, fluoro, chloro, methyl, ethyl, methoxy, ethoxy or hydroxy;
R³¹ is hydrogen, fluoro or methyl; and
R³² is chloro, fluoro, trifluoromethyl, methyl, or ethyl;
   provided that R²⁸, R²⁹, R³⁰ and R³¹ are not all fluoro when R²⁷ is ethyl and R³⁰ is H; or an isomer, pharmaceutically acceptable salt, ester, or prodrug thereof.

A phenylacetic acid derivative Cox-2 selective inhibitor that is described in International Patent Publication No. WO 99/11605, incorporated by reference herein, is a compound that has the structure shown in formula (VIII), wherein R²⁷ is ethyl; R²⁸ and R³⁰ are chloro; R²⁹ and R³¹ are hydrogen; and R³² is methyl.

Another phenylacetic acid derivative Cox-2 selective inhibitor is a compound that has the structure shown in formula (VIII), wherein R²⁷ is propyl; R²⁸ and R³⁰ are chloro; R²⁹ and R³¹ are methyl; and R³² is ethyl.

Another phenylacetic acid derivative Cox-2 selective inhibitor, described in International Patent Publication No. WO 02/20090, incorporated by reference herein, is COX-189, also known as lumiracoxib, having the structure shown in formula (VIII), wherein R²⁷ is methyl; R²⁸ is fluoro; R³² is chloro; and R²⁹, R³⁰, and R³¹ are hydrogen.

Cox-2 selective inhibitor compounds that have a structure similar to that shown in formula (VIII) are described in the patents individually cited below and incorporated herein by reference.

U.S. Pat. No. 6,310,099.

U.S. Pat. No. 6,291,523.

U.S. Pat. No. 5,958,978.

Other Cox-2 selective inhibitors that can be used in the present invention have the general structure shown in formula (IX), wherein the J group is a carbocycle or a heterocycle. Illustrative embodiments have the structure: 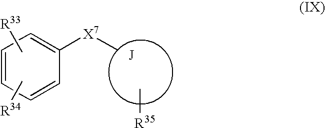
wherein:
X is O; J is 1-phenyl; R³³ is 2-NHSO₂CH₃; R³⁴ is 4-NO₂; and there is no R³⁵ group (nimesulide);
X is O; J is 1-oxo-inden-5-yl; R³³ is 2-F; R³⁴ is 4-F; and R³⁵ is 6-NHSO₂CH₃ (flosulide);
X is O; J is cyclohexyl; R³³ is 2-NHSO₂CH₃; R³⁴ is 5-NO₂; and there is no R³⁵ group (NS-398 or N-(2-cyclohexyloxynitrophenyl)methanesulfonamide);
X is S; J is 1-oxo-inden-5-yl; R³³ is 2-F; R³⁴ is 4-F; and R³⁵ is 6-N⁻SO₂CH₃.Na⁺ (L-745337);
X is S; J is thiophen-2-yl; R³³ is 4-F; there is no R³⁴ group; and R³⁵ is 5-NHSO₂CH₃ (RWJ-63556); or
X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄ (L-784512).

Materials that can serve as the Cox-2 selective inhibitor of the present invention include diarylmethylidenefuran derivatives that are described in U.S. Pat. No. 6,180,651. Such diarylmethylidenefuran derivatives have the general formula shown below in formula (X): 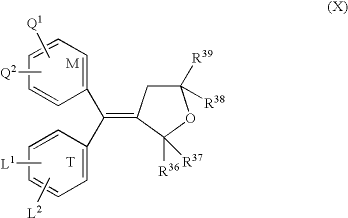
wherein:
rings T and M independently are a phenyl radical, a naphthyl radical, a radical derived from a heterocycle comprising 5 to 6 members and possessing from I to 4 heteroatoms, or a radical derived from a saturated hydrocarbon ring having from 3 to 7 carbon atoms;
at least one of the substituents Q¹, Q², L¹ and L² is (a) an —S(O)ₙ—R group, in which n is an integer equal to 0, 1 or 2 and R is a lower alkyl radical having 1 to 6 carbon atoms or a lower haloalkyl radical having 1 to 6 carbon atoms, or (b) an —SO₂NH₂ group, and is located in the para position; the others independently being a hydrogen atom, a halogen atom, a lower alkyl radical having 1 to 6 carbon atoms, a trifluoromethyl radical, or a lower O-alkyl radical having 1 to 6 carbon atoms, or Q¹ and Q² or L¹ and L² form a methylenedioxy group; and
R³⁶, R³⁷, R³⁸ and R³⁹ independently are a hydrogen atom, a halogen atom, a lower alkyl radical having 1 to 6 carbon atoms, a lower haloalkyl radical having 1 to 6 carbon atoms, or an aromatic radical selected from the group consisting of phenyl, naphthyl, thienyl, furyl and pyridyl; or R³⁶ and R³⁷, or R³⁸ and R³⁹ are an oxygen atom, or R³⁶ and R³⁷, or R³⁸ and R³⁹, together with the carbon atom to which they are attached, form a saturated hydrocarbon ring having from 3 to 7 carbon atoms;
   or an isomer or prodrug thereof.

Particular compounds of this family of compounds, which can serve as the Cox-2 selective inhibitor in the present invention, include N-(2-cyclohexyloxynitrophenyl)methanesulfonamide and (E)-4-[(4-methylphenyl) (tetrahydro-2-oxo-3-furanylidene)methyl]benzenesulfonamide.

Cox-2 selective inhibitors that are useful in the present invention include darbufelone of Pfizer, CS-502 of Sankyo, LAS 34475 and LAS 34555 of Almirall Profesfarma, S-33516 of Servier, SD-8381 of Pharmacia, described in U.S. Pat. No. 6,034,256, BMS-347070 of Bristol Myers Squibb, described in U.S. Pat. No. 6,180,651, MK-966 of Merck, L-783003 and L-748731 of Merck, T-614 of Toyama, D-1367 of Chiroscience, CT3 of Atlantic Pharmaceutical, CGP-28238 of Novartis, BF-389 of Biofor/Scherer, GR-253035 of Glaxo Wellcome, 6-dioxo-9H-purin-8-yl cinnamic acid of Glaxo Wellcome, and S-2474 of Shionogi.

Information about S-33516, mentioned above, can be found in Current Drugs Headline News, at http://www.current-drugs.com/NEWS/Inflam1.htm (2001), where it was reported that S-33516 has IC₅₀ values of 0.1 and 0.001 mM against Cox-1 and Cox-2 respectively.

Compounds that can act as Cox-2 selective inhibitors include multibinding compounds containing from 2 to 10 ligands covalently attached to one or more linkers, as described in U.S. Pat. No. 6,395,724.

Compounds that can act as Cox-2 inhibitors include a conjugated linoleic acid as described in U.S. Pat. No. 6,077,868.

Compounds that can act as Cox-2 selective inhibitors include heterocyclic aromatic oxazole compounds as described in the patents individually cited below and incorporated herein by reference.

U.S. Pat. No. 5,994,381.

U.S. Pat. No. 6,362,209.

Such heterocyclic aromatic oxazole compounds have the formula shown below in formula (XI): 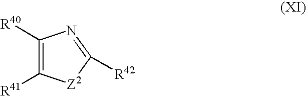
wherein:
Z² is an oxygen atom;
one of R⁴⁰ and R⁴¹ is a group of the formula 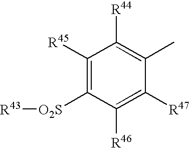
   wherein R⁴³ is lower alkyl, amino or lower alkylamino; and R⁴⁴, R⁴⁵, R⁴⁶ and R⁴⁷ are the same or different and each is hydrogen, halogen, lower alkyl, lower alkoxy, trifluoromethyl, hydroxy or amino, provided that at least one of R⁴⁴, R⁴⁵ , R⁴⁶ and R⁴⁷ is not hydrogen;
the other of R⁴⁰ and R⁴¹ is an optionally substituted cycloalkyl, heterocyclyl or aryl; and
R⁴² is a lower alkyl or a halogenated lower alkyl,
   or a pharmaceutically acceptable salt thereof.

Cox-2 selective inhibitors useful herein include compounds described in the patents individually cited below and incorporated herein by reference.

U.S. Pat. No. 6,080,876.

U.S. Pat. No. 6,133,292.

Such compounds are described by formula (XII): 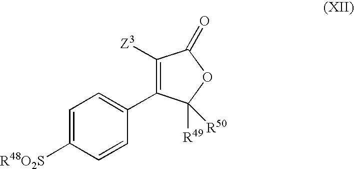
wherein:
Z³ is selected from the group consisting of (a) linear or branched C₁₋₆ alkyl, (b) linear or branched C₁₋₆ alkoxy, (c) unsubstituted, mono-, di- or tri-substituted phenyl or naphthyl wherein the substituents are selected from the group consisting of hydrogen, halo, C₁₋₃ alkoxy, CN, C₁₋₃ fluoroalkyl, C₁₋₃ alkyl and —CO₂H;
R⁴⁸ is selected from the group consisting of NH₂ and CH₃,
R⁴⁹ is selected from the group consisting of C₁₋₆ alkyl unsubstituted or substituted with C₃₋₆ cycloalkyl, and C₃₋₆ cycloalkyl;
R⁵⁰ is selected from the group consisting of C₁₋₆ alkyl unsubstituted or substituted with one, two or three fluoro atoms; and C₃₋₆ cycloalkyl;
with the proviso that R⁴⁹ and R⁵⁰ are not the same.

Compounds that can act as Cox-2 selective inhibitors include pyridines described in the patents individually cited below and incorporated herein by reference.

U.S. Pat. No. 6,369,275.

U.S. Pat. No. 6,127,545.

U.S. Pat. No. 6,130,334.

U.S. Pat. No. 6,204,387.

U.S. Pat. No. 6,071,936.

U.S. Pat. No. 6,001,843.

U.S. Pat. No. 6,040,450.

Such compounds have the general formula described by formula (XIII): 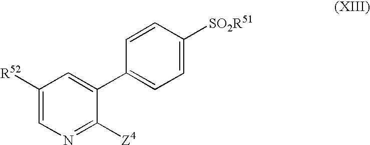
wherein:
R⁵¹ is selected from the group consisting of: CH₃, NH₂, NHC(O)CF₃ and NHCH₃;
Z⁴ is a mono-, di-, or trisubstituted phenyl or pyridinyl (or the N-oxide thereof), having substituents selected from the group consisting of hydrogen, halo, C₁₋₆ alkoxy, C₁₋₆ alkylthio, CN, C₁₋₆ alkyl, C₁₋₆ fluoroalkyl, N₃, —CO₂R⁵³, hydroxy, —C(R⁵⁴)(R⁵⁵)—OH, —C₁₋₆alkyl-CO₂—R⁵⁶ and C₁₋₆ fluoroalkoxy;
R⁵² is selected from the group consisting of halo, C₁₋₆ alkoxy, C₁₋₆ alkylthio, CN, C₁₋₆ alkyl, C₁₋₆ fluoroalkyl, N₃, —CO₂R⁵⁷, hydroxy, —C(R⁵⁸)(R⁵⁹)—OH, —C₁₋₆alkyl-CO₂—R⁶⁰, C₁₋₆ fluoroalkoxy, NO₂, NR⁶¹R⁶² and NHCOR⁶³; and
R⁵³, R⁵⁴, R⁵⁵, R⁵⁶, R⁵⁷, R⁵⁸, R⁵⁹, R⁶⁰, R⁶¹, R⁶² and R⁶³ are each independently selected from the group consisting of hydrogen and C₁₋₆ alkyl; or R⁵⁴ and R⁵⁵, R⁵⁸ and R⁵⁹, or R⁶¹ and R⁶², together with the atom to which they are attached, form a saturated monocyclic ring of 3, 4, 5, 6 or 7 atoms.

Compounds that can act as Cox-2 selective inhibitors include diarylbenzopyran derivatives as described in U.S. Pat. No. 6,340,694, incorporated herein by reference. Such diarylbenzopyran derivatives have the general formula shown below in formula (XIV): 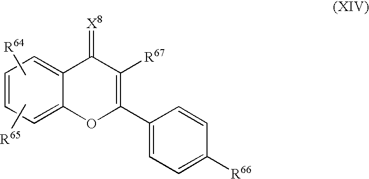
wherein:
X⁸ is an oxygen atom or a sulfur atom;
R⁶⁴ and R⁶⁵, identical to or different from each other, are independently hydrogen, halogen, C₁-C₆ lower alkyl, trifluoromethyl, alkoxy, hydroxy, nitro, nitrile or carboxyl;
R⁶⁶ is a group of a formula S(O)ₙR⁶⁸ where n is an integer of 0 to 2, R⁶⁸ is hydrogen, C₁-C₆ lower alkyl, or a group of formula NR⁶⁹R⁷⁰ wherein R⁶⁹ and R⁷⁰, identical to or different from each other, are independently hydrogen or C₁-C₆ lower alkyl group; and
R⁶⁷ is oxazolyl, benzo[b]thienyl, furanyl, thienyl, naphthyl, thiazolyl, indolyl, pyrrolyl, benzofuranyl, pyrazolyl, pyrazolyl substituted with a C₁-C₆ lower alkyl group, indanyl, pyrazinyl, or a substituted group represented by one of the following structures: 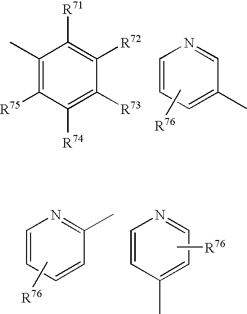
   wherein R⁷¹ through R⁷⁵, identical to or different from one another, are independently hydrogen, halogen, C₁-C₆ lower alkyl, trifluoromethyl, alkoxy, hydroxy, hydroxyalkyl, nitro, a group of formula S(O)ₙR⁶⁸, a group of formula NR⁶⁹R⁷⁰, trifluoromethoxy, nitrile, carboxyl, acetyl or formyl, wherein n, R⁶⁸, R⁶⁹ and R⁷⁰ have the same meaning as defined by R⁶⁶ above; and R⁷⁶ is hydrogen, halogen, C₁-C₆ lower alkyl, trifluoromethyl, alkoxy, hydroxy, trifluoromethoxy, carboxyl or acetyl.

Compounds that can act as Cox-2 selective inhibitors include 1-(4-sulfamylaryl)-3-substituted-5-aryl-2-pyrazolines as described in U.S. Pat. No. 6,376,519, incorporated herein by reference. Such compounds have the formula shown below in formula (XV): 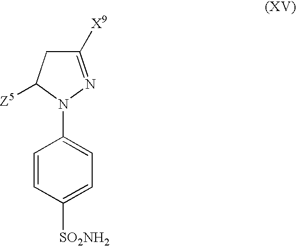
wherein:
X⁹ is selected from the group consisting of C₁-C₆ trihalomethyl, for example trifluoromethyl C₁-C₆ alkyl; and
an optionally substituted or di-substituted phenyl group of formula 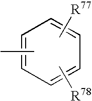
   wherein R⁷⁷ and R⁷⁸ are independently selected from the group consisting of hydrogen, halogen (e.g., chlorine, fluorine or bromine), hydroxyl, nitro, C₁-C₆ (e.g., C₁-C₃) alkyl, C₁-C₆ (e.g., C₁-C₃)alkoxy, carboxy, C₁-C₆ trihaloalkyl (e.g., trihalomethyl such as trifluoromethyl), and cyano; and
Z⁵ is selected from the group consisting of substituted and unsubstituted aryl.

Compounds that can act as Cox-2 selective inhibitors of the present invention include heterocycles as described in U.S. Pat. No. 6,153,787, incorporated herein by reference. Such heterocycles have the general formulas shown below in formulas (XVI) and (XVII): 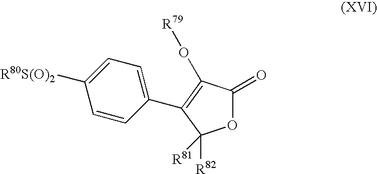
wherein:
R⁷⁹ is mono-, di- or tri-substituted C₁₋₁₂ alkyl, unsubstituted or mono-, di- or tri-substituted linear or branched C₂₋₁₀ alkenyl, unsubstituted or mono-, di- or tri-substituted linear or branched C₂₁₀ alkynyl, unsubstituted or mono-, di- or tri-substituted C₃₋₁₂ cycloalkenyl, or unsubstituted or mono-, di- or tri-substituted C₅₋₁₂ cycloalkynyl, wherein the substituents are chosen from the group consisting of halo (selected from F, Cl, Br, and I), OH, CF₃, C₃₋₆ cycloalkyl, ═O, dioxolane and CN;
R⁸⁰ is selected from the group consisting of: CH₃, NH₂, NHC(O)CF₃ and NHCH₃; and
R⁸¹ and R⁸² are independently chosen from the group consisting of hydrogen and C₁₋₁₀ alkyl; or R⁸¹ and R⁸² together with the carbon to which they are attached form a saturated monocyclic carbon ring of 3, 4, 5, 6 or 7 atoms.

Formula (XVII) is: 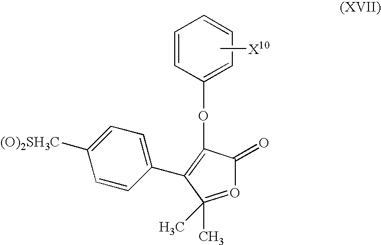
wherein X¹⁰ is fluoro or chloro.

Compounds that can act as Cox-2 selective inhibitors include 2,3,5-trisubstituted pyridines as described in U.S. Pat. No. 6,046,217, incorporated herein by reference. Such compounds have the general formula shown below in formula (XVIII): 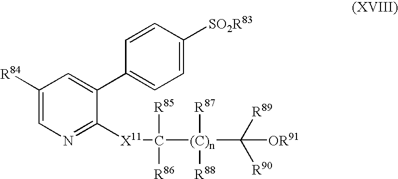
or a pharmaceutically acceptable salt thereof, wherein:
X¹¹ is selected from the group consisting of O, S and bond;
n is 0 or 1;
R⁸³ is selected from the group consisting of CH₃, NH₂ and NHC(O)CF₃;
R⁸⁴ is selected from the group consisting of halo, C₁₋₆ alkoxy, C₁₋₆ alkylthio, CN, C₁₋₆ alkyl, C₁₋₆ fluoroalkyl, N₃, —CO₂R⁹², hydroxy, —C(R⁹³)(R⁹⁴)—OH, C₁₋₆ alkyl-CO₂—R⁹⁵, C₁₋₆ fluoroalkoxy, NO₂, NR⁹⁶R⁹⁷ and NHCOR⁹⁸;
R⁸⁵ to R⁹⁸ are independently chosen from the group consisting of hydrogen and C₁₋₆ alkyl; or R⁸⁵ and R⁸⁹, or R⁸⁹ and R⁹⁰, together with the atoms to which they are attached, form a carbocyclic ring of 3, 4, 5, 6 or 7 atoms; or R⁸⁵ and R⁸⁷ are joined to form a bond.

In one exemplary embodiment of the Cox-2 selective inhibitor of formula (XVIII), X is a bond.

In another exemplary embodiment of the Cox-2 selective inhibitor of formula (XVIII), X is O.

In another exemplary embodiment of the Cox-2 selective inhibitor of formula (XVIII), X is S.

In another exemplary embodiment of the Cox-2 selective inhibitor of formula (XVIII), R⁸³ is CH₃.

In another exemplary embodiment of the Cox-2 selective inhibitor of formula (XVIII), R⁸⁴ is halo or C₁₋₆ fluoroalkyl.

Compounds that can act as Cox-2 selective inhibitors include diaryl bicyclic heterocycles as described in U.S. Pat. No. 6,329,421. Such compounds have the general formula shown below in formula (XIX): 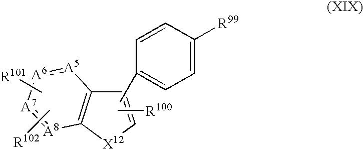
and pharmaceutically acceptable salts thereof, wherein:
-A⁵=A⁶-A⁷=A⁸- is selected from the group consisting of (a) —CH═CH—CH═CH—, (b) —CH₂—CH₂—CH₂—C(O)—, —CH₂—CH₂—C(O)—CH₂—, —CH₂—C(O)—CH₂—CH₂, —C(O)—CH₂—CH₂—CH₂, (c) —CH₂—CH₂—C(O)—, —CH₂—C(O)—CH₂—, —C(O)—CH₂—CH₂—, (d) —CH₂CH₂—O—C(O)—, —CH₂—O—C(O)—CH₂—, —O—C(O)—CH₂CH₂—, (e) —CH₂—CH₂—C(O)—O—, —CH₂—C(O)—OCH₂—, —C(O)—O—CH₂—CH₂—, (f) —C(R¹⁰⁵)₂—O—C(O)—, —C(O)—O—C(R¹⁰⁵)₂—, —O—C(O)—C(R¹⁰⁵)₂—, —C(R¹⁰⁵)₂—C(O)—O—, (g) —N═CH—CH═CH—, (h) —CH═N—CH═CH—, (i) —CH═CH—N═CH—, (j) —CH═CH—CH═N—, (k) —N═CH—CH═N—, (l) —N═CH—N═CH—, (m) —CH═N—CH═N—, (n) —S—CH═N—, (o) —S—N═CH—, (p) —N═N—NH—, (q) —CH═N—S— and (r) —N═CH—S—;
R⁹⁹ is selected from the group consisting of S(O)₂CH₃, S(O)₂NH₂, S(O)₂NHCOCF₃, S(O)(NH)CH₃, S(O)(NH)NH₂, S(O)(NH)NHCOCF₃, P(O)(CH₃)OH and P(O)(CH₃)NH₂;
R¹⁰⁰ is selected from the group consisting of (a) C₁₋₆ alkyl, (b)C₃₋₇ cycloalkyl, (c) mono- or di-substituted phenyl or naphthyl where the substituent is selected from the group consisting of hydrogen, halo including F, Cl, Br and I, C₁₋₆ alkoxy, C₁₋₆ alkylthio, CN, CF₃, C₁₋₆ alkyl, N₃, —CO₂H, —CO₂-C₁₋₄ alkyl, —C(R¹⁰³)(R¹⁰⁴)—OH, —C(R¹⁰³)(R¹⁰⁴)—O—C₁₋₄ alkyl and —C₁₋₆ alkyl-CO₂-R¹⁰⁶; (d) mono- or di-substituted heteroaryl where the heteroaryl is a monocyclic aromatic ring of 5 atoms, said ring having one hetero atom which is S, O, or N, and optionally 1, 2 or 3 additional N atoms; or a monocyclic ring of 6 atoms, said ring having one hetero atom which is N, and optionally 1, 2, 3 or 4 additional N atoms; and said substituents are selected from the group consisting of hydrogen, halo including F, Cl, Br and I, C₁₋₆ alkoxy, C₁₋₆ alkylthio, CN, CF₃, C₁₋₆ alkyl, N₃, —CO₂H, —CO₂-C₁₋₄ alkyl, —C(R¹⁰³)(R¹⁰⁴)—OH and —C(R¹⁰³)(R₁₀₄)—O—C₁₋₄ alkyl; and (e) benzoheteroaryl which includes the benzo fused analogs of (d);
R¹⁰¹ and R¹⁰² are substituents residing on any position of -A⁵=A⁶-A⁷=A⁸- and are selected independently from the group consisting of hydrogen, CF₃, CN, C₁₋₆ alkyl, Q⁴, CO₂H, C(R¹⁰³)(R¹⁰⁴)OH, —O-Q⁴, —S-Q⁴, and optionally C₁₋₃ alkyl substituted —C₁₋₅ alkyl-Q³, —O—C₁₋₅ alkyl-Q³, —S—C₁₋₅ alkyl-Q³, —C₁₋₃ alkyl-O—C₁₋₃ alkyl-Q³, —C₁₋₃ alkyl-S-C₁₋₃ alkyl-Q³, —C₁₋₅ alkyl-O-Q⁴ and —C₁₋₅ alkyl-S-Q⁴, wherein the substituent resides on the alkyl chain; where Q³ is Q⁴, CO₂H or C(R¹⁰³)(R¹⁰⁴)OH and Q⁴ is CO₂-C₁₋₄ alkyl, tetrazolyl-5-yl, or C(R¹⁰³)(R¹⁰⁴)O—C₁₋₄ alkyl;
R¹⁰³, R¹⁰⁴ and R¹⁰⁵ are each independently selected from the group consisting of hydrogen and C₁₋₆ alkyl; or R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;
R¹⁰⁶ is hydrogen or C₁₋₆ alkyl;
R¹⁰⁷ is hydrogen, C₁₋₆ alkyl or aryl; and
X⁷ is O, S, NR¹⁰⁷, CO, C(R¹⁰⁷)₂, C(R¹⁰⁷)(OH), —C(R¹⁰⁷)═C(R¹⁰⁷)—, —C(R¹⁰⁷)═N— or —N═C(R¹⁰⁷)—.

Compounds that can act as Cox-2 selective inhibitors include salts of a 5-amino- or substituted amino-1,2,3-triazole compound as described in U.S. Pat. No. 6,239,137. These salts are of a class of compounds of formula (XX): 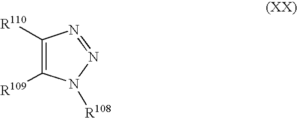
wherein:
R¹⁰⁸ is 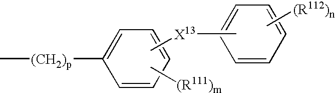
   where p is 0 to 2; m is 0 to 4; n is 0 to 5; X¹³ is O, S, SO, SO₂, CO, CHCN, CH₂, or C═NR¹¹³ where R¹¹³ is hydrogen, lower alkyl, hydroxy, lower alkoxy, amino, lower alkylamino, di(lower alkyl)amino or cyano; and R¹¹¹ and R¹¹² are independently halogen, cyano, trifluoromethyl, lower alkanoyl, nitro, lower alkyl, lower alkoxy, carboxy, lower carbalkoxy, trifluoromethoxy, acetamido, lower alkylthio, lower alkylsulfinyl, lower alkylsulfonyl, trichlorovinyl, trifluoromethylthio, trifluoromethylsulfinyl or trifluoromethyl-sulfonyl;
R¹⁰⁹ is amino, mono or di(lower alkyl)amino, acetamido, acetimido, ureido, formamido, formamido or guanidino; and
R¹¹⁰ is carbamoyl, cyano, carbazoyl, amidino or N-hydroxycarbamoyl;
wherein the lower alkyl, lower alkyl containing, lower alkoxy and lower alkanoyl groups contain from 1 to 3 carbon atoms.

Compounds that can act as Cox-2 selective inhibitors include pyrazole derivatives as described in U.S. Pat. No. 6,136,831. Such compounds have the formula shown below in formula (XXI): 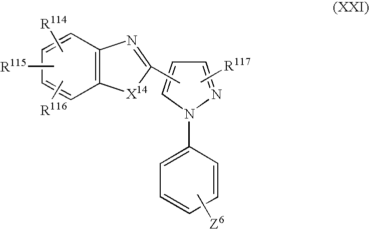
wherein:
R¹¹⁴ is hydrogen or halogen;

R¹¹⁵ and R¹¹⁶ are each independently hydrogen, halogen, lower alkyl, lower alkoxy, hydroxy or lower alkanoyloxy;
R¹¹⁷ is lower haloalkyl or lower alkyl;
X¹⁴ is sulfur, oxygen or NH; and
Z⁶ is lower alkylthio, lower alkylsulfonyl or sulfamoyl;
   or a pharmaceutically acceptable salt thereof.

Compounds that can act as Cox-2 selective inhibitors include substituted derivatives of benzosulfonamides as described in U.S. Pat. No. 6,297,282. Such compounds have the formula shown below in formula (XXII): 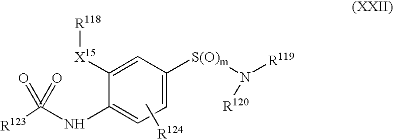
wherein:
X¹⁵ denotes oxygen, sulfur or NH;
R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;
R¹¹⁹ and R¹²⁰, independently from one another, denote hydrogen, an optionally polyfluorized alkyl group, an aralkyl, aryl or heteroaryl group or a group (CH₂)ₙ—X¹⁶; or R¹¹⁹ and R¹²⁰, together with the N atom, denote a 3- to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;
X¹⁶ denotes halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S(O)₂R¹²¹, —NR¹²¹R¹²², —NHC(O)R¹²¹ or —NHS(O)₂R¹²¹;
n denotes a whole number from 0 to 6;
R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;
R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, CO₂R¹²¹, —OCO₂R¹², —CN, —CONR¹²¹OR¹²², —CONR¹²¹R¹²², —SR¹²¹, —S(O)R¹²¹, —S(O)₂R¹²¹, —NR¹²¹R¹²², —NHC(O)R¹²¹, —NHS(O)₂R¹²¹, or a polyfluoroalkyl group;
R¹²¹ and R¹²², independently from one another, denote hydrogen, alkyl, aralkyl or aryl; and
m denotes a whole number from 0 to 2;
   and pharmaceutically-acceptable salts thereof.

Compounds that can act as Cox-2 selective inhibitors include 3-phenyl-4-(4(methylsulfonyl)phenyl)-2-(5H)-furanones as described in U.S. Pat. No. 6,239,173. Such compounds have formula (XXIII): 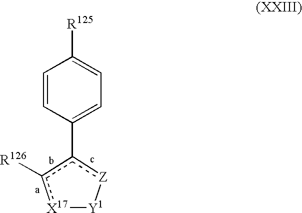
or pharmaceutically acceptable salts thereof, wherein:
—X¹⁷—Y¹-Z⁷-, when side b is a double bond, and sides a and c are single bonds, is selected from the group consisting of —CH₂CH₂CH₂—, —C(O)CH₂CH₂—, —CH₂CH₂C(O)—, —CR¹²⁹(R^{129′})—O—C(O)—, —C(O)—O—CR¹²⁹(R^{129′})—, —CH₂—NR¹²⁷—CH₂—, —CR¹²⁹(R¹²⁹)—NR¹²⁷—C(O)—, —CR¹²⁸═CR^{128′}—S—, —S—CR¹²⁸═CR^{128′}—, —S—N═CH—, CH═N—S—, —N═CR¹²⁸—O—, —O—CR¹²⁸═N—, —N═CR¹²⁸—NH—, —N═CR¹²⁸—S—, —SR═CR¹²⁸═N—, —C(O)—NR¹²⁷—CR¹²⁹(R^{129′})—, —R¹²⁷N—CH═CH— provided R¹²² is not —S(O)₂CH₃, and —CH═CH—NR¹²⁷— provided R¹²⁵ is not —S(O)₂CH₃;
—X¹¹—Y¹-Z⁷-, when sides a and c are double bonds and side b is a single bond, is selected from the group consisting of ═CH—O—CH═, ═CH—NR¹²⁷—CH═, ═N—S—CH═, ═CH—S—N═, ═N—O—CH═, ═CH—O—N═, ═N—S—N═ and ═N—O—N═;
R¹²⁵ is selected from the group consisting of S(O)₂CH₃, S(O)₂NH₂, S(O)₂NHC(O)CF₃, S(O)(NH)CH₃, S(O)(NH)NH₂, S(O)(NH)NHC(O)CF₃, P(O)(CH₃)OH and P(O)(CH₃)NH₂;
R¹²⁶ is selected from the group consisting of (a) C₁₋₆ alkyl; (b) C₃, C₄, C₅, C₆ or C₇ cycloalkyl; (c) mono-, di- or tri-substituted phenyl or naphthyl, where the substituent is selected from the group consisting of hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R¹²⁹)(R¹³⁰)—OH, —C(R¹²⁹)(R¹³⁰)—O—C₁₋₄ alkyl, and —C₁₋₆ alkyl-CO₂—R¹²⁹; (d) mono-, di- or tri-substituted heteroaryl wherein the heteroaryl is a monocyclic aromatic ring of 5 atoms, said ring having one hetero atom which is S, O or N, and optionally 1, 2 or 3 additional N atoms, or the heteroaryl is a monocyclic ring of 6 atoms, said ring having one hetero atom which is N, and optionally 1, 2, 3 or 4 additional N atoms, where the substituents are selected from the group consisting of hydrogen, halo (including fluoro, chloro, bromo and iodo), C₁₋₆ alkyl, C₁₋₆ alkoxy, C₁₋₆ alkylthio, CN, CF₃, N₃, —C(R¹²⁹)(R¹³⁰)—OH, and —C(R¹²⁹)(R¹³⁰)—O—C₁₋₄ alkyl; and (e) benzoheteroaryl including the benzo-fused analogs of (d);
R¹²⁷ is selected from the group consisting of hydrogen, CF₃, CN, C₁₋₆ alkyl, hydroxy-C₁₋₆ alkyl, —C(O)—C₁₋₆ alkyl, optionally C₁₋₃ alkyl-substituted —C₁₋₅ alkyl-Q⁵, —C₁₋₃ alkyl-O-C₁₋₃ alkyl-Q⁵, —C₁₋₃ alkyl-S—C₁₋₃ alkyl-Q⁵, —C₁₋₅ alkyl-O-Q⁵ and —C₁₋₅ alkyl-S-Q⁵ where the substituent resides on the alkyl; and -Q⁵;
R¹²⁸ and R^{128′} are each independently selected from the group consisting of hydrogen, CF₃, CN, C₁₋₆ alkyl, -Q⁵, —O-Q⁵; —S-Q⁵, and optionally C₁₋₃ alkyl-substituted —C₁₋₅ alkyl-Q⁵, —O—C₁₋₅ alkyl-Q⁵, —S—C₁₋₅ alkyl-Q⁵, —C₁₋₃ alkyl-O—C₁₋₃ alkyl-Q⁵, —C₁₋₃ alkyl-S—C₁₋₃ alkyl-Q⁵, —C₁₋₅ alkyl-O-Q⁵, —C₁₋₅ alkyl-S-Q⁵ wherein the substituent resides on the alkyl;
R¹²⁹, R^{129′}, R¹³⁰, R¹³¹ and R¹³² are each independently selected from the group consisting of hydrogen and C₁₋₆ alkyl; or R¹²⁹ and R¹³⁰, or R¹³¹ and R¹³², together with the carbon to which they are attached, form a saturated monocyclic carbon ring of 3, 4, 5, 6 or 7 atoms; and
Q⁵ is CO₂H, CO₂—C₁₋₄ alkyl, tetrazolyl-5-yl, C(R¹³¹)(R¹³²)(OH) or C(R¹³¹)(R¹³²)(O—C₁₋₄ alkyl);
provided that when —X¹⁷—Y¹-Z⁷- is —S—CR¹²⁸═CR^{128′}, then R¹²⁸ and R^{R}^{128′} are other than CF₃.

Compounds that can act as Cox-2 selective inhibitors include bicyclic carbonyl indole compounds as described in U.S. Pat. No. 6,303,628. Such compounds have the formula shown below in formula (XXIV): 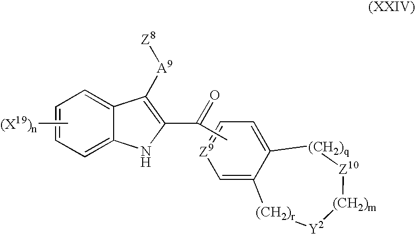
or pharmaceutically acceptable salts thereof, wherein:
A⁹ is C₁₋₆ alkylene or —NR¹³³—;
Z⁸ is C(=L³)R¹³⁴ or SO₂R¹³⁵;
Z⁹ is CH or N;
Z¹⁰ and Y² are independently selected from —CH₂—, O, S and —N—R¹³³;
m is 1, 2 or 3;
q and r are independently 0, 1 or 2;
X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di(C₁₋₄ alkyl)amino and cyano;
n is 0, 1, 2, 3 or 4;
L³ is oxygen or sulfur;
R¹³³ is hydrogen or C₁₋₄ alkyl;
R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;
R¹³⁵is C₁₋₆ alkyl or halo-substituted C₁₋₆ alkyl; and
R¹³⁶ and R¹³⁷ are independently selected from hydrogen, C₁₋₆ alkyl and halo-substituted C₁₋₆ alkyl.

Compounds that can act as a Cox-2 selective inhibitors include benzimidazole compounds as described in U.S. Pat. No. 6,310,079. Such compounds have the formula shown below in formula (XXV): 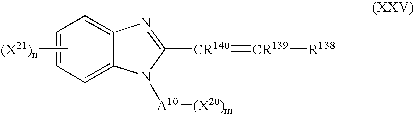
or a pharmaceutically acceptable salt thereof, wherein:
A¹⁰ is heteroaryl selected from (a) a 5-membered monocyclic aromatic ring having one hetero atom selected from O, S and N and optionally containing one to three N atom(s) in addition to said hetero atom, and (b) a 6-membered monocyclic aromatic ring having one N atom and optionally containing one to four N atom(s) in addition to said N atom; said heteroaryl being connected to the nitrogen atom on the benzimidazole through a carbon atom on the heteroaryl ring;
X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;
X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)-N-(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, N-carbamoylamino, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;
R¹³⁸ is selected from hydrogen; straight or branched C₁-C₄ alkyl optionally substituted with one to three substituent(s) independently selected from halo, hydroxy, C₁-C₄ alkoxy, amino, N-(C₁-C₄ alkyl)amino and N,N-di(C₁-C₄ alkyl)amino; C₃-C₈ cycloalkyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; C₄-C₈ cycloalkenyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; phenyl optionally substituted with one to three substituent(s) independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-[C₁-C₄ alkyl)(C₁-C₄ alkanoyl)]amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and heteroaryl selected from (a) a 5-membered monocyclic aromatic ring having one hetero atom selected from O, S and N and optionally containing one to three N atom(s) in addition to said hetero atom; and (b) a 6-membered monocyclic aromatic ring having one N atom and optionally containing one to four N) atom(s) in addition to said N atom; said heteroaryl being optionally substituted with one to three substituent(s) selected from X²⁰;
R¹³⁹ and R¹⁴⁰ are independently selected from hydrogen; halo; C₁-C₄ alkyl; phenyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; or R¹³⁸ and R¹³⁹ can form, together with the carbon atom to which they are attached, a C₃-C₇ cycloalkyl ring;
m is 0, 1, 2, 3, 4 or 5; and
n is 0, 1, 2, 3 or 4.

Compounds that can act as Cox-2 selective inhibitors include indole compounds that are described in U.S. Pat. No. 6,300,363. Such compounds have the formula shown below in formula (XXVI): 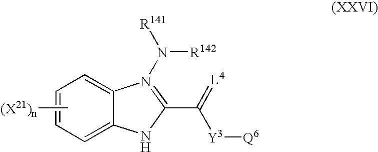
and pharmaceutically acceptable salts thereof, wherein:
L⁴ is oxygen or sulfur;
Y³ is a direct bond or C₁₋₄ alkylidene;
Q⁶ is (a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino; (b) C₃₋₇ cycloalkyl optionally substituted with up to three substituents independently selected from hydroxy, C₁₋₄ alkyl and C₁₋₄ alkoxy; (c) phenyl or naphthyl, said phenyl or naphthyl being optionally substituted with up to four substituents independently selected from halo, C₁₋₄ alkyl, halo-substituted C₁₋₄ alkyl, hydroxy, C₁₋₄ alkoxy, halo-substituted C₁₋₄ alkoxy, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN; (d) a monocyclic aromatic group of 5 atoms, said aromatic group having one heteroatom selected from O, S and N and optionally containing up to three N atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; or (e) a monocyclic aromatic group of 6 atoms, said aromatic group having one heteroatom which is N and optionally containing up to three atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³;
R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂;
R¹⁴² is hydrogen; C₁₋₄ alkyl; C(O)R¹⁴⁵ where R¹⁴⁵ is selected from (a) C₁₋₂₂ alkyl or C₂₋₂₂ alkenyl, said alkyl or alkenyl being optionally substituted with up to four substituents independently selected from halo, hydroxy, OR¹⁴³, S(O)ₘR¹⁴³, nitro, amino, mono- or di-(C₁₋₄ alkyl)amino, NHSO₂R¹⁴³, CO₂H, CO₂(C₁₋₄ alkyl), CONH₂, CONH(C₁₋₄ alkyl), CON(C₁₋₄ alkyl)₂, OC(O)R¹⁴³, thienyl, naphthyl and groups of the following formulae: 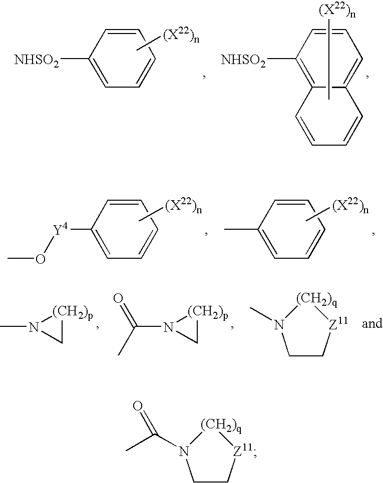
   (b) C₁₋₂₂ alkyl or C₂₋₂₂ alkenyl, said alkyl or alkenyl being optionally substituted with 5 to 45 halogen atoms; (c) —Y⁵—C₃₋₇ cycloalkyl or —Y⁵—C₃₋₇ cycloalkenyl, said cycloalkyl or cycloalkenyl being optionally substituted with up to three substituent independently selected from C₁₋₄ alkyl, hydroxy, OR¹⁴³, S(O)ₘR¹⁴³, amino, mono- or di-(C₁₋₄ alkyl)amino, CONH₂, CONH(C₁₋₄ alkyl) and CON(C₁₋₄ alkyl)₂; (d) phenyl or naphthyl, said phenyl or naphthyl being optionally substituted with up to seven substituents independently selected from halo, C₁₋₈ alkyl, C₁₋₄ alkyl-OH, hydroxy, C₁₋₈ alkoxy, halo-substituted C₁₋₈ alkyl, halo-substituted C₁₋₈ alkoxy, CN, nitro, S(O)ₘR¹⁴³, SO₂NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂(C₁₋₄ alkyl) and CONH₂; (e) a monocyclic aromatic group of 5 atoms, said aromatic group having one heteroatom selected from O, S and N and optionally containing up to three N atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; (f) a monocyclic aromatic group of 6 atoms, said aromatic group having one heteroatom which is N and optionally containing up to three atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; or (g) a group of the following formula: 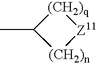
X²² is halo, C₁₋₄ alkyl, hydroxy, C₁₋₄ alkoxy, halo-substituted C₁₋₄ alkoxy, S(O)ₘR¹⁴³, amino, mono- or di-(C₁₋₄ alkyl)amino, NHSO₂ R¹⁴³, nitro, halo-substituted C₁₋₄ alkyl, CN, CO₂ H, CO₂ (C₁₋₄ alkyl), C₁₋₄ alkyl-OH, C₁₋₄ alkyl-OR¹⁴³, CONH₂, CONH(C₁₋₄ alkyl) or CON(C₁₋₄ alkyl)₂;
R¹⁴³ is C₁₋₄ alkyl or halo-substituted C₁₋₄ alkyl;
m is 0, 1 or 2;
n is 0, 1, 2 or 3;
p is 1, 2, 3, 4 or 5;
q is 2 or 3;
Z¹¹ is oxygen, sulfur or NR¹⁴⁴; and
R¹⁴⁴ is hydrogen, C₁₋₆ alkyl, halo-substituted C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘR¹⁴³, amino, mono- or di-(C₁₋₄ alkyl)amino, CF₃, OCF₃, CN and nitro;
with the proviso that a group of formula —Y⁵-Q is not methyl or ethyl when X²² is hydrogen, L⁴ is oxygen, R¹⁴¹ is hydrogen and R¹⁴² is acetyl.

Compounds that can act as Cox-2 selective inhibitors include aryl phenylhydrazides as described in U.S. Pat. No. 6,077,869. Such compounds have the formula shown below in formula (XXVII): 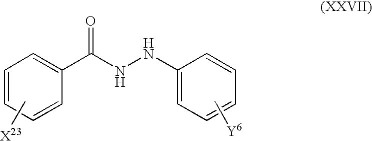
wherein X²³ and Y⁶ are selected from hydrogen, halogen, alkyl, nitro, amino and other oxygen- and sulfur-containing functional groups such as hydroxy, methoxy and methylsulfonyl.

Compounds that can act as Cox-2 selective inhibitors include 2-aryloxy-4-aryl furan-2-ones as described in U.S. Pat. No. 6,140,515. Such compounds have the formula shown below in formula (XXVIII): 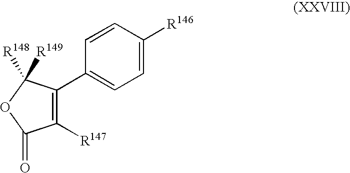
or a pharmaceutically acceptable salt thereof, wherein:
R¹⁴⁶ is selected from the group consisting of SCH₃, —S(O)₂CH₃ and —S(O)₂NH₂;
R¹⁴⁷ is selected from the group consisting of OR¹⁵⁰, mono- or di-substituted phenyl or pyridyl wherein the substituents are selected from the group consisting of methyl, chloro and fluoro;
R¹⁵⁰ is unsubstituted or mono- or di-substituted phenyl or pyridyl wherein the substituents are selected from the group consisting of methyl, chloro and fluoro;
R¹⁴⁸ is H or C₁₋₄ alkyl optionally substituted with 1 to 3 groups of F, Cl or Br; and
R¹⁴⁹ is H and C₁₋₄ alkyl optionally substituted with 1 to 3 groups of F, Cl or Br; with the proviso that R¹⁴⁸ and R¹⁴⁹ are not the same.

Compounds that can act as Cox-2 selective inhibitors include bisaryl compounds as described in U.S. Pat. No. 5,994,379. Such compounds have the formula shown below in formula (XXIX): 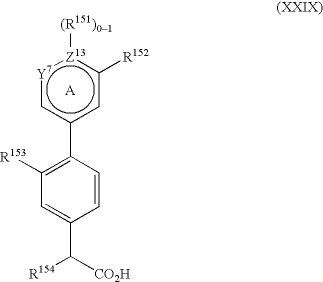
or a pharmaceutically acceptable salt, ester or tautomer thereof, wherein:
Z¹³ is C or N;
when Z¹³ is N, R¹⁵¹ represents H or is absent, or is taken in conjunction with R¹⁵² as described below;
when Z¹³ is C, R¹⁵¹ represents H and R¹⁵² is a moiety which has the following characteristics: (a) it is a linear chain of 3-4 atoms containing 0-2 double bonds, which can adopt an energetically stable transoid configuration and if a double bond is present, the bond is in the trans configuration, (b) it is lipophilic except for the atom bonded directly to ring A, which is either lipophilic or non-lipophilic, and (c) there exists an energetically stable configuration planar with ring A to within about 15 degrees; or R¹⁵¹ and R¹⁵² are taken in combination and represent a 5- or 6-membered aromatic or non-aromatic ring D fused to ring A, said ring D containing 0-3 heteroatoms selected from 0, S and N; said ring D being lipophilic except for the atoms attached directly to ring A, which are lipophilic or non-lipophilic, and said ring D having available an energetically stable configuration planar with ring A to within about 15 degrees; said ring D further being substituted with one R^{a} group selected from the group consisting of C₁₋₂ alkyl, —O—C₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;
Y⁷ represents N, CH or C—O—C₁₋₃ alkyl, and when Z¹³ is N, Y⁷ can also represent a carbonyl group;
R¹⁵³ represents H, Br, Cl or F; and
R¹⁵⁴ represents H or CH₃.

Compounds that can act as Cox-2 selective inhibitors include 1,5-diarylpyrazoles as described in U.S. Pat. No. 6,028,202. Such compounds have the formula shown below in formula (XXX): 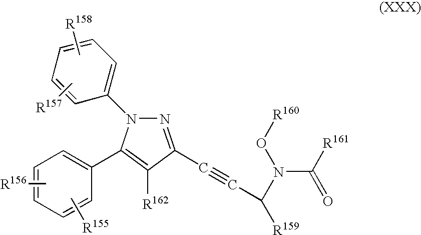
wherein:
R¹⁵⁵, R¹⁵⁶, R¹⁵⁷ and R¹⁵⁸ are independently selected from the group consisting of hydrogen, C₁₋₅ alkyl, C₁₋₅ alkoxy, phenyl, halo, hydroxy, C₁₋₅ alkylsulfonyl, C₁₋₅ alkylthio, trihalo-C₁₋₅ alkyl, amino, nitro and 2-quinolinylmethoxy;
R¹⁵⁹ is hydrogen; C₁₋₅ alkyl; trihalo-C₁₋₅ alkyl; phenyl; substituted phenyl where the phenyl substituents are halogen, C₁₋₅ alkoxy, trihalo-C₁₋₅ alkyl or nitro; or heteroaryl of 5-7 ring members where at least one of the ring members is nitrogen, sulfur or oxygen;
R¹⁶⁰ is hydrogen; C₁₋₅ alkyl; phenyl-C₁₋₅ alkyl; substituted phenyl-C₁₋₅ alkyl where the phenyl substituents are halogen, C₁₋₅ alkoxy, trihalo-C₁₋₅ alkyl or nitro; C₁₋₅ alkoxycarbonyl; phenoxycarbonyl; or substituted phenoxycarbonyl where the phenyl substituents are halogen, C₁₋₅ alkoxy, trihalo-C₁₋₅ alkyl or nitro;
R¹⁶¹ is C₁₋₁₀ alkyl; substituted Cl₁₀ alkyl where the substituents are halogen, trihalo-C₁₋₅ alkyl, C₁₋₅ alkoxy, carboxy, C₁₋₅ alkoxycarbonyl, amino, C₁₋₅ alkylamino, di(C₁₋₅ alkyl)amino, di(C₁₋₅ alkyl)amino-C₁₋₅ alkylamino, C₁₋₅ alkylamino-C₁₋₅ alkylamino or a heterocycle containing 4-8 ring atoms where one more of the ring atoms is nitrogen, oxygen or sulfur, said heterocycle being optionally substituted with C₁₋₅ alkyl; phenyl; substituted phenyl where the phenyl substituents are one or more of C₁₋₅ alkyl, halogen, C₁₋₅ alkoxy, trihalo-C₁₋₅ alkyl or nitro; heteroaryl having 5-7 ring atoms where one or more atoms are nitrogen, oxygen or sulfur; fused heteroaryl where one or more 5-7 membered aromatic rings are fused to the heteroaryl; or NR¹⁶³R¹⁶⁴ where R¹⁶³ and R¹⁶⁴ are independently selected from hydrogen and C₁₋₅ alkyl, or R¹⁶³ and R¹⁶⁴ may be taken together with the depicted nitrogen to form a heteroaryl ring of 5-7 ring members where one or more of the ring members is nitrogen, sulfur or oxygen, said heteroaryl ring being optionally substituted with C₁₋₅ alkyl; and
R¹⁶² is hydrogen, C₁₋₅ alkyl, nitro, amino or halogen;
   or pharmaceutically acceptable salts thereof.

Compounds that can act as Cox-2 selective inhibitors include 2-substituted imidazoles as described in U.S. Pat. No. 6,040,320. Such compounds have the formula shown below in formula (XXXI): 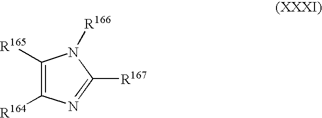
wherein:
R¹⁶⁴ is phenyl; heteroaryl containing 5 to 6 ring atoms; or substituted phenyl wherein the substituents are independently selected from one or members of the group consisting of C₁₋₅ alkyl, halogen, nitro, trifluoromethyl and nitrile;
R¹⁶⁵ is phenyl; heteroaryl containing 5 to 6 ring atoms; substituted heteroaryl wherein the substituents are independently selected from one or more members of the group consisting of C₁₋₅ alkyl and halogen; or substituted phenyl wherein the substituents are independently selected from one or members of the group consisting of C₁₋₅ alkyl, halogen, nitro, trifluoromethyl and nitrile;
R¹⁶⁶ is hydrogen, 2-(trimethylsilyl)ethoxymethyl, C₁₋₅ alkoxycarbonyl, aryloxycarbonyl, aryl-C₁₋₅ alkyloxycarbonyl, aryl-C₁₋₅ alkyl, phthalimido-C₁₋₅ alkyl, amino-C₁₋₅ alkyl, diamino-C₁₋₅ alkyl, succinimido-C₁₋₅ alkyl, C₁₋₅ alkylcarbonyl, arylcarbonyl, C₁₋₅ alkylcarbonyl-C1-5 alkyl, aryloxycarbonyl-C₁₋₅ alkyl, heteroaryl-C₁₋₅ alkyl where the heteroaryl contains 5 to 6 ring atoms, or substituted aryl-C₁₋₅ alkyl wherein the aryl substituents are independently selected from one or more members of the group consisting of C₁₋₅ alkyl, C₁₋₅ alkoxy, halogen, amino, C₁₋₅ alkylamino and di(C₁₋₅ alkyl)amino; and
R¹⁶⁷ is (A¹¹)ₙ-(CH¹⁶⁵)_{q}X²⁴ wherein A¹¹ is sulfur or carbonyl; n is 0 or 1; q is 0-9; and X²⁴ is selected from the group consisting of hydrogen; hydroxy; halogen; vinyl; ethynyl; C₁₋₅ alkyl; C₃₋₇ cycloalkyl; C₁₋₅ alkoxy; phenoxy; phenyl; aryl-C₁₋₅ alkyl; amino; C₁₋₅ alkylamino; nitrile; phthalimido; amido; phenylcarbonyl; C₁₋₅ alkylaminocarbonyl; phenylaminocarbonyl; aryl-C₁₋₅ alkylaminocarbonyl; C₁₋₅ alkylthio; C₁₋₅ alkylsulfonyl; phenylsulfonyl; substituted sulfonamido wherein the sulfonyl substituent is selected from the group consisting of C₁₋₅ alkyl, phenyl, araC₁₋₅ alkyl, thienyl, furanyl and naphthyl; substituted vinyl wherein the substituents are independently selected from one or members of the group consisting of fluorine, bromine, chlorine and iodine; substituted ethynyl wherein the substituents are independently selected from one or more members of the group consisting of fluorine, bromine, chlorine and iodine; substituted C₁₋₅ alkyl wherein the substituents are selected from the group consisting of one or more C₁₋₅ alkoxy, trihaloalkyl, phthalimido and amino; substituted phenyl wherein the phenyl substituents are independently selected from one or more members of the group consisting of C₁₋₅ alkyl, halogen and C₁₋₅ alkoxy; substituted phenoxy wherein the phenyl substituents are independently selected from one or more members of the group consisting of C₁₋₅ alkyl, halogen and C₁₋₅ alkoxy; substituted C₁₋₅ alkoxy wherein the alkyl substituent is selected from the group consisting of phthalimido and amino; substituted aryl-C₁₋₅ alkyl wherein the alkyl substituent is hydroxyl; substituted aryl-C₁₋₅ alkyl wherein the phenyl substituents are independently selected from one or more members of the group consisting of C₁₋₅ alkyl, halogen and C₁₋₅ alkoxy; substituted amido wherein the carbonyl substituent is selected from the group consisting of C₁₋₅ alkyl, phenyl, arylC₁₋₅ alkyl, thienyl, furanyl and naphthyl; substituted phenylcarbonyl wherein the phenyl substituents are independently selected from one or members of the group consisting of C₁₋₅ alkyl, halogen and C₁₋₅ alkoxy; substituted C₁₋₅ alkylthio wherein the alkyl substituent is selected from the group consisting of hydroxy and phthalimido; substituted C₁₋₅ alkylsulfonyl wherein the alkyl substituent is selected from the group consisting of hydroxy and phthalimido; and substituted phenylsulfonyl wherein the phenyl substituents are independently selected from one or members of the group consisting of bromine, fluorine, chlorine, C₁₋₅ alkoxy and trifluoromethyl; with the proviso that (a) if A¹¹ is sulfur and X²⁴ is other than hydrogen, C₁₋₅ alkylaminocarbonyl, phenylaminocarbonyl, aryl-C₁₋₅ alkylaminocarbonyl, C₁₋₅ alkylsulfonyl or phenylsulfonyl, then q must be equal to or greater than 1; (b) if A¹¹ is sulfur and q is 1, then X²⁴ cannot be C₁₋₂ alkyl; (c) if A¹¹ is carbonyl and q is 0, then X²⁴ cannot be vinyl, ethynyl, C₁₋₅ alkylaminocarbonyl, phenylaminocarbonyl, aryl-C₁₋₅ alkylaminocarbonyl, C₁₋₅ alkylsulfonyl or phenylsulfonyl; (d) if A¹¹ is carbonyl, q is 0 and X²⁴ is H, then R¹⁶⁶ is not 2-(trimethylsilyl)ethoxymethyl; (e) if n is 0 and q is 0, then X²⁴ cannot be hydrogen;
   and pharmaceutically acceptable salts thereof.

Compounds that can act as Cox-2 selective inhibitors include 1,3- and 2,3-diarylcycloalkano- and cycloalkenopyrazoles as described in U.S. Pat. No. 6,083,969. Such compounds have the general formulas (XXXII) and (XXXIII) shown below: 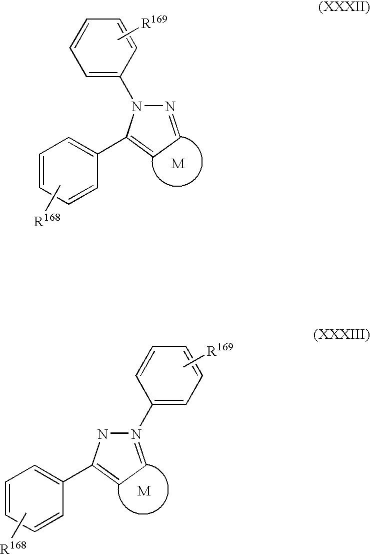
wherein:
R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and
the fused moiety M is selected from the group consisting of an optionally substituted cyclohexyl and cycloheptyl group having the formulae: 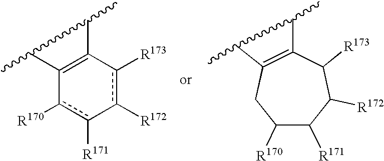
   wherein:
R¹⁷⁰ is selected from the group consisting of hydrogen, halogen, hydroxy and carbonyl;
R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, OCH₂CH₃, —OSO₂NHCO₂CH₃, ═CHCO₂CH₂CH₃, —CH₂CO₂H, —CH₂CO₂CH₃, —CH₂CO₂CH₂CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy; and
R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from halogen, hydroxy, amino, (C₁-C₆)alkyl and (C₁-C₆)alkoxy;
or R¹⁷⁰ and R¹⁷¹ taken together form a moiety selected from the group consisting of —OCOCH₂—, —ONH(CH₃)COCH₂—, —OCOCH.dbd. and —O—;
and/or R¹⁷² and R¹⁷³ taken together form a moiety selected from the group consisting of —O— and 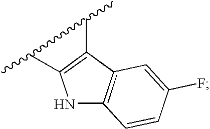
R¹⁷⁴ is selected from the group consisting of hydrogen, OH, —OCOCH₃, —COCH₃ and (C₁-C₆)alkyl; and
R¹⁷⁵ is selected from the group consisting of hydrogen, OH, —OCOCH₃, —COCH₃, (C₁-C₆)alkyl, —CONH₂ and —SO₂CH₃;
with the proviso that if M is a cyclohexyl group, then R¹⁷⁰ through R¹⁷³ may not all be hydrogen; and pharmaceutically acceptable salts, esters and pro-drug forms thereof.

Compounds that can serve as Cox-2 selective inhibitors include esters derived from indolealkanols and amides derived from indolealkylamides as described in U.S. Pat. No. 6,306,890. Such compounds have the general formula shown below in formula (XXXIV): 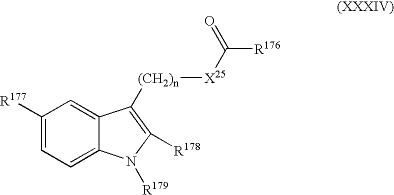
wherein:
R¹⁷⁶ is C₁-C₆ alkyl, C₁-C₆ branched alkyl, C₄-C₈ cycloalkyl, C₁-C₆ hydroxyalkyl, branched C₁-C₆ hydroxyalkyl, hydroxy-substituted C₄-C₈ aryl, primary, secondary or tertiary C₁-C₆ alkylamino, primary, secondary or tertiary branched C₁-C₆ alkylamino, primary, secondary or tertiary C₄-C₈ arylamino, C₁-C₆ alkylcarboxylic acid, branched C₁-C₆ alkylcarboxylic acid, C₁-C₆ alkylester, branched C₁-C₆ alkylester, C₄-C₈ aryl, C₄-C₈ arylcarboxylic acid, C₄-C₈ arylester, C₄-C₈ aryl-substituted C₁-C₆ alkyl, C₄-C₈ heterocyclic alkyl or aryl with O, N or S in the ring, alkyl-substituted or aryl-substituted C₄-C₈ heterocyclic alkyl or aryl with O, N or S in the ring, or halo-substituted versions thereof, where halo is chloro, bromo, fluoro or iodo;
R¹⁷⁷ is halo, C₁-C₆ alkyl, C₁-C₆ branched alkyl, C₄-C₈ cycloalkyl, C₄-C₈ aryl, C₄-C₈ aryl-substituted C₁-C₆ alkyl, C₁-C₆ alkoxy, C₁-C₆ branched alkoxy, C₄-C₈ aryloxy, or halo-substituted versions thereof, where halo is chloro, fluoro, bromo, or iodo;
R¹⁷⁸ is hydrogen, C₁-C₆ alkyl or C₁-C₆ branched alkyl;
R¹⁷⁹ is C₁-C₆ alkyl, C₄-C₈ aroyl, C₄-C₈ aryl, C₄-C₈ heterocyclic alkyl or aryl with aryl-substituted C₁-C₆ alkyl, alkyl-substituted or aryl-substituted C₄-C₈ heterocyclic alkyl or aryl with O, N or S in the ring, alkyl-substituted C₄-C₈ aroyl, or alkyl-substituted C₄-C₈ aryl, or halo-substituted versions thereof where halo is chloro, bromo, or iodo;
n is 1, 2, 3, or 4; and
X²⁵ is O, NH, or N—R¹⁸⁰, where R¹⁸⁰ is C₁-C₆ alkyl or C₁-C₆ branched alkyl.

Compounds that can act as Cox-2 selective inhibitors include pyridazinone compounds as described in U.S. Pat. No. 6,307,047. Such compounds have the formula (XXXV): 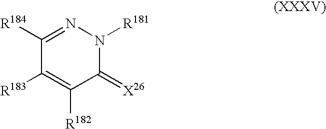
or a pharmaceutically acceptable salt, ester, or prodrug thereof, wherein:
X²⁶ is selected from the group consisting of O, S, —NR¹⁸¹, —NOR^{a} and —NNR^{b}R^{c};
R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;
R^{a}, R^{b} and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —CH₂)ₙCH(OH)R¹⁸⁶, —(CH₂)ₙC(NOR^{d})R¹⁸⁶, —(CH₂)ₙCH(NOR^{d})R¹⁸⁶, —(CH₂)ₙCH(NR^{d}R^{e})R¹⁸⁶, —R¹⁸⁷R¹⁸⁸, —(CH₂)ₙC≡CR¹⁸⁸, —(CH₂)ₙ[CH(CX^{26′}₃)]ₘ(CH₂)ₚR¹⁸⁸, —(CH₂)ₙ(CX^{26′})ₘ(CH₂)ₘR¹⁸⁸;
R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;
R¹⁸⁷ is selected from the group consisting of alkenylene, alkylene, halo-substituted alkenylene, and halo-substituted alkylene;
R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;
R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;
X^{26′} is halogen;
m is an integer from 0 to 5;
n is an integer from 0 to 10;
p is an integer from 0 to 10;
R¹⁸², R¹⁸³ and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸ and Z¹⁴, provided that one of R¹⁸², R¹⁸³ or R¹⁸⁴ must be Z¹⁴, and further provided that only one of R¹⁸², R¹⁸³ or R¹⁸⁴ is Z¹⁴;
Z¹⁴ is selected from the group consisting of: 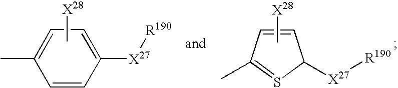
X²⁷ is selected from the group consisting of S(O)₂, S(O)(NR¹⁹¹), S(O), Se(O)₂, P(O)(OR¹⁹²) and P(O)(NR¹⁹³R¹⁹⁴);
X²⁸ is selected from the group consisting of hydrogen, alkenyl, alkyl, alkynyl and halogen;
R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂ and —NCHN(R¹⁹¹)R¹⁹²;
R¹⁹¹, R¹⁹², R¹⁹³ and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing I or 2 heteroatoms selected from the group consisting of O, S, and NR¹⁸⁸;
Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵ and —N(R¹⁹⁷)R¹⁹⁵;
R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl and NR¹⁹⁹R²⁰⁰; and
R¹⁹⁷, R¹⁹⁸, R¹⁹⁹ and R²⁰⁰ are independently selected from the group consisting of hydrogen, alkenyl, alkoxy, alkyl, cycloalkenyl, cycloalkyl, aryl, arylalkyl, heterocyclic and heterocyclic alkyl.

Compounds that can act as Cox-2 selective inhibitors include benzosulfonamide derivatives as described in U.S. Pat. No. 6,004,948. Such compounds have the formula (XXXVI): 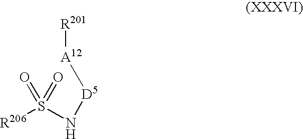
wherein:
A¹² denotes oxygen, sulfur or NH;
R²⁰¹ denotes a cycloalkyl, aryl or heteroaryl group optionally mono- or polysubstituted by halogen, alkyl, CF₃ or alkoxy;
D⁵ denotes a group: 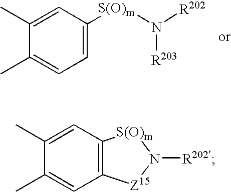
R²⁰² and R²⁰³ independently of each other denote hydrogen, an optionally polyfluorinated alkyl radical, an aralkyl, aryl or heteroaryl radical or a radical (CH₂)ₙ—X²⁹; or R²⁰² and R²⁰³ together with the N-atom denote a 3- to 7-membered, saturated, partially or totally unsaturated heterocycle with one or more heteroatoms N, O, or S, which can optionally be substituted by oxo, an alkyl, alkylaryl or aryl group or a group (CH₂)ₙ—X²⁹;
R²⁰², denotes hydrogen, an optionally polyfluorinated alkyl group, an aralkyl, aryl or heteroaryl group or a group (CH₂)ₙ—X²⁹;
X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵, —CONR²⁰⁴R²⁰⁵, —SR²⁰⁴, —S(O)R²⁰⁴, —S(O)₂R²⁰⁴, —NR²⁰⁴R²⁰⁵, —NHC(O)²⁰⁴ or —NHS(O)₂R²⁰⁴;
Z¹⁵ denotes —CH₂—, —CH₂CH₂—, —CH₂—CH₂—CH₂—, —CH₂—CH═CH—, —CH═CH—CH₂—, —CH₂—CO—, —CO—CH₂, —NHCO—, —CONH—, —NHCH₂, —CH₂NH—, —N═CH—, —NHCH—, —CH₂—CH₂—NH—, —CH═CH—, >N—R²⁰³, >C═O or >S(O)ₘ;
R²⁰⁴ and R²⁰⁵ independently of each other denote hydrogen, alkyl, aralkyl or aryl;
n is an integer from 0 to 6;
R²⁰⁶ is CF₃ or a straight-chained or branched C₁₋₄ alkyl group optionally mono- or polysubstituted by halogen or alkoxy; and
m denotes an integer from 0 to 2;
with the proviso that A¹² does not represent O if R²⁰⁶ denotes CF₃;
   and pharmaceutically acceptable salts thereof.

Compounds that can act as Cox-2 selective inhibitors include methanesulfonyl-biphenyl derivatives as described in U.S. Pat. No. 6,583,321. Such compounds have the formula (XXXVII): 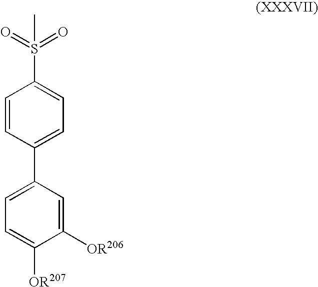
wherein R²⁰⁷ and R²⁰⁸ are individually hydrogen; C₁-C₄ alkyl, substituted or not substituted by halogen atoms; C₃-C₇ cycloalkyl; C₁-C₅ alkyl containing 1-3 ether bonds and/or an aryl substitute; substituted or unsubstituted phenyl; or substituted or unsubstituted 5- or 6-ring-cycled heteroaryl containing more than one hetero atom selected from the group consisting of nitrogen, sulfur and oxygen (wherein phenyl or heteroaryl can be mono- or multi-substituted by a substituent selected from the group consisting of hydrogen, methyl, ethyl and isopropyl).

Compounds that can act as Cox-2 selective inhibitors include 1H-indole derivatives as described in U.S. Pat. No. 6,599,929. Such compounds have the formula (XXXVIII): 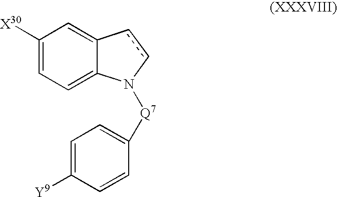
wherein:
X³⁰ is —NHSO₂R²⁰¹ wherein R²⁰⁹ represents hydrogen or C₁-C₃ alkyl;
Y⁹ is hydrogen, halogen, C₁-C₃ alkyl substituted or not substituted by halogen atoms, NO₂, NH₂, OH, OMe, CO₂H or CN; and
Q⁷ is C═O, C═S or CH₂.

Compounds that can act as Cox-2 selective inhibitors include prodrugs as described in U.S. Pat. No. 6,436,967 and U.S. Pat. No. 6,613,790. Such compounds have the formula (XXXIX): 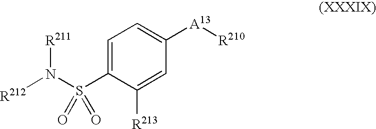
wherein:
A¹³ is a ring substituent selected from partially unsaturated heterocyclic, heteroaryl, cycloalkenyl and aryl, wherein A¹³ is unsubstituted or substituted with one or more radicals selected from alkylcarbonyl, formyl, halo, alkyl, haloalkyl, oxo, cyano, nitro, carboxyl, alkoxy, aminocarbonyl, alkoxycarbonyl, carboxyalkyl, cyanoalkyl, hydroxyalkyl, haloalkylsulfonyloxy, alkoxyalkyloxyalkyl, carboxyalkoxyalkyl, cycloalkylalkyl, alkenyl, alkynyl, heterocycloxy, alkylthio, cycloalkyl, aryl, heterocyclyl, cycloalkenyl, aralkyl, heterocyclylalkyl, alkylthioalkyl, arylcarbonyl, aralkylcarbonyl, aralkenyl, alkoxyalkyl, arylthioalkyl, aryloxyalkyl, aralkylthioalkyl, araalkoxyalkyl, alkoxycarbonylalkyl, aminocarbonylalkyl, alkylaminocarbonyl, N-arylaminocarbonyl, N-alkyl-N-arylaminocarbonyl, alkylaminocarbonylalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkyl-aminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl;
R²¹⁰ is selected from heterocyclyl, cycloalkyl, cycloalkenyl and aryl, wherein R²¹⁰ is unsubstituted or substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²¹¹ is selected from hydrido and alkoxycarbonylalkyl;
R²¹² is selected from alkyl, carboxyalkyl, acyl, alkoxycarbonyl, heteroarylcarbonyl, alkoxycarbonylalkylcarbonyl, alkoxycarbonylcarbonyl, amino acid residue and alkylcarbonylamino-alkylcarbonyl;
provided A¹³ is not tetrazolium or pyridinium; further provided A¹³ is not indanone when R²¹² is alkyl or carboxyalkyl; and further provided A¹³ is not thienyl when R²¹⁰ is 4-fluorophenyl, R²¹¹ is hydrido and R²¹² is methyl or acyl; and
R²¹³ is hydrido;
   and pharmaceutically acceptable salts thereof.

Specific non-limiting examples of substituted sulfonamide prodrugs of Cox-2 inhibitors disclosed in U.S. Pat. No. 6,436,967 that are useful in the present invention include:
N-[[4-[3-(difluoromethyl)-5-(3-fluoro-4-methoxyphenyl)-1H-pyrazol-1-yl]phenyl]sulfonyl]propanamide;
N-[[4-[3-(difluoromethyl)-5-(3-fluoro-4-methoxyphenyl)-1H-pyrazol-1-yl]phenyl]sulfonyl]butanamide;
N-[[4-[1,5-dimethyl)-3-phenyl-1H-pyrazol-4-yl]phenyl]sulfonyl]acetamide;
N-[[4-(2-(3-pyridinyl)-4-(trifluoromethyl)-1H-imidazol-1-yl)phenyl]sulfonyl]acetamide;
N-[[4-[2-(5-methylpyridin-3-yl)-4-(trifluoromethyl)-1H-imidazol-1-yl]phenyl]sulfonyl]acetamide;
N-[[4-[2-(2-methylpyridin-3-yl)-4-(trifluoromethyl)-1H-imidazol-1-yl]phenyl]sulfonyl]acetamide;
N-[[4-[2-(5-methylpyridin-3-yl)-4-(trifluoromethyl)-1H-imidazol-1-yl]phenyl]sulfonyl]butanamide;
N-[[4-[2-(2-methylpyridin-3-yl)-4-(trifluoromethyl)-1H-imidazol-1-yl]phenyl]sulfonyl]butanamide;
N-[[4-[2-(3-chloro-5-methylphenyl)-4-(trifluoromethyl)-1H-imidazol-1-yl]phenyl]sulfonyl]acetamide;
N-[[4-[3-(3-fluorophenyl)-5-methylisoxazol-4-yl]phenyl]sulfonyl]acetamide;
2-methyl-N-[[4-(5-methyl-3-phenylisoxazol-4-yl)phenyl]sulfonyl]propanamide;
N-[[4-(5-methyl-3-phenylisoxazol-4-yl]phenyl]sulfonyl]propanamide;
N-[[4-(5-methyl-3-phenylisoxazol-4-yl)phenyl]sulfonyl]benzamide;
2,2-dimethyl-N-[[4-(5-methyl-3-phenylisoxazol-4-yl)phenyl]sulfonyl]propanamide;
N-[[4-5-methyl-3-phenylisoxazol-4-yl)phenyl]sulfonyl]butanamide;
N-[[4-(5-methyl-3-phenylisoxazol-4-yl)phenyl]sulfonyl]pentanamide;
N-[[4-(5-methyl-3-phenylisoxazol-4-yl)phenyl]sulfonyl]hexanamide;
3-methoxy-N-[[4-(5-methyl-3-phenylisoxazol-4-yl)phenyl[sulfonyl[propanamide;
2-ethoxy-N-[[4-(5-methyl-3-phenylisoxazol-4-yl)phenyl]sulfonyl]acetamide;
N-[[4-[5-methyl-3-phenylisoxazol-4-yl]phenyl]sulfonyl]acetamide;
N-[[4-[5-(4-chlorophenyl)-3-(trifluoromethyl)-1H pyrazol-1-yl]phenyl]sulfonyl]propanamide;
N-[[4-[5-(4-chlorophenyl)-3-(trifluoromethyl)-1H-pyrazol-1-yl]phenyl]sulfonyl]butanamide;
N-[[4-[5-(4-chlorophenyl)-3-(trifluoromethyl)-1H-pyrazol-1-yl]phenyl]sulfonyl]acetamide;
N-[[4-[3-(difluoromethyl)-6-fluoro-1,5-dihydro-7-methoxy-[2]benzothiopyrano[4,3-c]pyrazol-1-yl)phenyl]sulfonyl]acetamide;
N-[[4-[6-fluoro-1,5-dihydro-7-methoxy-3-(trifluoromethyl)-[2]benzothiopyrano[4,3-c]pyrazol-1-yl]phenyl]sulfonyl]acetamide;
N-[[4-[3-(difluoromethyl)-5-(3-fluoro-4-methoxyphenyl)-1H-pyrazol-1-yl]phenyl]sulfonyl]acetamide;
N-[[4-(2-methyl-4-phenyloxazol-5-yl)phenyl]sulfonyl]acetamide;
methyl[[[4-(5-methyl-3-phenylisoxazol-4-yl)phenyl]sulfonyl]amino]oxoacetate;
2-methoxy-N-[[4-(5-methyl-3-phenylisoxazol-4-yl)phenyl]sulfonyl]acetamide;
N-[[4-[5-(difluoromethyl)-3-phenylisoxazol-4-yl]phenyl]sulfonyl]propanamide;
N-[[4-[5-(difluoromethyl)-3-phenylisoxazol-4-yl]phenyl]sulfonyl]butanamide;
N-[[4-(5-methyl-3-phenylisoxazol-4-yl)phenyl]sulfonyl]formamide;
1,1-dimethylethyl-N-[[4-(5-methyl-3-phenylisoxazol-4-yl)phenyl]sulfonyl]carbamate;
N-[[.sup.4-(5-methyl-3-phenylisoxazol-4-yl)phenyl]sulfonyl]glycine;
2-amino-N-[[4-(5-methyl-3-phenylisoxazol-4-yl)phenyl]sulfonyl]acetamide;
2-(acetylamino)-N-[[4-(5-methyl-3-phenylisoxazol-4-yl)phenyl]sulfonyl]acetamide;
methyl 4-[[[4-(5-methyl-3-phenylisoxazol-4-yl)phenyl]sulfonyl]amino]-4-oxobutanoate;
methyl N-[[4-(5-methyl-3-phenylisoxazol-4-yl)phenyl]sulfonyl]carbamate;
N-acetyl-N-[[4-(5-methyl-3-phenylisoxazol-4-yl)phenyl]sulfonyl]glycine, ethyl ester;
N-[[4-(5-(4-methylphenyl)-3-(trifluoromethyl)-1H-pyrazol-1-yl)phenyl]sulfonyl]acetamide;
methyl 3-[[[4-(5-methyl-3-phenylisoxazol-4-yl)phenyl]sulfonyl]amino]-3-oxopropanoate;
4-[5-(3-bromo-5-fluoro-4-methoxyphenyl)-2-(trifluoromethyl)oxazol-4-yl]-N-methylbenezenesulfonamide;
N-(1,1-dimethylethyl)-4-(5-methyl-3-phenylisoxazol-4-yl)benzenesulfonamide;
4-[5-(4-fluorophenyl)-3-(trifluoromethyl)-1H-pyrazol-1-yl]-N-methylbenzenesulfonamide;
N-methyl-4-(5-methyl-3-phenylisoxazol-4-yl)benzenesulfonamide;
N-[[4-[5-(hydroxymethyl)-3-phenylisoxazol-4-yl]phenyl[sulfonyl]acetamide;
N-[[4-[5-(acetoxymethyl)-3-phenylisoxazol-4-yl]phenyl]sulfonyl]acetamide;
N-[[4-[2-(3-chloro-4-fluorophenyl)cyclopenten-1-yl)phenyl]sulfonyl]acetamide;
4-[2-(4-fluorophenyl)-1H-pyrrol-1-yl]-N-methylbenzenesulfonamide;
N-[[4-(3,4-dimethyl-1-phenyl-1H-pyrazol-5-yl]phenyl]sulfonyl]propanamide;
N-[[4-[2-(2-methylpyridin-3-yl)-4-trifluoromethylimidazol-1-yl]phenyl]sulfonyl]propanamide;
4-[2-(4-fluorophenyl)cyclopenten-1-yl]-N-methylbenezenesulfonamide; and
N-[[4-(3-phenyl-2,3-dihydro-2-oxofuran-4-yl)phenyl]sulfonyl]propanamide.

Prodrugs disclosed in U.S. Pat. No. 6,613,790 have formula (XXXIX) wherein:
A¹³ is a pyrazole group optionally substituted at a substitutable position with one or more radicals independently selected at each occurrence from the group consisting of alkylcarbonyl, formyl, halo, alkyl, haloalkyl, oxo, cyano, intro, carboxyl, alkoxy, aminocarbonyl, alkoxycarbonyl, carboxyalkyl, cyanoalkyl, hydroxyalkyl, haloalkylsulonyloxy, alkoxyalkyloxyalkyl, carboxyalkoxyalkyl, alkenyl, alkynyl, alkylthio, alkylthioalkyl, alkoxyalkyl, alkoxycarbonylalkyl, aminocarbonylalkyl, alkylaminocarbonyl, alkylaminocarbonylalkyl, alkylamino, aminoalkyl, alkylaminoalkyl, alkylsutfinyl, alkylsulfonyl, aminosulfonyl and alkylaminosulfonyl;
R²¹⁰ is a phenyl group optionally substituted at a substitutable position with one or more radicals independently selected at each occurrence from the group consisting of alkyl, haloalkyl, cyano, carboxyl, alkoxycarbonyl, hydroxyl, hydroxyalkyl, haloalkoxy, amino, alkylamino, nitro, alkoxyalkyl, alkylsulfinyl, halo, alkoxy, and alkylthio;
R²¹¹ and R²¹² are independently selected from the group consisting of hydroxyalkyl and hydrido but at least one of R²¹¹ and R²¹² is other than hydrido; and
R²¹³ is selected from the group consisting of hydrido and fluoro.

Specific non-limiting examples of substituted sulfonamide prodrugs of Cox-2 inhibitors disclosed in U.S. Pat. No. 6,613,790 that are useful in the present invention include:
N-(2-hydroxyethyl)-4-[5-(4-methylphenyl)-3-(trifluoromethyl)-1H-pyrazol-1-yl]benzenesulfonamide;
N,N-bis(2-hydroxyethyl)-4-[5-(4-methylphenyl)-3-(trifluoromethyl)-1H-pyrazol-1-yl]benzenesulfonamide;
   and pharmaceutically acceptable salts thereof.

Compounds that can act as Cox-2 selective inhibitors include sulfamoylheteroaryl pyrazole compounds as described in U.S. Pat. No. 6,583,321. Such compounds have the formula (XL): 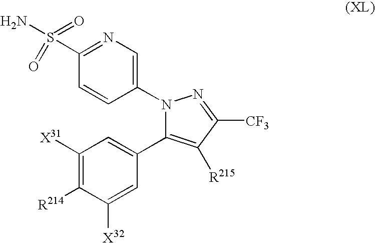
wherein:
R²¹⁴ is furyl, thiazolyl or oxazolyl;
R²¹⁵ is hydrogen, fluoro or ethyl; and
X³¹ and X³² are independently hydrogen or chloro.

Compounds that can act as Cox-2 selective inhibitors include heteroaryl substituted amidinyl and imidazolyl compounds as described in U.S. Pat. No. 6,555,563. Such compounds have the formula (XLI): 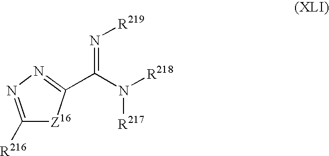
wherein:
Z¹⁶ is O or S;
R²¹⁶ is optionally substituted aryl;
R²¹⁷ is aryl optionally substituted with aminosulfonyl; and
R²¹⁸ and R²¹⁹ cooperate to form an optionally substituted 5-membered ring.

Compounds that can act as Cox-2 selective inhibitors include substituted hydroxamic acid derivatives as described in U.S. Pat. No. 6,432,999, U.S. Pat. No. 6,512,121, U.S. Pat. No. 6,515,014 and U.S. Pat. No. 6,555,563. These compounds also act as inhibitors of the lipoxygenase-5 enzyme. Such compounds have the formulas (XLII) and (XLIII):

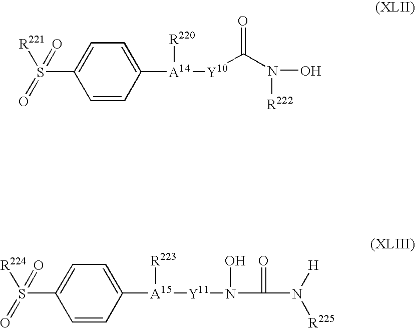

Pyrazole-substituted hydroxamic acid derivatives described in U.S. Pat. No. 6,432,999 can have formula (XLII), wherein:
A¹⁴ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl; and pharmaceutically acceptable salts thereof.

Pyrazole-substituted hydroxamic acid derivatives described in U.S. Pat. No. 6,432,999 can alternatively have formula (XLIII), wherein:
A¹⁵ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkylene, lower alkenylene and lower alkynylene;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²⁴ is selected from lower alkyl and amino; and
R²²⁵ is selected from hydrido and lower alkyl;
   and pharmaceutically acceptable salts thereof.

Heterocyclo-substituted hydroxamic acid derivatives described in U.S. Pat. No. 6,512,121 can have formula (XLII), wherein:
A¹⁴ is a ring substituent selected from oxazolyl, furyl, pyrrolyl, thiazolyl, imidazolyl, isothiazolyl, isoxazolyl, cyclopentenyl, phenyl, and pyridyl; wherein A¹⁴ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from lower alkylene, lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl;
   and pharmaceutically acceptable salts thereof.

Heterocyclo-substituted hydroxamic acid derivatives described in U.S. Pat. No. 6,512,121 can alternatively have formula (XLIII), wherein:
A¹⁵ is a ring substituent selected from oxazolyl, furyl, pyrrolyl, thiazolyl, imidazolyl, isothiazolyl, isoxazolyl, cyclopentenyl, phenyl, and pyridyl; wherein A¹⁵ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²⁴ is selected from lower alkyl and amino; and
R²²⁵ is selected from hydrido and alkyl;
   or a pharmaceutically-acceptable salt thereof.

Thiophene-substituted hydroxamic acid derivatives described in U.S. Pat. No. 6,515,014 can have formula (XLII), wherein:
A¹⁴ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from ethylene, isopropylene, propylene, butylene, lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl;
   and pharmaceutically acceptable salts thereof.

Thiophene substituted hydroxamic acid derivatives described in U.S. Pat. No. 6,515,014 can alternatively have formula (XLIII), wherein:
A¹⁵ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²⁴ is selected from lower alkyl and amino; and
R²²⁵ is selected from hydrido and alkyl;
   and pharmaceutically acceptable salts thereof.

Compounds that can act as Cox-2 selective inhibitors include pyrazolopyridine compounds as described in U.S. Pat. No. 6,498,166. Such compounds have the formula (XLIV): 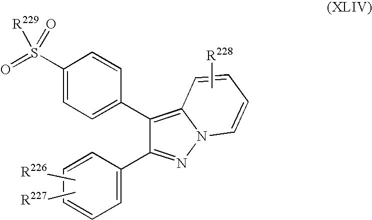
wherein:
R²¹⁶ and R²²⁷ are independently selected from the group consisting of H, halogen, C₁-C₆ alkyl, C₁-C₆ alkoxy, and C₁-C₆ alkoxy substituted by one or more fluorine atoms;
R²²⁸ is halogen, CN, CONR²³⁰R²³¹, CO₂H, CO₂(C₁-C₆ alkyl) or NHSO₂R²³⁰;
R²²⁹ is C₁-C₆ alkyl or NH₂; and
R²³⁰ and R²³¹ are independently selected from the group consisting of H, C₁-C₆ alkyl, phenyl, and phenyl substituted by one or more atoms or groups selected from the group consisting of halogen, C₁-C₆ alkyl, C₁-C₆ alkoxy, and C₁-C₆ alkoxy substituted by one or more fluorine atoms;
   or a pharmaceutically acceptable salt, solvate or ester thereof, or salt or solvate of such ester. Compounds that can act as Cox-2 selective inhibitors include 4,5-diaryl-3(2H)-furanone derivatives described in U.S. Pat. No. 6,492,416. Such compounds have the formula (XLV): 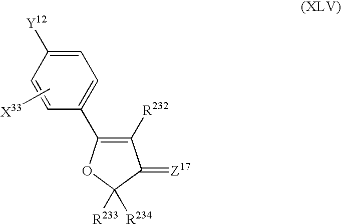
   wherein:
X³³ is halo, hydrido or alkyl;
Y¹² is alkylsulfonyl, aminosulfonyl, alkylsulfinyl, (N-acylamino)sulfonyl, (N-alkylamino)sulfonyl or alkylthio;
Z¹⁷ is an oxygen or sulfur atom;
R²³³ and R²³⁴ are selected independently from lower alkyl radicals; and
R²³² represents a substituted or non-substituted aromatic group of 5 to 10 atoms;
   and pharmaceutically acceptable salts thereof.

Compounds that can act as Cox-2 selective inhibitors include 2-phenyl-1,2-benzisoselenazol-3(2H)-one derivatives and 2-phenylcarbamylphenylselenyl derivatives as described in U.S. Pat. No. 6,492,416. Such compounds have the formulas (XLVI) and (XLVII): 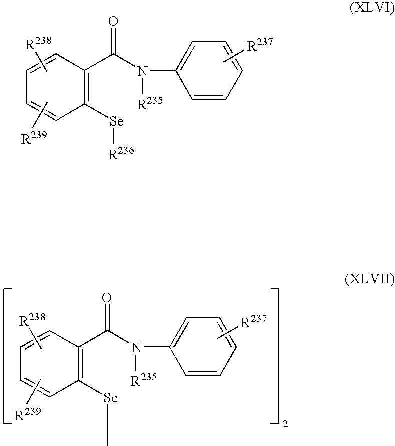
wherein:
R²³⁵ is a hydrogen atom or an alkyl group having 1-3 carbon atoms;
R²³⁶ is a hydrogen atom, a hydroxyl group, an organothiol group that is bound to the selenium atom by its sulfur atom, or R²³⁵ and R²³⁶ are joined to each other by a single bond;
R²³⁷ is a hydrogen atom, a halogen atom, an alkyl group having 1-3 carbon atoms, an alkoxy group having 1-3 carbon atoms, a trifluoromethyl group, or a nitro group;
R²³⁸ and R²³⁹ are identical to or different from each other, and each is a hydrogen atom, a halogen atom, an alkoxyl group having 1-4 carbon atoms, a trifluoromethyl group, or R²³⁸ and R²³⁹ are joined to each other to form a methylenedioxy group,
   and pharmaceutically acceptable salts thereof, and hydrates thereof.

Compounds that can act as Cox-2 selective inhibitors include pyrones as described in U.S. Pat. No. 6,465,509. Such compounds have the formula (XLVIII): 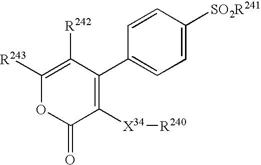
wherein:
X³⁴ is selected from the group consisting of a bond, —(CH₂)ₘ— wherein m is 1 or 2,—C(O)—, —O—, —S— and —N(R²⁴⁴)—;
R²⁴⁰ is selected from the group consisting of (a) C₁-C₁₀ alkyl, optionally substituted with 1-3 substituents independently selected from the group consisting of hydroxy, halo, C₁-C₁₀ alkoxy, C₁-C₁₀ alkylthio and CN, (b) phenyl, (c) naphthyl, and (d) heteroaryl comprising a monocyclic aromatic ring of 5 atoms having one hetero atom which is S, O or N, and optionally 1, 2 or 3 additional N atoms, or a monocyclic ring of 6 atoms having one hetero atom which is N, and optionally 1, 2 or 3 additional N atoms; wherein groups (b), (c) and (d) are optionally substituted with 1-3 substituents independently selected from the group consisting of halo, C₁-C₁₀ alkoxy, C₁-C₁₀ alkylthio, CN, C₁-C₁₀ alkyl optionally substituted to its maximum with halo, and N₃;
R²⁴¹ is selected from the group consisting of (a) C₁-C₆ alkyl optionally substituted to its maximum with halo, (b) NH₂, and (c) NHC(O)(C₁-C₁₀ alkyl) optionally substituted to its maximum with halo;
R²⁴² and R²⁴³ are independently selected from the group consisting of hydrogen, halo and C₁-C₆ alkyl optionally substituted to its maximum with halo; and
R²⁴⁴ is selected from the group consisting of hydrogen and C₁-C₆ alkyl optionally substituted to its maximum with halo.

Examples of pyrone compounds that are useful as Cox-2 selective inhibitors of the present invention include, but are not limited to:
4-(4-methylsulfonyl)phenyl-3-phenyl-pyran-2-one;
3-(4-fluorophenyl)-6-methyl-4-(4-methylsulfonyl)phenyl-pyran-2-one;
3-(3-fluorophenyl)-6-methyl-4-(4-methylsulfonyl)phenyl-pyran-2-one;
6-methyl-4-(4-methylsulfonyl)phenyl-3-phenyl-pyran-2-one;
6-difluoromethyl-4-(4-methylsulfonyl)phenyl-3-phenyl-pyran-2-one;
6-fluoromethyl-4-(4-methylsulfonyl)phenyl-3-phenyl-pyran-2-one;
6-methyl-4-(4-methylsulfonyl)phenyl-3-phenylthio-pyran-2-one;
6-methyl-4-(4-methylsulfonyl)phenyl-3-phenoxy-pyran-2-one;
6-methyl-4-(4-methylsulfonyl)phenyl-3-pyridin-3-yl-pyran-2-one;
3-isopropylthio-6-methyl-4-(4-methylsulfonyl)phenyl-pyran-2-one;
4-(4-methylsulfonyl)phenyl)-3-phenylthio-6-trifluoromethyl-pyran-2-one;
3-isopropylthio-4-(4-methylsulfonyl)phenyl-6-trifluoromethyl-pyran-2-one;
4-(4-methylsulfonyl)phenyl-3-phenyl-6-(2,2,2-trifluoroethyl)-pyran-2-one; and
3-(3-hydroxy-3-methylbutyl)-6-methyl-4-(4-methylsulfonyl)phenyl-pyran-2-one.

Compounds that can act as Cox-2 selective inhibitors include free-B-ring flavonoids as described in U.S. patent application Publication No. 2003/0165588. Such compounds, organically synthesized or purified from plant sources, have the formula (XLIX): 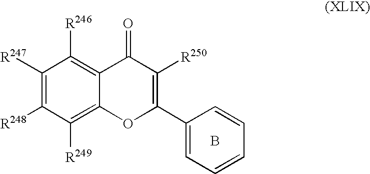
wherein R²⁴⁶, R²⁴⁷, R²⁴⁸, R²⁴⁹ and R²⁵⁰ are independently selected from the group consisting of —H, —OH, —SH, —OR, —SR, —NH₂, —NHR²⁴⁵, —N(R²⁴⁵)₂, —N(R²⁴⁵)₃⁺X³⁵⁻, a carbon, oxygen, nitrogen or sulfur glycoside of a single or a combination of multiple sugars selected from aldopentoses, methyl-aldopentose, aldohexoses, ketohexose and chemical derivatives thereof; where R²⁴⁵ is an alkyl group having 1-10 carbon atoms, and X³⁵ is selected from the group of pharmaceutically acceptable counter-anions consisting of hydroxyl, chloride, iodide, sulfate, phosphate, acetate, fluoride and carbonate.

Compounds that can act as Cox-2 selective inhibitors include heterocyclo-alkylsulfonyl pyrazoles as described in European Patent Publication No. EP 1 312 367. Such compounds have the formula (L): 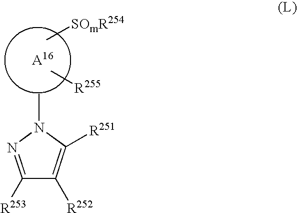
wherein:
ring A¹⁶ is selected from the group consisting of 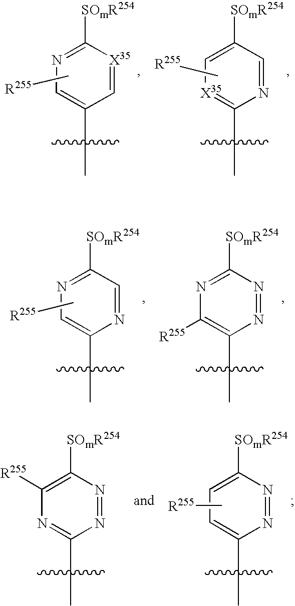
m is 0, 1 or 2;
X³⁵ is >CR²⁵⁵ or >N;
R²⁵¹ is a radical selected from the group consisting of H, NO₂, CN, C₁-C₆ alkyl, (C₁-C₆ alkyl)-SO₂—, (C₆-C₁₀ aryl)-SO₂, H—(C═O)—, (C₁-C₆ alkyl)-(C═O)—, (C₁-C₆ alkyl)-(C═O)—, (C₁-C₉ heteroaryl)-(C═O)—, (C₁-C₉ heterocyclyl)-(C═O)—, H₂N—(C═O)—, (C₁-C₆ alkyl)-NH—(C═O)—, (C₁-C₆ alkyl)₂-N—(C═O)—, (C₆-C₁₀ aryl)₂-NH—(C═O)—, (C₁-C₆ alkyl)-[(C₆-C₁₀ aryl)-N[—(C═O)—, HO—NH—(C═O)— and (C₁-C₆ alkyl)-O—NH-(C═O)—;
R²⁵² is a radical selected from the group consisting of H, NO₂, CN, C₂-C₆ alkenyl, C₂-C₆ alkynyl, C₃ -C₇ cycloalkyl, C₆-C₁₀ aryl, C₁-C₉ heteroaryl, C₁-C₉ heterocyclyl, (C₁-C₆ alkyl)-O—, (C₃-C₇ cycloalkyl)-O—, (C₆-C₁₀ aryl)-O—, (C₁-C₉ heteroaryl)-O—, (C₆-C₉ heterocyclyl)-O—, H—(C═O)—, (C₁-C₆ alkyl)-(C═O)—, (C₃-C₇ cycloalkyl)-(C═O)—, (C₆-C₁₀ aryl)-(C═O)—, (C₁-C₉ heteroaryl)-(C═O)—, (C₁-C₉ heterocyclyl)-(C═O)—, (C₁-C₆ alkyl)-O—(C═O)—, (C₃-C₇ cycloalkyl)-O—(C═O)—, (C₆-C₁₀ aryl)-O—(C═O)—, (C₁-C₉ heteroaryl)-O—(C═O)—, (C₁-C₉ heterocyclyl)-O—(C═O)—, (C₁-C₆ alkyl)-(C═O)—O—, (C₃-C₇ cycloalkyl)-(C═O)—O—, (C₆-C₁₀ aryl)-(C═O)—O—, (C₁-C₉ heteroaryl)-(C═O)—O—, (C₁-C₉ heterocyclyl)-(C═O)—O—, (C₁-C₆ alkyl)-(C═O)—NH—, (C₃-C₇ cycloalkyl)-(C═O)—NH—, (C₆-C₁₀ aryl)-(C═O)—NH—, (C₁-C₉ heteroaryl)-(C═O)—NH—, (C₁-C₉ heterocyclyl)-(C═O)—NH—, (C₁-C₆ alkyl)-O—(C═O)—NH—, (C₁-C₆ alkyl)-NH, (C₁-C₆ alkyl)₂-N—, (C₃-C₇ cycloalkyl)-NH—, (C₃-C₇ cycloalkyl)₂-N—, (C₆-C₁₀ aryl)-NH—, (C₆-C₁₀ aryl)₂-N—, (C₁-C₆ alkyl)-[(C₆-C₁₀ aryl)-N]—, (C₁-C₉ heteroaryl)-NH—, (C₁-C₉ heteroaryl)₂-N—, (C₁-C₉ heterocyclyl)-NH—, (C₁-C₉ heterocyclyl)₂-N—, H₂N—(C═O)—, HO—NH—(C═O)—, (C₁-C₆ alkyl)-O—NH—(C═O)—, (C₁-C₆ alkyl)-NH—(C═O)—, (C₁-C₆ alkyl)₂-N—(C═O)—, (C₃-C₇ cycloalkyl)-NH—(C═O)—, (C₃-C₇ cycloalkyl)₂-N—(C═O)—, (C₆-C₁₀ aryl)-NH—(C═O)—, (C₆-C₁₀ aryl)₂-N—(C═O)—, (C₁-C₆ alkyl)-[(C₆-C₁₀ aryl)-N]—(C═O)—, (C₁-C₉ heteroaryl)-NH—(C═O)—, (C₁-C₉ heteroaryl)₂-N—(C═O)—, (C₁-C₉ heterocyclyl)-NH—(C═O)—, (C₁-C₆ alkyl)-S— and C₁-C₆ alkyl optionally substituted by one —OH substituent or by one to four fluoro substituents;
R²⁵³ is a saturated 3- to 4-membered heterocyclyl ring radical; or a saturated, partially saturated or aromatic 7- to 9-membered heterocyclyl ring radical; wherein said ring radical (a) optionally contains one to four ring heteroatoms independently selected from the group consisting of —N═, —NH—, —O— and —S—; (b) optionally is substituted on any ring carbon atom by one to three substituents per ring independently selected from the group consisting of halo, OH, CN, NO₂, C₂-C₆ alkenyl, C₂-C₆ alkynyl, C₃-C₇ cycloalkyl, C₆-C₁₀ aryl, C₂-C₉ heterocyclyl, (C₁-C₆ alkyl)-O—, H—(C═O)—, (C₁-C₆ alkyl)-(C═O)—, HO—(C═O)—, (C₁-C₆ alkyl)-O—(C═O)—, —NH₂, (C₁-C₆ alkyl)-NH—, (C₁-C₆ alkyl)₂-N—, (C₃-C₇ cycloalkyl)-NH—, (C₆-C₁₀ aryl)-NH—, (C₁-C₆ alkyl)-[(C₆-C₁₀ aryl)-N]—, (C₁-C₉ heteroaryl)-NH—, H₂N—(C═O)—(C₁-C₆ alkyl)-NH—(C═O)—, (C₁-C₆ alkyl)₂-N—(C═O)—, (C₆-C₁₀ aryl)—NH—(C═O)—, (C₁-C₆ alkyl)-[(C₆-C₁₀ aryl)-N]—(C═O)—, (C₁-C₆ alkyl)-O—NH—(C═O)—, (C₁-C₆ alkyl)—(C═O)—HN—, (C₁-C₆ alkyl)-(C═O)—[(C₁-C₆ alkyl)-N]—, —SH, (C₁-C₆ alkyl)-S—, (C₁-C₆ alkyl)-(S═O)—, (C₁-C₆ alkyl)—SO₂—, and C₁-C₆; alkyl optionally substituted with one to four fluoro moieties; and (c) optionally is substituted on any ring nitrogen atom by one to three substituents per ring independently selected from the group consisting of C₃-C₇ cycloalkyl, C₆-C₁₀ aryl, C₂-C₉ heterocyclyl, H—(C═O)—, (C₁-C₆ alkyl)-(C═O)—, (C₁-C₆ alkyl)-O—(C═O)—, H₂N—(C═O)—, (C₁-C₆ alkyl)-NH-(C═O)—, (C₁-C₆ alkyl)₂-N—(C═O)—, (C₆-C₁₀ aryl)-NH—(C═O)—, (C₁-C₆ alkyl)-[(C₆-C₁₀ aryl)-N]—(C═O)—, (C₁-C₆ alkyl)-O—NH—(C═O)—, and C₁-C₆ alkyl optionally substituted with one to four fluoro moieties;
R²⁵⁴ is a C₁-C₆ alkyl radical optionally substituted by one to four fluoro substituents; and
R²⁵⁵ is a radical selected from the group consisting of H, halo, OH, (C₁-C₆ alkyl)-O—, C₂-C₆ alkenyl, C₂-C₆ alkynyl, C₃-C₇ cycloalkyl, CN, H—(C═O)—, (C₁-C₆ alkyl-(C═O)—, (C₁-C₆ alkyl)-(C═O)—O—, HO—(C═O)—, (C₁-C₆ alkyl)-O—(C═O)—, (C₁-C₆ alkyl)-NH—, (C₁-C₆ alkyl)₂-N—, (C₃-C₇ cycloalkyl)-NH—, (C₆-C₁₀ aryl)-NH—, (C₁-C₆ alkyl)-[(C₆-C₁₀ aryl)-N]—, (C₁-C₉ heteroaryl)-NH—, H₂N—(C═O)—, (C₁-C₆ alkyl)-N—-(C═O)—, (C₁-C₆ alkyl)₂-N—(C═O)—, (C₆-C₁₀ aryl)-(C═O)—, (C₁-C₆ alkyl)-[(C₆-C₁₀ aryl)-N]—(C═O)—, (C₁-C₆ alkyl-O—NH—(C═O)—, (C₁-C₆ alkyl)-S—, and C₁-C₆ alkyl optionally substituted by one to four fluoro substituents;
   and pharmaceutically acceptable salts thereof.

Compounds that can act as Cox-2 selective inhibitors include 2-phenylpyran-4-one derivatives as described in U.S. Pat. No. 6,518,303. Such compounds have the formula (LI): 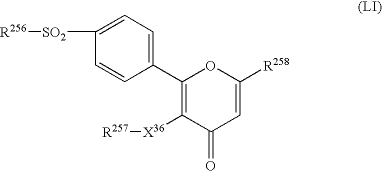
wherein:
R²⁵⁶ is an alkyl or —NR²⁵⁹R²⁶⁰ group, where R²⁵⁹ and R²⁶⁰ are independently selected from a hydrogen atom and an alkyl group;
R²⁵⁷ is an alkyl, C₃-C7 cycloalkyl, naphthyl, tetrahydronaphthyl or indanyl group, or a phenyl group which is unsubstituted or substituted by one or more halogen atoms or alkyl, trifluoromethyl, hydroxy, alkoxy, methylthio, amino, mono- or dialkylamino, hydroxyalkyl or hydroxycarbonyl groups;
R²⁵⁸ is a methyl, hydroxymethyl, alkoxymethyl, C₃-C₇ cycloalkoxymethyl, benzyloxymethyl, hydroxycarbonyl, nitrile, trifluoromethyl or difluoromethyl group or a CH₂—R²⁶¹ group where R²⁶¹ is an alkyl group; and
X³⁶ is a single bond, an oxygen atom, a sulfur atom or a methylene group;
   and pharmaceutically acceptable salts thereof.

Examples of 2-phenylpyran-4-one derivatives useful in the present invention include, but are not limited to:
3-(4-fluorophenyl)-2-(4-methanesulfonylphenyl)-6-methylpyran-4-one;
3-(2-fluorophenyl)-2-(4-methanesulfonylphenyl)-6-methylpyran-4-one;
3-(4-chlorophenyl)-2-(4-methanesulfonylphenyl)-6-methylpyran-4-one;
3-(4-bromophenyl)-2-(4-methylsulfonylphenyl)-6-methylpyran-4-one;
3-(2,4-difluorophenyl)-2-(4-methanesulfonylphenyl)-6-methylpyran-4-one;
3-(3,4-dichlorophenyl)-2-(4-methanesulfonylphenyl)-6-methylpyran-4-one;
3-(3-chloro-4-methylphenyl)-2-(4-methanesulfonylphenyl)-6-methylpyran-4-one;
2-(4-methanesulfonylphenyl)-6-methyl-3-phenoxypyran-4-one;
3-(4-fluorophenoxy)-2-(4-methanesulfonylphenyl)-6-methylpyran-4-one;
3-(2-fluorophenoxy)-2-(methanesulfonylphenyl)-6-methylpyran-4-one;
3-(4-chlorophenoxy)-2-(methanesulfonylphenyl)-6-methylpyran-4-one;
3-(2-chlorophenoxy)-2-(methanesulfonylphenyl)-6-methylpyran-4-one;
3-(4-bromophenoxy)-2-(4-methanesulfonylphenyl)-6-methylpyran-4-one;
2-(4-methanesulfonylphenyl)-6-methyl-3-(4-methylphenoxy)pyran-4-one;
3-(2,4-difluorophenoxy)-2-(4-methanesulfonylphenyl)-6-methylpyran-4-one;
3-(2,5-difluorophenoxy)-2-(methanesulfonylphenyl)-6-methylpyran-4-one;
3-(4-chlorophenyl)-2-(4-methanesulfonylphenyl)-6-methoxymethylpyran-4-one;
3-(4-chlorophenyl)-6-difluoromethyl-2-(4-methanesulfonylphenyl)pyran-4-one;
   and pharmaceutically acceptable salts thereof.

Cox-2 selective inhibitors useful in the subject methods and compositions can include compounds described in the patents individually cited below and incorporated herein by reference.

U.S. Pat. No. 6,472,416.

U.S. Pat. No. 6,451,794.

U.S. Pat. No. 6,169,188.

U.S. Pat. No. 6,020,343.

U.S. Pat. No. 5,981,576.

U.S. Pat. No. 6,222,048.

U.S. Pat. No. 6,057,319.

U.S. Pat. No. 6,046,236.

U.S. Pat. No. 6,002,014.

U.S. Pat. No. 5,945,539.

U.S. Pat. No. 6,359,182.

U.S. Pat. No. 6,538,116.

Cox-2 selective inhibitors useful in the present invention can be supplied by any source as long as the Cox-2 selective inhibitor is pharmaceutically acceptable. Cox-2 selective inhibitors can be isolated and purified from natural sources or can be synthesized. Cox-2 selective inhibitors should be of a quality and purity that is conventional in the trade for use in pharmaceutical products.

Celecoxib useful in the combinations, method, kits and compositions of the invention can be prepared, for example, as set forth in U.S. Pat. No. 5,466,823.

Valdecoxib useful in the combinations, method, kits and compositions of the invention can be prepared, for example, as set forth in U.S. Pat. No. 5,633,272.

Parecoxib useful in the combinations, method, kits and compositions of the invention can be prepared, for example, as set forth in U.S. Pat. No. 5,932,598.

Rofecoxib useful in the combinations, method, kits and compositions of the invention can be prepared, for example, as set forth in U.S. Pat. No. 5,968,974.

Japan Tobacco JTE-522 useful in the combinations, method, kits and compositions of the invention can be prepared, for example, as set forth in Japanese Patent Publication No. JP 90/52882.

Pyrazoles useful in the combinations, method, kits and compositions of the invention can be prepared, for example, as set forth in International Patent Publication No. WO 95/15316.

Pyrazoles can also be prepared as set forth in International Patent Publication No. WO 95/15315.

Pyrazoles can also be prepared as set forth in International Patent Publication No. WO 96/03385.

Thiophene analogs useful in the combinations, method, kits and compositions of the invention can be prepared, for example, as set forth in International Patent Publication No. WO 95/00501.

Thiophene analogs can also be prepared as set forth in International Patent Publication No. WO 94/15932.

Oxazoles useful in the combinations, method, kits and compositions of the invention can be prepared, for example, as set forth in International Patent Publication No. WO 95/00501.

Oxazoles can also be prepared as set forth in International Patent Publication No. WO 94/27980.

Isoxazoles useful in the combinations, method, kits and compositions of the invention can be prepared, for example, as set forth in International Patent Publication No. WO 96/25405.

Imidazoles useful in the combinations, method, kits and compositions of the invention can be prepared, for example, as set forth in International Patent Publication No. WO 96/03388.

Imidazoles can also be prepared as set forth in International Patent Publication No. WO 96/03387.

Cyclopentene Cox-2 inhibitors useful in the combinations, method, kits and compositions of the invention can be prepared, for example, as set forth in U.S. Pat. No. 5,344,991.

Cyclopentene Cox-2 inhibitors can also be prepared as set forth in International Patent Publication No. WO 95/00501.

Terphenyl compounds useful in the combinations, method, kits and compositions of the invention can be prepared, for example, as set forth in International Patent Publication No. WO 96/16934.

Thiazole compounds useful in the combinations, method, kits and compositions of the invention can be prepared, for example, as set forth in International Patent Publication No. WO 96/03,392.

Pyridine compounds useful in the combinations, method, kits and compositions of the invention can be prepared, for example, as set forth in International Patent Publication No. WO 96/03392.

Pyridine compounds can also be prepared as set forth in International Patent Publication No. WO 96/24585.

Illustratively, a Cox-2 selective inhibitor can be a tricyclic compound, for example a compound of formula (VII), a substituted benzopyran derivative, for example a compound of formulas (I) to (VI), or a phenylacetic acid derivative, for example a compound of formula (VIII).

Illustratively, the Cox-2 selective inhibitor can be selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

More particularly, the Cox-2 selective inhibitor can be selected from the group consisting of celecoxib, valdecoxib, parecoxib, rofecoxib, etoricoxib, lumiracoxib, and pharmaceutically acceptable salts thereof.

In one embodiment the Cox-2 selective inhibitor comprises celecoxib.

In another embodiment the Cox-2 selective inhibitor comprises valdecoxib.

In yet another embodiment the Cox-2 selective inhibitor comprises parecoxib sodium.

In certain embodiments, the Cox-2 selective inhibitor is selected from compounds of formulas (XXXVII) to (LI) hereinabove.

A second component of the methods and compositions of the present invention is a DNA topoisomerase I inhibitor.

As used herein, the terms “DNA topoisomerase I inhibitor”, “topoisomerase I inhibitor”, and topoisomerase inhibitor”, used interchangeably herein, include any compound that inhibits, disrupts or degrades the activity of the DNA topoisomerase I protein by disrupting the enzyme/substrate complex or by interfering with the synthesis of the DNA topoisomerase I protein itself. In one embodiment, the compound inhibits DNA topoisomerase I through direct contact. In specific embodiments, the contact is at a singular point. In other embodiments, the contact is through multiple and distinct contacts with residues in the protein.

The DNA topoisomerase I inhibitory effect of a compound of unknown inhibitory activity can be assessed, for example, by monitoring DNA cleavage activity according to standard techniques. In one embodiment, one of skill in the art will understand how to determine if an unknown compound is a DNA topoisomerase I inhibitor by employing the supercoiled pBR 322 DNA relaxation assay, following the disclosure of Liu et al., Proc. Natl. Acad. Sci., 76:3487 (1987).

DNA topoisomerase I inhibiting agents of particular interest that can be used with the methods, combinations and compositions of the present invention are provided in Table 3. The therapeutic compounds of Table 3 can be used in the methods, combinations and compositions of the present invention in a variety of forms, including acid form, salt form, racemates, enantiomers, zwitterions, and tautomers.

TABLE 3
DNA topoisomerase I inhibitors

| | Trade | | | | |
|---|---|---|---|---|---|
| Compound Name | Name | Reference | Dosage | Toxicity | Oncology Indication |
| | Campto- | WO 9637496 | | myelosuppression, | Colon, stomach, and non- |
| | thecin | J. Am. Chem. Soc. | | nausea, vomiting, and | small cell lung cancer. |
| | | 1966; 88: 3888-90. | | diarrhea, and | Melanoma. |
| | | | | hemorrhagic cystitis. | |
| 9-amino-20(S)- | | Cancer Res. 1989; | | | Colon, non-small cell lung, |
| camptothecin | | 49: 1465-1469. | | | and breast cancer. |
| | | Cancer Res. 1989; | | | Melanoma. |
| | | 49: 4385-4389 | | | |
| | GG211 | Proc Am Assoc. Cancer | | Hematologic toxicity | Colon, ovarian, lung and |
| | | Res. 1994; 35: 47. | | dose limiting. | epidermoid cancer. |
| | Irino- | Cancer Res. 1991; | 20 mg/m² for 3 days | Diarrhea and myelosup- | Colon, head and neck, non- |
| | tecan | 51: 4187-4191. | weekly; | pression. | small cell lung, cervical, |
| | | Cancer Res. 1987; | 100 mg/m² weekly; | | esophageal, renal cell, |
| | | 47: 5944-5947. | 150 mg/m² every 2 | | breast, and ovarian cancer. |
| | | Cancer Res. 1990; | weeks; 200 mg/m² | | Gastric and lung squamous |
| | | 50: 1715-1720. | every 3-4 weeks; | | cell carcinomas. |
| | | | 250 mg/m² every 3- | | Rhabdomysarcoma. Non- |
| | | | 4 weeks. | | Hodgkin's lymphoma. |
| | | | | | Combination therapy: |
| | | | | | Recombinant granulocyte |
| | | | | | colony stimulating factor |
| | | | | | (G-CSF). 5-fluorouracil. |
| | | | | | Cisplatin Etoposide |
| (4S)-4,11-diethyl-4- | Irino- | U.S. Pat. No. 4604463. | 125 mg/m² IV over | Lethality in mice: 111 | Metastatic carcinoma of |
| hydroxy-9-((4-piperi- | tecan | EP 56692. | 90 minutes/wk for 4 | mg/kg in mice. Lethality | the colon or rectum. Brain |
| dinopiperidino)carbonyl | hydro- | JP 60019790. | weeks followed by | in rats: 73 mg/kg. DLT: | tumor, arcinoma, Lung |
| oxy)-1H- | chloride, | | 2 week rest. Then | diarrhea and | tumor, Neoplasm, Non- |
| pyrano(3′,4′:6,7)indoli- | CPT-11. | | repeated at 50 to | neutropenia. Myelosup- | Hodgkin lymphoma, Non- |
| zino(1,2-b)quinoline- | Camptosar ® | | 150 mg/m² doses. | pression, neutropenia, | small-cell lung cancer, |
| 3,14(4H,12H)dione | Injection | | | leukopenia (including | Ovary tumor, Pancreas |
| hydrochloride. | | | | lympho-cytopenia), and | tumor, Stomach tumor, |
| | | | | anemia. | Uterine cervix tumor, |
| | | | | | Uterus tumor. |
| (S)-10- | Topotecan | | 1.5 mg/m²/d IV | DLT: Bone marrow sup- | Metastatic carcinoma of |
| ((dimethylamino)methyl)- | hydro- | | infusion over 30 | pression. LD10: mice 75 | the ovary. |
| 4-ethyl-4,9-dihydroxy- | chloride; | | minutes for 5 con- | mg/m² single IV | Radio/chemosensitizer; |
| 1H-pyrano (3′, | Hycamtin | | secutive days, | infusion. Grade 4 | Breast tumor, Carcinoma, |
| 4′:6,7)indolizino(1,2- | | | starting on day one | thrombo-cytopenia, | Colon tumor, Glioma, |
| B)quinoline-3,14- | | | of a 21-day course. | anemia. | Leukemia, Lung tumor, |
| (4H,12H)-dione | | | | | Lymphoma, |
| monohydrochloride | | | | | Myeloproliferative |
| | | | | | disorder. |
| 1H- | Topotecan | EP 321122. | 1.5 mg/m² × 5 d | Maximally tolerated | Colorectal, small and non- |
| Pyrano[3′,4′:6,7]indoli- | | | every 3 wk: | dose: 1.5 mg/m² × 5 d | small cell lung cancer; |
| zino[1,2-b]quinoline- | | | Prostate, colorectal, | every 3 to 4 wk. | ovarian, esophageal, renal, |
| 3,14(4,H,12H)-dione, | | | and ovarian cancer. | Myelosup-pression dose- | squamous cell skin, |
| 10-[(dimethylamino)meth- | | | 1.5 mg/m² × 5 d | limiting toxicity. | prostate, and epidermoid |
| yl]-4-ethyl-4,9- | | | every 4 wk: Renal | Subsequent administra- | cancer. Osteogenic |
| dihydroxy-, (S)- | | | cell cancer. | tion of G-CSF lowers | sarcoma, rhabdo- |
| | | | | severity of neutropenia, | mysarcoma, acute myelo- |
| | | | | allowing dose escaltion. | blastic leukemia, chronic |
| | | | | | myelocytic leukemia in |
| | | | | | blastic phase. Leiomyo- |
| | | | | | sarcoma. Combination |
| | | | | | therapy: Etoposide and |
| | | | | | cisplatin. |
| MAG-camptothecin | PNU-166148 | Proc Am Soc. Clin Oncol | | | Solid tumors, |
| (prodrug) | | 2000 19 May 20-23 Abs | | | |
| | | 771 | | | |
| 11H-1,4-Dioxino[2,3- | lurtotecan | EP 540099 | 0.3 to 0.5 | hematological toxicity, | neoplasia |
| g]pyrano[3′,4′:6,7]indoli- | | | mg/m2/day by | myelotoxicity, | |
| zino[1,2-b]quinoline- | | | continuous | gastrointestinal toxicity, | |
| 9,12(8H,14H)-dione, 8- | | | infusions of 7, 14, | thrombocytopenia and | |
| ethyl-2,3-dihydro-8- | | | and 21 days. | neutropenia | |
| hydroxy-15-[(4-methyl- | | | | and asthenia | |
| 1-piperazinyl)methyl]-, | | | | | |
| (S)- | | | | | |
| 11H-1,4-Dioxino[2,3- | Lurtotecan | EP 540099 | 0.3 to 0.5 | hematological toxicity, | neoplasia |
| g]pyrano[3′,4′:6,7]indoli- | dihydro- | | mg/m2/day by | myelotoxicity, | |
| zino[1,2-b]quinoline- | chloride | | continuous | gastrointestinal toxicity, | |
| 9,12(8H,14H)-dione, 8- | | | infusions of 7, 14, | thrombocytopenia and | |
| ethyl-2,3-dihydro-8- | | | and 21 days. | neutropenia and asthenia | |
| hydroxy-15-[(4-methyl- | | | | | |
| 1-piperazinyl)methyl]-, | | | | | |
| dihydrochloride, (S)- | | | | | |
| 1H- | 9-amino- | | Dose limiting | Maximum tolerated dose = | Colon tumor, Solid tumor, |
| Pyrano[3′,4′:6,7]indoli- | campto- | | toxicity consisted of | 45 mug/square | Neoplasm, Carcinoma, |
| zino[1,2-b]quinoline- | thecin | | neutropenia. | metre/hr; | Lung tumor, Colorectal |
| 3,14(4H,12H)-dione, 10- | | | | | tumor, Pancreas tumor, |
| amino-4-ethyl-4- | | | | | Stomach tumor, Bladder |
| hydroxy-, (S)- | | | | | tumor, Prostate tumor, |
| | | | | | Head & neck tumor, Renal |
| | | | | | tumor, Leukemia |
| | DB-67, | WO 99/09996 | | | Neoplasia |
| | campto- | | | | |
| | thecins, | | | | |
| | homosila- | | | | |
| | tecans | | | | |
| 1H- | rubitecan, | Eur J Haematol 1994 53 | Maximum tolerated | The dose limiting | Neoplasm, Pancreas tumor, |
| Pyrano[3′,4′:6,7]indoli- | 9- | 4 246-248. | dose: 1.5 | toxicity was | Ovary tumor, Leukemia, |
| zino[1,2-b]quinoline- | nitrocamp- | Proc Am Assoc. Cancer | mg/m2/day over | hematological, with | Solid tumor, |
| 3,14(4H,12H)-dione, -4- | tothecin | Res. 1994 35 Abs 2712. | five consecutive | grade 4 anemia in 29% | Myelodysplastic Disease |
| ethyl-4-hydroxy-10- | | Int J Cancer 1993 53 5 | days repeated every | of patients, neutropenia | |
| nitro-, (S)- | | 863-871. | week. | in 25%, and | |
| | | | | thrombocytopenia in | |
| | | | | 18%. Grade 2 or higher | |
| | | | | toxic effects occurred at | |
| | | | | each dose level: nausea | |
| | | | | and vomiting (54%), | |
| | | | | diarrhea (32%), chemical | |
| | | | | cystitis (25%), | |
| | | | | neutropenic sepsis | |
| | | | | (21%), and weight loss | |
| | | | | (18%). | |
| 7-[N-(4-methyl-1- | CT-17 | Proc Am Assoc. Cancer | | | Neoplasia |
| piperazino) | | Res. 1999 40 ABS 715 | | | |
| methylamino]-(20S)- | | | | | |
| camptothecin | | | | | |
| camptothecin | BAY-38- | Clin Cancer Res. 1999 5 | | | Neoplasia |
| glycoconjugates | 3441 | 11 3862s-3863s. | | | |
| | | Proc Am Assoc. Cancer | | | |
| | | Res. 2000 41 April 1-5 | | | |
| | | Abs 3430. | | | |
| camptothecin | BAY-38- | Clin Cancer Res. 1999 5 | | | Neoplasia |
| glycoconjugates | 3444 | 11 3862s-3863s. | | | |
| 4(3H)-Quinazolinone, | NSC-665517 | Proc Am Assoc. Cancer | | | Carcinoma |
| 6,8-dibromo-2-methyl-3- | | Res. 1995 36 Abs 2659. | | | |
| [2-(D- | | Mol Pharmacol 1995 48 | | | |
| xylopyranosylamino)phen- | | 4 658-665 | | | |
| yl]- | | | | | |
| 2-Propenamide, 2- | AG 490, | | | | Neoplasia |
| cyano-3-(3,4- | Tyrphostin | | | | |
| dihydroxyphenyl)-N- | AG 490 | | | | |
| (phenylmethyl)-, (2E)- | | | | | |
| - | | | | | |
| 2-Propenamide, 2- | AG 555, | Cancer Res. 1994 54 19 | | | Neoplasia |
| cyano-3-(3,4- | Tyrphostin | 5138-5142. | | | |
| dihydroxyphenyl)-N-(3- | AG 555 | Exp Opin Ther Pat 1998 | | | |
| hydroxyphenylpropyl)-, | | 8 12 1599-1625 | | | |
| (E)- | | | | | |
| | NSC-314622 | Proc Am Assoc. Cancer | | | Neoplasia |
| | | Res. 1996 431. | | | |
| | | Proc Am Assoc. Cancer | | | |
| | | Res. 2000 41 April 1-5 | | | |
| | | Abs 5186. | | | |
| | CZ-112; | U.S. Pat. No. 5731316 | | | malignant tumors, |
| | CZ-48 | | | | neoplasia |
| | HAR-7 | Nci Eortc Symp New | | | Solid tumors |
| | | Drugs Cancer Ther 1996 | | | |
| | | 9th Amsterdam Abs 444. | | | |
| | NX-211, | Proc Am Assoc. Cancer | | | Neoplasia |
| | lurtotecan | Res. 1999 40 Abs 751. | | | |
| | liposomal | Proc Am Soc. Clin Oncol | | | |
| | | 1999 18 15-18 May 680. | | | |
| 5H-Indolo[2,3- | J 107088; | Proc Am Assoc. Cancer | maximum tolerated | | Neoplasia |
| a]pyrrolo[3,4- | ED-749 | Res. 1998 39 New | dose: 7.5 mg/m2 | | |
| c]carbazole-5,7(6H)- | | Orleans Abs 2864. | | | |
| dione, 12-.beta.-D- | | Ann Oncol 1998 9 2 043. | | | |
| glucopyranosyl-12,13-di- | | Cancer Res. 1999 59 17 | | | |
| hydro-2,10-dihydroxy-6- | | 4271-4275. Bioorg Med | | | |
| [[2-hydroxy-1- | | Chem. Lett 1999 9 23 | | | |
| (hydroxymethyl)ethyl]a- | | 3307-3312. | | | |
| mino]- | | | | | |
| 4-Acridinecarboxamide, | XR-5000, | U.S. Pat. No. 05696131. | | infusion-related arm pain | Brain tumor, Breast tumor, |
| N-[2- | DACA | Journal Of Medicinal | | | Carcinoma, Colon tumor, |
| (dimethylamino)ethyl]-, | | Chemistry 1987 30 664- | | | Lung tumor, Melanoma, |
| dihydrochloride | | 669 | | | Ovary tumor, Sarcoma, |
| | | | | | Skin tumor |
| 4-Acridinecarboxamide, | NSC 601316 | U.S. Pat. No. 05696131. | | | Brain tumor, Breast tumor, |
| N-[2- | | Journal Of Medicinal | | | Carcinoma, Colon tumor, |
| (dimethylamino)ethyl]- | | Chemistry 1987 30 664- | | | Lung tumor, Melanoma, |
| | | 669 | | | Ovary tumor, Sarcoma, |
| | | | | | Skin tumor |
| 9-chloro-10-hydroxy | SKF-108025 | Acs 1994 207th San | | | Carcinoma |
| camptothecin | | Diego MEDI 74 | | | |
| | CZ-105, | Proceedings Of The | | | Neoplasia |
| | CZ-107 | American Association Of | | | |
| | | Cancer Research 1997 | | | |
| | | 38 88 17 | | | |
| | JSKIV-47 | U.S. Pat. No. 05767142. | | | Neoplasia |
| | | WO 96/36612 | | | |
| | SKF-107874 | Acs Meeting 1994 207th | | | Carcinoma |
| | | San Diego MEDI 74 | | | |
| | Intoplicine | EP 402232 | | | Solid tumor |
| | CKD-602 | WO 96/21666. | | | Neoplasia |
| | Exetacan | EP 737686 | | | Leukemia, Myeloid |
| | mesylate; | | | | leukemia, Neoplasm, Non- |
| | exatecan | | | | small-cell lung cancer, |
| | | | | | Pancreas tumor |
| | IST-622 | EP 159708 | | | Neoplasia |
| | NB-506 | WO 93/11145 | | | Neoplasia |
| | Pyrazolo- | EP 138302 | | | Neoplasia |
| | acridine, | | | | |
| | Parke- | | | | |
| | Davis | | | | |
| | XR-5000 | U.S. Pat. No. 5696131 | | | Brain tumor, Breast tumor, |
| | | | | | Carcinoma, Colon tumor, |
| | | | | | Lung tumor, Melanoma, |
| | | | | | Ovary tumor, Sarcoma, |
| | | | | | Skin tumor |
| | DB-67 | WO 99/09996 | | | Neoplasia |
| | DRF-1042 | WO 97/46563 | | | Neoplasia |
| | F-11782 | WO 96/12727 | | | Neoplasia |
| | XR-5944 | WO 98/17650 | | | Neoplasia |
| | BN-80915 | WO 99/11646. | | | Neoplasia |

Other DNA topoisomerase I inhibiting agents of interest that can be used in the methods, combinations and compositions of the present invention include the compounds described in the patents provided in Table 4, below. The therapeutic compounds of Table 4 can also be used in the methods, combinations and compositions of the present invention in a variety of forms, including acid form, salt form, racemates, enantiomers, zwitterions, and tautomers.

TABLE 4
Additional DNA topoisomerase I inhibitors

| Company | Reference | Oncology Indication |
|---|---|---|
| Abbott Laboratories | WO 97/15676 | Neoplasm |
| Arch Development Corp | WO 96/01127 | Neoplasm |
| Banyu Pharmaceutical Co. | EP 388956 | Neoplasm |
| Ltd.. | | |
| Bayer AG | WO 98/14459 | Neoplasm |
| Bayer AG | WO 98/14468 | Neoplasm, Lung tumor |
| Bayer AG | WO 98/15573 | Neoplasm |
| Bayer AG | WO 98/51703 | Neoplasm |
| BioNumerik | US 5597829 | Neoplasm |
| Pharmaceuticals Inc. | | |
| BioNumerik | WO 95/17187 | Neoplasm |
| Pharmaceuticals Inc. | | |
| BioNumerik | WO 95/29677 | Neoplasm |
| Pharmaceuticals Inc | | |
| BioNumerik | WO 98/04557 | Leukemia, Breast tumor, |
| Pharmaceuticals Inc. | | Colon tumor, Melanoma, |
| | | Lung tumor, Non-Hodgkin |
| | | lymphoma, Ovary tumor |
| BioNumerik | WO 98/35940 | Neoplasm, Leukemia |
| Pharmaceuticals Inc. | | |
| BioNumerik | WO 95/28404 | Neoplasm |
| Pharmaceuticals Inc. | | |
| Bristol-Myers Co. | BE-900735 | Carcinoma |
| Bristol-Myers Squibb Co. | WO 98/07433 | Neoplasm |
| Chong Kun Dang Corp. | WO 96/21666 | Neoplasm, Leukemia |
| Chong Kun Dang Corp. | WO 99/02530 | Neoplasm |
| Daiichi Seiyaku Co Ltd. | JP-9020778 | Carcinoma |
| Dana-Farber Cancer | WO 97/07797 | Prostate disease, Ovary |
| Institute Inc. | | tumor, Breast tumor |
| Dr Reddys Research | WO 97/46562 | Leukemia, HIV infection |
| Foundation | | |
| FermaLogic Inc. | US 5554519 | Colon tumor |
| George Washington | WO 99/65493 | Diarrhea, Breast tumor, |
| University | | Ovary tumor, Colon tumor, |
| | | Melanoma, Lung tumor, |
| | | Thyroid tumor, Lymphoma, |
| | | Leukemia |
| Dr Reddys Research | WO 97/46564 | Leukemia, Neoplasm |
| Foundation | | |
| Glaxo Inc. | EP 540099 | Neoplasm |
| Glaxo Inc. | GB-2280674 | Carcinoma, Neoplasm |
| Glaxo Inc. | WO 94/25466 | Neoplasm |
| | WO 96/11005 | Neoplasm |
| Istituto Nazionale studio e | WO 97/31003 | Neoplasm |
| cura dei tumori | | |
| Johns Hopkins University | WO 96/08249 | Trypanosomiasis, |
| | | Leishmania infection |
| Kaken Pharmaceutical Co. | JP-11246469 | Neoplasm |
| Ltd. | | |
| Kyorin Pharmaceutical Co. | WO 96/41806 | Neoplasm |
| Ltd. | | |
| Matrix Pharmaceutical Inc. | WO 98/36776 | Neoplasm |
| Ohio State University | US 5552156 | Neoplasm |
| Pharmacia & Upjohn SpA | WO 95/22549 | Neoplasm |
| Pharmacia & Upjohn SpA | WO 95/32207 | Leukemia, Colon tumor |
| Pharmacia & Upjohn SpA | WO 97/25332 | Neoplasm |
| Pharmacia & Upjohn SpA | WO 98/35969 | Carcinoma, Leukemia |
| Pharmacia & Upjohn SpA | WO 99/17804 | Neoplasm |
| Pharmacia & Upjohn SpA | WO 95/04736 | Neoplasm, Leukemia |
| Pharmacia & Upjohn SpA | WO 99/05103 | Neoplasm |
| Pharmacia & Upjohn SpA | WO 99/17805 | Neoplasm |
| Pharmacia Inc. | WO 96/11669 | Neoplasm, Leukemia |
| Research Triangle Institute | WO 96/02546 | Neoplasm |
| Research Triangle Institute | WO 91/04260 | Neoplasm |
| Research Triangle Institute | WO 91/05556 | Colorectal tumor, |
| | | Leukemia, Colon tumor |
| Research Triangle Institute | WO 96/09049 | Plasmodium infection |
| Research Triangle Institute | WO 97/19085 | Neoplasm, Leukemia, |
| | | Colon tumor |
| Rockefeller University | WO 97/44492 | Neoplasm |
| Rutgers University | US 5767142 | Neoplasm, Burkitts |
| | | lymphoma, Myeloprolif- |
| | | erative disorder, Breast |
| | | tumor |
| Rutgers University | WO 98/31673 | Neoplasm, Fungal infection |
| Rutgers University | WO 99/31067 | Malignant neoplastic |
| | | disease, Solid tumor, |
| | | Leukemia |
| Rutgers University | WO 99/41241 | Malignant neoplastic |
| | | disease, Solid tumor, |
| | | Leukemia, Lymphoma, |
| | | Fungal infection |
| Rutgers University | WO 98/12181 | Leukemia, Melanoma, |
| | | Carcinoma |
| Rutgers University | WO 99/33824 | Solid tumor, Sarcoma, |
| | | Melanoma, Lymphoma |
| Sankyo Co Ltd. | JP-7316091 | Neoplasm |
| Shionogi & Co Ltd. | JP-7138165 | Carcinoma |
| SmithKline Beecham Corp. | EP 835938 | Staphylococcus infection |
| SmithKline Beecham Corp. | US 5633016 | Solid tumor |
| SmithKline Beecham Corp. | US 5674872 | Ovary tumor |
| SmithKline Beecham Corp. | WO 92/14469 | Neoplasm, Ovary tumor |
| SmithKline Beecham Corp. | WO 95/03803 | Viral infection |
| SmithKline Beecham Corp. | WO 96/38146 | Neoplasm |
| SmithKline Beecham Corp. | WO 96/38449 | Neoplasm |
| SmithKline Beecham Corp. | WO 92/05785 | Neoplasm |
| SmithKline Beecham Corp. | WO 92/14471 | Neoplasm |
| SmithKline Beecham Corp. | WO 92/14470 | Esophageal disease, |
| | | Neoplasm |
| SmithKline Beecham plc | WO 92/07856 | Viral infection |
| Societe de Conseils de | WO 98/28305 | Colon tumor, Lung tumor, |
| Recherches et d' | | Breast tumor, viral |
| Applications Scientifique | | infection, Parasitic |
| | | infection |
| Societe de Conseils de | WO 99/33829 | Colon tumor, Lung tumor, |
| Recherches et d' | | Leukemia, Leishmania |
| Applications Scientifique | | infection, Plasmodium |
| | | infection, Trypanosomiasis |
| Stehlin Foundation For | WO 97/28165 | Neoplasm, Carcinoma, |
| Cancer Research | | Breast tumor |
| Takeda Chemical Industries | EP 556585 | Neoplasm |
| Ltd. | | |
| Tanabe Seiyaku Co Ltd. | JP-11071280 | Neoplasm, Lung tumor |
| University of California | US 5698674 | Neoplasm, Viral infection |
| University of Michigan | WO 96/34003 | Breast tumor, Lung tumor, |
| | | Prostate tumor |
| University of New Jersey | WO 97/29106 | Neoplasm, Central nervous |
| | | system disease |
| University of New Jersey-- | WO 96/36612 | Burkitts lymphoma, |
| | | Leukemia, |
| | | Myeloproliferative disorder |
| University of Pittsburgh-- | WO 99/01456 | Malignant neoplastic |
| | | disease |
| Wisconsin Alumni | WO 96/33988 | Prostate tumor, Colon |
| Research Foundation | | tumor, Lung tumor, |
| | | Melanoma, Breast tumor, |
| | | HIV infection |
| Wisconsin Alumni | WO 97/31936 | Neoplasm |
| Research Foundation | | |
| Xenova Ltd. | WO 98/17649 | Neoplasm |
| Yale University | WO 98/40104 | Carcinoma |

Additional DNA topoisomerase I inhibiting agents of interest that can be used in the methods, combinations and compositions of the present invention are provided in Table 5, below. The therapeutic compounds of Table 5 can be used in the methods, combinations and compositions of the present invention in a variety of forms, including acid form, salt form, racemates, enantiomers, zwitterions, and tautomers.

TABLE 5
Additional DNA topoisomerase I inhibitors

| Compound Name | Company |
|---|---|
| BAY-38-3441 | Bayer AG |
| BNP-1350 | BioNumerik |
| GG-211 | Tigen |
| J-107088 | Merck & Co |
| kareniticin | BioNumerik Pharmaceuticals Inc |
| L9NC | MD Anderson Cancer Center |
| lurtotecan, Gilead | Gilead Sciences |
| MAG-CPT | Pharmacia |
| PEG-camptothecin, Enzon | Enzon |
| SN-22995 | University of Auckland |
| TRK-710 | Toray Industries Inc |
| NX-211 | Glaxo Wellcome plc |
| pyrazoloacridine, Wayne State | Non-industrial source |
| TAS-103 | Taiho |
| XR-5000 | Xenova |
| 9-aminocamptothecin | IDEC; Research Triangle Institute |
| rubitecan | SuperGen; Stehlin Foundation For |
| | Cancer Research |
| 10-hydroxycamptothecin derivatives, | Chiba University |
| Chiba | |
| AG-555 | Hebrew University of Jerusalem |
| anhydrous delivery system, Matrix | Matrix Pharmaceutical Inc |
| ascididemin | INSERM |
| BM-2419-1 | Kaken Pharmaceutical Co Ltd. |
| camptothecin analogs, RTI/BMS | Research Triangle Institute |
| camptothecin-TCS, Inex | Inex Pharmaceuticals Corp |
| CT-17 | University of Kentucky |
| DMNQ derivatives, Chungnam | Chungnam University |
| University | |
| DRF-1644 | Dr Reddys Research Foundation |
| dual topoisomerase I/II-directed | University of Auckland |
| anticancer drugs, University of | |
| Auckland | |
| HAR-7 | Harrier Inc |
| J-109404 | Banyu Pharmaceutical Co Ltd. |
| julibrosides | Taisho Pharmaceutical Co Ltd. |
| MPI-5019 | Matrix Pharmaceutical Inc |
| NSC-314622 | National Cancer Institute |
| NU/ICRF-505 | Imperial Cancer Research |
| | Technology Ltd. |
| NU-UB-150 | Napier University of Edinburgh |
| topoisomerase I inhibitors, Glaxo | Glaxo Wellcome plc |
| topoisomerase I inhibitors, | MediChem. Research Inc |
| MediChem/Mayo | |
| topoisomerase I inhibitors, Purdue | Purdue University |
| University/NCI | |
| topoisomerase I inhibitors, SMT | Morphochem Inc |
| topoisomerase inhibitor, Daiichi | Daiichi Seiyaku Co Ltd. |
| UCE-1022 | Kyowa Hakko Kogyo Co Ltd. |
| camptothecin, Aphios | Aphios |
| F-12167 | Pierre Fabre |
| ST-1481 | Sigma-Tau |
| topoisomerase inhibitors, BTG | BTG |
| XR-11576 | Xenova |
| gemifloxacin mesylate | LG Chemical |
| BN-80245 | Institut Henri Beaufour |

Other DNA topoisomerase I inhibiting agents of interest that can be used include the compounds described in the patents provided in Table 6, below. The therapeutic compounds of Table 6 can also be used in the methods, combinations and compositions of the present invention in a variety of forms, including acid form, salt form, racemates, enantiomers, zwitterions, and tautomers.

TABLE 2
New topoisomerase I inhibitors

| No. | Compound Name | Trade Name(s) | Drug Class | Dose | Manufacturer | Reference |
|---|---|---|---|---|---|---|
| A2 | Camptothecin in combination with poly- | | DNA Topoisomerase I | | | Singer, J. W. et al., Conjugation of |
| | (l-glutamic acid) | | Inhibitor | | | camptothecins to poly-(l-glutamic |
| | | | | | | acid), Annals of the New York |
| | | | | | | Academy of Sciences 922:136-150 |
| | | | | | | (2000). |
| A2 | Camptothecin in combination with | | NU1025: poly(ADP- | | | Br J Cancer. 2001 Jan |
| | NU1025 | | ribose) polymerase | | | 5;84(1):106-12. |
| | | | (PARP) inhibitor | | | |
| A3 | XR-11612 | | Dual inhibitor of | | Xenova Group plc | http://www.xenova.co.uk/PressRe |
| | | | topoisomerase I and II | | | leases/pr_20011031_01.html |
| A4 | DX-8951f | Exatecan | Camptothecin | 1 mg/m² by | Daiich | Giles, F. J., et al., Phase I and |
| | | mesylate | derivative; | 30 minute | Pharmaceutical Co. | pharmacokinetic study of DX- |
| | | | Topoisomerase I | IV infusion | | 8951f(exatecan mesylate), a |
| | | | inhibitor | on days 1, | | hexacyclic camptothecin, on a |
| | | | | 8 and 15 of | | daily-times-five schedule in |
| | | | | a 28 day | | patients with advanced leukemia. |
| | | | | cycle | | Clin Cancer Res. 8:2134-41 |
| | | | | | | (2002). |
| A5 | Anthracycline Aclacinomycin A | | DNA Topoisomerase I | | | John L. Nitiss, PhD; |
| | | | Inhibitor | | | http://www.stjude.org/molecular- |
| | | | | | | pharmacology/0,2536,428_2289_{—} |
| | | | | | | 3501,00.html |
| A6 | 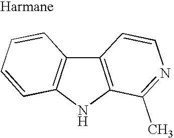 | | Peganum harmala L. (Zygophyllaceae)seeds extract | 700 mg/ml | | Sobhan, A. M., et al., An in vitro evaluation of human DNA topoisomerase I inhibition by Peganum Harmala L. seeds extract and its b-carboline alkaloids, J Pharm. Pharmaceut. Sci., 5:19-23(2002). |
| A7 | 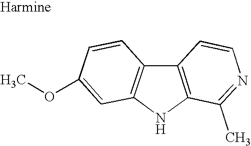 | | Peganum Harmala L. (Zygophyllaceae) seeds extract | 700 mg/ml | | Sobhan, A. M. et al., An in vitro evaluation of human DNA topoisomerase I inhibition by Peganum Harmala L. seeds extract and its b-carboline alkaloids, J Pharm. Pharmaceut. Sci., 5:19-23 (2002). |
| A8 | 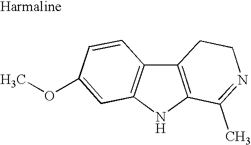 | | Peganum harmala L. (Zygophyllaceae) seeds extract | 700 mg/ml | | Sobban, A. M., et at., An in vitro evaluation of human DNA topoisomerase I inhibition by Peganum Harmala L. seeds extract and its b-carboline alkaloids, J Pharm. Pharmaceut. Sci., 5:19-23 (2002). |
| A9 | Bulgarein | | Fungal metabolite; | 0.025-5 | | Fujii, N., et at., Induction of |
| | | | topoisomerase I | microM | | mammalian DNA topoisomerase |
| | | | inhibitor | | | I-mediated DNA cleavage and |
| | | | | | | DNA winding by bulgarein, I. |
| | | | | | | Biol. Chem., 268:13160-5 (1993). |
| A10 | 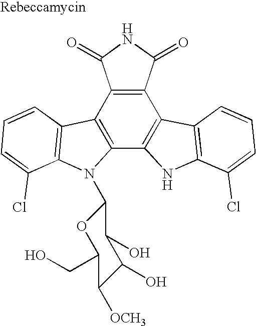 | | Indolocarbazole ferivative; topoisomerase I inhibitor | | | Labourier, E., et at., Poisoning of topoisomerase I by an antitumor indolocarbazole drug: stabilization of topoisomerase I- DNA covalent complexes and specific inhibition of the protein kinase activity, Cancer Research 59:52-5 (1999). |
| A11 | 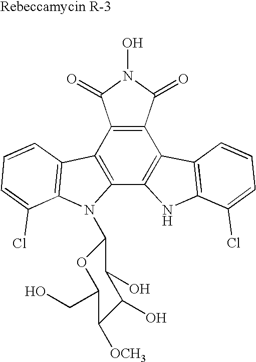 | | Indolocarbazole derivative; topoisomerase I inhibitor | | | Labourier, E., et al., Poisoning of topoisomerase I by an antitumor indolocarbazole drug: stabilization of topoisomerase I- DNA covalent complexes and specific inhibition of the protein kinase activity, Cancer Research 50:52-5 (1999). |
| A12 | 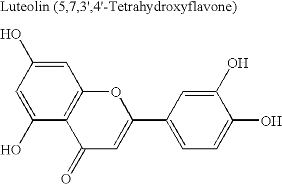 | | Flavonoid; topoisomerase I inhibitor | 40 μM, with an IC₅₀ of 5 μM | | Chowdhury, A. R., et al., Luteolin, an emerging anti-cancer flavonoid, poisons eukaryotic DNA topoisomerase I, Biochem. J. 366:653-661 (2002). |
| A13 | Diospyrin | | Bisnaphthoquinone | | | Ray, S., et al., Diospyrin, a |
| | | | | | | bisnaphthoquinone: a novel |
| | | | | | | inhibitor of type I DNA |
| | | | | | | topoisomerase of leishmania |
| | | | | | | donovani, Molecular |
| | | | | | | Pharmacology, 54: 994-9 (1998). |
| A14 | 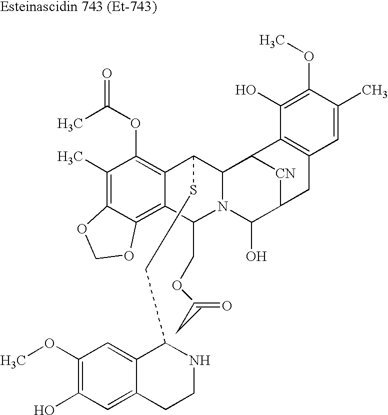 | | Ecteinascidia turbinateantitumor agent | | | Yuji T., et al., Poisoning human DNA topoisomerase I by ecteinascidin 743, an anticancer drug that selectively alkylates DNA in the minor groove, Proc. Natl. A cad. Sci. USA. 96:7196-7201 (1999). |
| A15 | Ho-33342 | | DNA minor groove | | | Chen, A. Y., et al., A new |
| | | | binding ligand; DNA | | | mammalian DNA topoisomerase I |
| | | | Topoisomerase I | | | poison Hoechst 33342: |
| | | | inhibitor | | | cytotoxicity and drug resistance in |
| | | | | | | human cell cultures, Cancer |
| | | | | | | Research, 53:1332-7, (1993). |
| A16 | Ho-33258 | | DNA minor groove | | | Chen, A. Y., et al., A new |
| | | | binding ligand; DNA | | | mammalian DNA topoisomerase I |
| | | | Topoisomerase I | | | poison Hoechst 33342: |
| | | | inhibitor | | | cytotoxicity and drug resistance in |
| | | | | | | human cell cultures, Cancer |
| | | | | | | Research, 53:1332-7, (1993). |
| A17 | 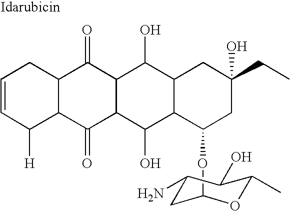 | | Disaccharide analog; Topoisomerase I and II inhibitor | | | Guano, F., et al., Topoisomerase Poisoning Activity of Novel Disaccharide Anthracyclines, Molecular Pharmacology, Vol. 56, Issue 1, 77-84, July 1999 |
| A18 | SN-38 in combination with 5-FU in | | Topoisomerase I | | | Bernacki, R. J., et al., In vitro |
| | sequential drug administration, SN-38 | | Inhibitor | | | antitumor activity of 9-nitro- |
| | first | | | | | camptothecin as a single agent |
| | | | | | | and in combination with other |
| | | | | | | antitumor drugs, Ann. N. Y. Acad. |
| | | | | | | Sci. 922:293-7 (2000). |
| A19 | 9-NC in combination with 5-FU | | Topoisomerase I | | | Bernacki, R. J., et al., In vitro |
| | | | Inhibitor | | | antitumor activity of 9-nitro- |
| | | | | | | camptothecin as a single agent |
| | | | | | | and in combination with other |
| | | | | | | antitumor drugs, Ann. N. Y. Acad. |
| | | | | | | Sci. 922:293-7 (2000). |
| A20 | BN-80927 | | Topoisomerase I and II | | | Bernacki, R. J., et al., In vitro |
| | | | Inhibitor | | | antitumor activity of 9-nitro- |
| | | | | | | camptothecin as a single agent |
| | | | | | | and in combination with other |
| | | | | | | antitumor drugs, Ann. N. Y. Acad. |
| | | | | | | Sci. 922:293-7 (2000). |
| A21 | 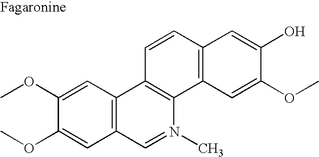 | | Topoisomerase I poison | | | The Pharmaceutical Journal, 267(7169):510-525 (2001). |
| A22 | 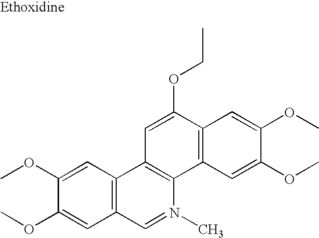 | | Topoisomerase I suppressor | | | The Pharmaceutical Journal, 267(7169):510-525 (2001). |
| A23 | 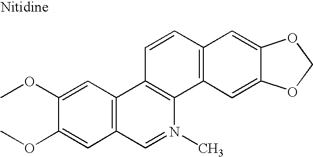 | | Topoisomerase I and II inhibitor | | | Fleury, F., et al., Molecular determinants of site-specific inhibition of human DNA topoisomerase I by fagaronine and ethoxidine. Relation to DNA binding, J Bid Chem, 275 3501-9 (2000). |
| A24 | MJ-III-65 | | Noncamptothecin | | | Antony, Smitha, Molecular |
| | | | Topoisomerase I Poison | | | Biology, |
| | | | | | | http://festival02.nih.gov/sessions/ |
| | | | | | | CategoryMOL.html |
| A25 | S2 | | G. fascicularis; | | | Frank Marini, An Advanced |
| | | | topoisomerase I | | | Aquarist's Short Take: Bioactive |
| | | | inhibitor | | | Agents in the World's Reefs, |
| | | | | | | http://www.advancedaquarist.com/ |
| | | | | | | issues/june2002/short.htm. |
| A26 | J-107088 [6-N-(1-hydroxymethyla-2- | | Topoisomerase I | 54 mg/kg | | Cavazos C. M., et al., Therapeutic |
| | hydroxyl) ethylamino-12,13-dihydro- | | inhibitor | per day in | | activity of the topoisomerase I |
| | 13-(beta-D-glucopyranosyl)-5H- | | | 10% | | inhibitor J-107088 [6-N-(1- |
| | indolo[2,3-a]-pyrrolo[3,4-c]-carbazole- | | | dimethyl | | hydroxymethyla-2-hydroxyl) |
| | 5,7(6H)-dione]] | | | sulfoxide in | | ethylamino-12,13-dihydro-13- |
| | | | | 0.9% saline | | (beta-D-glucopyranosyl)-5H- |
| | | | | in mice. | | indolo[2,3-a]-pyrrolo[3,4-c]- |
| | | | | | | carbazole-5,7(6H)-dione]] against |
| | | | | | | pediatric and adult central nervous |
| | | | | | | system tumor xenografts, Cancer |
| | | | | | | Chemother Pharmacol., 48:250-4 |
| | | | | | | (2001). |
| A27 | Kareinitecin (BNP-1100) | | lipophilic, silylated | 1 mg/m² | | Daud, A., et al., Phase II trial of |
| | | | camptothecin | I.V. daily × | | karenitecin, a novel |
| | | | topoisoinerase I | 5 days, | | topoisomerase I inhibitor in |
| | | | inhibitor | repeated | | metastatic melanoma: Clinical and |
| | | | | every 21 | | translational study, American |
| | | | | days. | | Society of Clinical Oncology, |
| | | | | | | Abstract No. 2871 (2003). |
| A28 | BNP-1100 in combination with ZD- | | Thymidylate synthase | | | Matsui, S., et al., Characterization |
| | 1694 | | inhibitor in | | | of a synergistic interaction |
| | | | combination | | | between a thymidylate synthase |
| | | | topoisomerase I | | | inhibitor, ZD1694, and a novel |
| | | | inhibitor. | | | lipophilic topoisomerase I |
| | | | | | | inhibitor karenitecin, BNP1100: |
| | | | | | | mechanisms and clinical |
| | | | | | | implications, Eur. J. Cancer, |
| | | | | | | 35:984-93 (1999). |
| A29 | 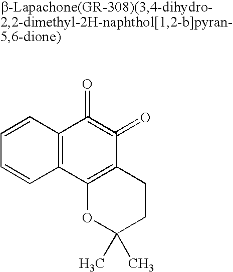 | | Topoisomerase I inhibitor | | | Boothman, D. A., et al., Inhibition of potentially lethal DNA damage repair in human tumor cells by beta-lapachone, an activator of topoisomerase, I, Cancer Research, 49:605-612 (1989). |
| A30 | Intoplicine (RP-60475) 7H- | | Dual topoisomerase I | | | Poddevin, B., et al., Dual |
| | benzo[e]pyrido[4,3-b]indole derivative | | and II inhibitor | | | Topoisomerase I and II Inhibition |
| | | | | | | by Intoplicine (RP-60475), a New |
| | | | | | | Antitumor Agent in Early Clinical |
| | | | | | | Trials, Molecular Pharmacology, |
| | | | | | | 44:767-774 (1993). |
| A31 | TAN-1518A | | Non-cleavable complex | | | Nishizawa, M., et al., New |
| | | | stabilizing type of | | | Topoisomerase I Inhibitors, TAN- |
| | | | topoisomerase I | | | 1518A and Pta quiloside: Their |
| | | | inhibitors; inhibits | | | Mode of Action. |
| | | | CPT-resistant | | | http://www.acc.pref.aichi.jp/acc/ |
| | | | topoisomerase I | | | english/94_95/divisions/biochem/ |
| | | | | | | biochem.htm; Horiguchi et al., J. |
| | | | | | | Antibiotics 47: 545, 1994; |
| | | | | | | Horiguchi and Tanida, Biochem. |
| | | | | | | Pharmacol. 49: 1395, 1995 |
| A32 | Plaqualoside | | Non-cleavable complex | | | Nishizawa, M., et al., New |
| | | | stabilizing type of | | | Topoisomerase 1 Inhibitors, TAN- |
| | | | topoisomerase I | | | 1518A and Plaquiloside: Their |
| | | | inhibitors; inhibits | | | Mode of Action. |
| | | | CPT-resistant | | | http://www.acc.pref.aichi.jp/acc/ |
| | | | topoisomerase I | | | english/9495/divisions/biochem/ |
| | | | | | | biochem.htm |
| A33 | GI-147211 | | Semisynthetic | 0.3 to 1.5 | | Gerrits, C. J., et al., Phase I and |
| | | | camptothecin analogue; | mg m-2 | | pharmacological study of the new |
| | | | topoisomerase I | day-1; 30 | | topoisomerase I inhibitor |
| | | | inhibitor | min iv | | G114721 1, using a daily × 5 |
| | | | | infusion | | intravenous administration, Br J |
| | | | | daily for 5 | | Cancer, 73.744-50 (1996). |
| | | | | consecu- | | |
| | | | | tive days, | | |
| | | | | repeated | | |
| | | | | every 3 | | |
| | | | | weeks | | |
| A34 | Camptothecin in combination with 7- | | Topoisomerase I | | | U.S. Pat. No. 6,214,821 |
| | hydroxystaurosporine | | inhibitor | | | |
| A35 | Indenoisoquinolines | | Topoisomerase I | | | U.S. Pat. No. 6,509,344 |
| | | | inhibitors | | | |
| A36 | Heteroaromatic[a]phenazine | | Topoisomerase I/ | | | U.S. Pat. No. 6,552,021 |
| | carboxamide derivatives | | topoisomerase II | | | |
| | | | inhibitors | | | |
| A37 | Covalent conjugates of topoisomerase I | | Topoisomerase I/ | | | U.S. Pat. No. 6,420,377 |
| | and topoisomerase II inhibitors | | topoisomerase II | | | U.S. Pat. No. 6,207,673 |
| | | | inhibitors | | | |
| A38 | 7-substituted camptothecin derivatives | | Topoisomerase I | | | U.S. Pat. No. 6,306,868 |
| | | | inhibitors | | | U.S. Pat. No. 6,130,227 |
| A39 | Highly lipophilic camptothecin | | Topoisomerase I | | | U.S. Pat. No. 6,169,080 |
| | derivatives | | inhibitors | | | U.S. Pat. No. 6,057,303 |
| A40 | Hexacyclic camptothecin analogues | | Topoisomerase I | | | U.S. Pat. No. 5,990,120 |
| | | | inhibitor | | | |
| A41 | Trisbenzimidazoles | | Topoisomerase I | | | U.S. Pat. No. 5,948,797 |
| | | | inhibitors | | | U.S. Pat. No. 5,807,874 |
| A42 | Benzo[a]phenazine-11-carboxamide | | Topoisomerase I/ | | | U.S. Publication No. 20030139409 |
| | derivatives | | topoisomerase II | | | |
| | | | inhibitors | | | |
| A43 | XR 5000 | | Topoisomerase I/ | | | Dittrich, c., et al., Phase II study |
| | N-[2-(dimethylamino)ethyl]acridine-4- | | topoisomerase II | | | of XR 5000 (DACA), an inhibitor |
| | carboxamide | | inhibitor | | | of topoisomerase I and II, |
| | | | | | | administered as a 120-h infusion |
| | | | | | | in patients with non-small cell |
| | | | | | | lung cancer. Eur J Cancer. |
| | | | | | | 39:330-4 (2003). |
| A44 | Phenoxodiol | | Topoisomerase II | | | Constantinou, A. I. and Husband, |
| | 2H-1-benzopyran-7-0,1,3-(4- | | inhibitor | | | A., Phenoxodiol (2H-1- |
| | hydroxyphenyl) | | | | | benzopyran-7-0,1,3-(4- |
| | | | | | | hydroxyphenyl)), a novel |
| | | | | | | isoflavone derivative, inhibits |
| | | | | | | DNA topoisomerase II by |
| | | | | | | stabilizing the cleavable complex. |
| | | | | | | Anticancer Res. 22:2581-5 (2002). |
| A45 | AHMA | | Topoisomerase | | | Dong, X., Crystallization and |
| | 3-(9-acridinylamino)-5- | | inhibitor | | | preliminary X-ray analysis of |
| | (hydroxymethyl)aniline | | | | | anti-cancer agent 3-(9- |
| | | | | | | acridinylamino)-5- |
| | | | | | | (hydroxymethyl)aniline |
| | | | | | | complexed with the DNA |
| | | | | | | hexamer d(CGTACG)2. Biochim |
| | | | | | | Biophys Acta. 1625:27-9 (2003). |
| A46 | (5Z,9Z)-5,9-hexadecadienoic acid | | Topoisomerase I | | | Carballeira, N. M., et al., Total |
| | | | inhibitor | | | synthesis and biological |
| | | | | | | evaluation of (5Z,9Z)-5,9- |
| | | | | | | hexadecadienoic acid, an inhibitor |
| | | | | | | of human topoisomerase I. J Nat |
| | | | | | | Prod. 65: 1715-8 (2002). |
| A47 | RFS2000 | | Topoisomerase I | | | Raymond, E., et al., Muhicentre |
| | 9-nitro-camptothecin | | inhibitor | | | phase II and pharmacokinetic |
| | | | | | | study of RFS2000 (9-nitro- |
| | | | | | | camptothecin) administered orally |
| | | | | | | 5 days aweek in patients with |
| | | | | | | glioblastoma multiforme. Eur J |
| | | | | | | Cancer. 38:1348-50 (2002). |
| A48 | 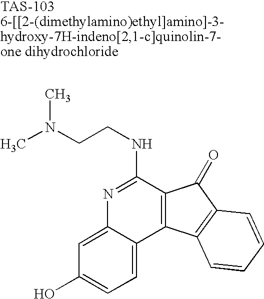 | | Topoisoinerase I/ topoisomerase II inhibitor | | | Mizutani, H., et al., Mechanism of apoptosis induced by a new topoisomerase inhibitor through the generation of hydrogen peroxide. J Bio Chem. 277:30684-9 (2002). |
| A49 | 7-ethyl-10-[4-(1-piperidyl)-1-piperidyl] | | Topoisomerase I | | | Kohara, H., et al., Synergistic |
| | carbonyloxy-camptothecin | | inhibitor | | | effects of topoisoinerase I |
| | | | | | | inhibitor, 7-ethyl-10- |
| | | | | | | hydroxycamptothecin, and |
| | | | | | | irradiation in a cisplatin-resistant |
| | | | | | | human small cell lung cancer cell |
| | | | | | | line. Clin Cancer Res. 8:287-92 |
| | | | | | | (2002). |
| A50 | 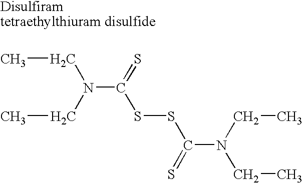 | Antabuse | Topoisomerase I/ topoisomerase II inhibitor | | Wyeth Ayerst | Yakisich, J. S., et at., Disulfiram is a potent in vitro inhibitor of DNA topoisomerases. Biochem Biophys Res Commun. 289:586-90 (2001). |
| A51 | Isoaurostatin | | Topoisomerase I | | | Suzuki, K., et at., Isoaurostatin, a |
| | 6,4′-dihydroxyisoaurone | | inhibitor | | | novel topoisomerase inhibitor |
| | | | | | | produced by Thermomonospora |
| | | | | | | alba. J Nat Prod. 64:204-7 (2001). |
| A52 | 6-[3-(2-hydroxyethyl)aminopropyl]-5,6- | | Topisomerase I | | | Cushman, M., et al., Synthesis of |
| | dihydro-2,3-dimethoxy-8,9- | | inhibitor | | | new indeno[1,2-c]isoquinolines: |
| | methylenedioxy-5,11-dioxo-11H- | | | | | cytotoxic non-camptothecin |
| | indeno[1,2-c]isoquinoline hydrochloride | | | | | topoisomerase I inhibitors. J Med |
| | | | | | | Chem. 43:3688-98 (2000). |
| A53 | BNP1350 | | semi-synthetic, highly | | | Van Hattum, A. H., et al., New |
| | 7-[(2-trimethylsilyl)ethyl]-20(S)- | | lipophilic, silicon- | | | highly lipophilic camptothecin |
| | camptothecin | | containing | | | BNP1350 is an effective drug in |
| | | | camptothecin; inhibitor | | | experimental human cancer. Int J |
| | | | of topoisomerase I | | | Cancer. 88:260-6 (2000). |
| A54 | Ring-substituted 11-oxo-11H- | | Topoisomerase I/ | | | Deady, L. W., et at., Ring- |
| | indeno[1,2-b]quinoline-6-carboxamides | | topoisomerase II | | | substituted 11-oxo-11H- |
| | | | inhibitor | | | indeno[1,2-b]quinoline-6- |
| | | | | | | carboxamides with similar |
| | | | | | | patterns of cytotoxicity to the dual |
| | | | | | | topo I/II inhibitor DACA. Bioorg |
| | | | | | | Med Chem. 7:2801-9 (1999). |

One of skill in the art will understand how to make the DNA topoisomerase I inhibitors described above by following the teachings of the corresponding references.

The Cox-2 inhibitors and DNA topoisomerase I inhibitors can be supplied in the form of a prodrug, an isomer, a tautomer, a racemic mixture, pharmaceutically-acceptable salt, or in any other chemical form or combination that, under physiological conditions, provides the Cox-2 inhibitor and DNA topoisomerase I inhibitor. The present invention includes all possible diastereomers as well as their racemic and resolved, enantiomerically pure forms and pharmaceutically acceptable salts thereof.

The Cox-2 inhibitors and DNA topoisomerase I inhibitors that are useful in the present invention can be of any purity or grade, as long as the preparation is of a quality suitable for pharmaceutical use. For example, the Cox-2 inhibitors and DNA topoisomerase I inhibitors can be provided in pure form, or it can be accompanied with impurities or commonly associated compounds that do not affect its physiological activity or safety. Each of the Cox-2 inhibitors and DNA topoisomerase I inhibitors of the present invention can be supplied as a pure compound, a pharmaceutically acceptable salt, or a prodrug, if desirable.

The Cox-2 inhibitors and DNA topoisomerase I inhibitors can also be supplied in the form of an isomer, a racemic mixture, or in any other chemical form or combination that, under physiological conditions, still provides for inhibition of the Cox-2 enzyme and inhibition of the DNA topoisomerase I enzyme.

In preferred embodiments, the present invention also encompasses a therapeutic composition for preventing or treating a neoplasia or neoplasia-related disorder in a subject that is in need of such prevention or treatment comprising a Cox-2 inhibitor and a DNA topoisomerase I inhibitor.

In other preferred embodiments, the present invention encompasses a pharmaceutical composition for preventing or treating a neoplasia or neoplasia-related disorder in a subject that is in need of such prevention or treatment comprising a Cox-2 inhibitor, a DNA topoisomerase I inhibitor, and a pharmaceutically acceptable carrier.

The pharmaceutical compositions of the present invention comprise a Cox-2 inhibitor and a DNA topoisomerase I inhibitor as an active ingredient or a pharmaceutically acceptable salt, thereof, and also contain a pharmaceutically acceptable carrier and optionally other therapeutic ingredients. When the Cox-2 inhibitor and a DNA topoisomerase I inhibitor are supplied along with a pharmaceutically acceptable carrier, a pharmaceutical composition is formed. A pharmaceutical composition of the present invention is directed to a composition suitable for the prevention, treatment, or amelioration of a neoplasia or neoplasia-related disorder. The pharmaceutical composition comprises a pharmaceutically acceptable carrier, a Cox-2 inhibitor, and a DNA topoisomerase I inhibitor.

The term “pharmaceutically acceptable” is used herein to mean that the modified noun is appropriate for use in a pharmaceutical product.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. In one embodiment the Cox-2 inhibitor and the DNA topoisomerase I inhibitor are administered to a subject together in one pharmaceutical carrier. In another embodiment, the Cox-2 inhibitor and the DNA topoisomerase I inhibitor are administered separately.

The pharmaceutically acceptable carrier can also be selected on the basis of the desired route of administration of the compound. For example, in a preferred embodiment the carrier is suitable for oral administration. In a more preferred embodiment, the composition includes a carrier or additional agent that is suitable for promoting delivery of the compound to the brain. Carriers that can promote delivery of the compound to the brain can include any carrier that promotes translocation across the blood-brain barrier and any carrier that promotes uptake of the compound by neural cells. Examples of such carriers include those disclosed in U.S. Pat. No. 5,604,198 (issued to Poduslo, et al.), U.S. Pat. No. 5,827,819 (issued to Yatvin, et al.), U.S. Pat. No. 5,919,815 (issued to Bradley, et al.), U.S. Pat. No. 5,955,459 (issued to Bradley, et al.), and U.S. Pat. No. 5,977,174 (issued to Bradley, et al.).

The terms “pharmaceutically acceptable salts” refer to salts prepared from pharmaceutically acceptable non-toxic bases including inorganic bases and organic bases. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, hydrochloric, trifluoroacetic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences.

Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

All of the above salts and ions can be prepared by those skilled in the art by conventional means from the corresponding compound of the present invention.

In the present invention, a Cox-2 inhibitor and a DNA topoisomerase I inhibitor are administered to a subject according to standard routes of drug delivery that are well known to one of ordinary skill in the art. The particular route and dosage of the Cox-2 inhibitor and the DNA topoisomerase I inhibitor depend upon the needs of the subject being treated, the type of treatment or prevention, the efficacy of the compound and the degree of disease severity in the subject.

The pharmaceutical compositions may be administered enterally and parenterally. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Enteral administration includes solution, tablets, sustained release capsules, enteric-coated capsules, and syrups. When administered, the pharmaceutical composition may be at or near body temperature.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the Cox-2 inhibitor and/or DNA topoisomerase I inhibitor may be formulated with sweetening agents, for example glycerol, sorbitol, or sucrose. Such formulations may also contain a demulcent, a preservative and flavoring and coloring agents.

The subject Cox-2 inhibitor and/or DNA topoisomerase I inhibitor and compositions comprising the same can also be administered parenterally, either subcutaneously, or intravenously, or intramuscularly, or intrasternally, or by infusion techniques, in the form of sterile injectable aqueous or olagenous suspensions. Parenteral administration includes subcutaneous, intramuscular, intradermal, intramammary, intravenous, and other administrative methods known in the art.

Such suspensions may be formulated according to the known art using those suitable dispersing of wetting agents and suspending agents, which have been mentioned above or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Administration of either one or both of the Cox-2 inhibitor and DNA topoisomerase I inhibitor can also be by inhalation, in the form of aerosols or solutions for nebulizers. Therefore, in one embodiment, the Cox-2 inhibitor and/or the DNA topoisomerase I inhibitor is administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Aerosols of liquid particles comprising the active materials may be produced by any suitable means, such as inhalatory delivery systems. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration.

One type of solid particulate aerosol generator is an insufflator. Suitable formulations for administration by insufflation include finely comminuted powders which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually-operated pump. The powder employed in the insufflator consists either solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the Cox-2 inhibitor and the DNA topoisomerase I inhibitor in a liquified propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

A third type of aerosol generator is a electrohydrodynamic (EHD) aerosol generating device, which has the advantage of being adjustable to create substantially monomodal aerosols having particles more uniform in size than aerosols generated by other devices or methods. Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric force exerted on the surface of the droplet overcomes the surface tension of the liquid, thereby generating a stream of liquid that disperses into a many smaller droplets of roughly the same size. These smaller droplets form a mist which constitutes the aerosol cloud that the user ultimately inhales.

Administration of the compositions of the present invention can also be rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Also encompassed by the present invention is buccal and sub-lingual administration, which includes lozenges or a chewable gum comprising the compounds set forth herein. The compounds can be deposited in a flavored base, usually sucrose, and acacia or tragacanth, and pastilles comprising the compounds in an inert base such as gelatin and glycerin or sucrose and acacia.

The prevent invention further encompasses intranasal administration comprising the compounds set forth herein. Intranasal dosage forms include, but are not limited to, aerosols, drops, gels, powders, and mixtures thereof.

Other methods for administration of the Cox-2 inhibitor compound and the DNA topoisomerase I inhibitor include dermal patches that release the medicaments directly into a subject's skin.

Topical delivery systems are also encompassed by the present invention and include ointments, powders, sprays, creams, jellies, collyriums, solutions or suspensions.

The compositions of the present invention can optionally be supplemented with additional agents such as, for example, viscosity enhancers, preservatives, surfactants and penetration enhancers.

Viscosity is an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a Cox-2 inhibitor topical composition or a Cox-2 inhibitor and DNA topoisomerase I inhibitor topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Pharmaceutically acceptable excipients and carriers encompass all the foregoing and the like. The above considerations concerning effective formulations and administration procedures are well known in the art and are described in standard textbooks. See e.g. Gennaro, A. R., Remington: The Science and Practice of Pharmacy, 20th Edition, (Lippincott, Williams and Wilkins), 2000; Hoover, John E., Remington's Pharmaceutical Sciences, Mack Publishing Co., Easton Pa., 1975; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

For purposes of the present invention, it is further preferred that the amount of a Cox-2 inhibitor and the amount of a DNA topoisomerase I inhibitor administered to the subject together comprise an effective amount of the combination of the two treatment agents. More preferred is that the amount of the co-therapy with the Cox-2 inhibitor and DNA topoisomerase I inhibitor comprises a therapeutically effective amount of the co-therapy.

Thus, the present invention encompasses a method of preventing and treating a neoplasia or neoplasia-related disorder in a subject in need of such prevention or treatment, the method comprising administering an amount of a Cox-2 inhibitor and an amount of a DNA topoisomerase I inhibitor wherein the amount of the Cox-2 inhibitor and the amount of the DNA topoisomerase I inhibitor together comprise a therapeutically effective amount.

In other embodiments, the pharmaceutical composition comprises a first amount of a Cox-2 inhibitor, or a pharmaceutically acceptable salt, ester or prodrug thereof; a second amount of a DNA topoisomerase I inhibitor, or a pharmaceutically acceptable salt, ester or prodrug thereof; and a pharmaceutically acceptable carrier; wherein the first and second amounts in combination comprise a therapeutically effective amount of said pharmaceutical composition.

As used herein, the term “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances. The dose or effective amount to be administered to a subject and the frequency of administration to the subject can be readily determined by one of ordinary skill in the art by the use of known techniques and by observing results obtained under analogous circumstances.

The term “pharmacologically effective amount” means that amount of a drug or pharmaceutical agent that will elicit the biological or medical response of a tissue, system, animal or human that is being sought by a researcher or clinician. This amount can be a therapeutically effective amount.

As used herein, the terms “therapeutically effective” or “therapeutically effective amount”, as used interchangeably herein, are intended to qualify the amount of an agent for use in therapy that will achieve the goal of preventing, ameliorating, or improving the severity of, the disorder being treated, while avoiding adverse side effects typically associated with alternative therapies. A symptom of a neoplasia or neoplasia-related disorder is considered ameliorated or improved if any benefit is achieved, no matter how slight.

It will be appreciated that the amount of the Cox-2 inhibitor and the DNA topoisomerase I inhibitor required for use in the treatment or prevention of neoplasia and neoplasia-related disorders will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

The appropriate dosage level of a Cox-2 inhibitor will generally be from about 0.01 mg per kg to about 140 mg per kg subject body weight per day, which may be administered in single or multiple doses. Preferably, the dosage level will be about 0.1 mg/kg to about 25 mg/kg per day; more preferably about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, preferably once or twice per day.

The amount of the Cox-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

The dosage level of the DNA topoisomerase I inhibitor will necessarily depend on the particular agent that is used. However, in general, the DNA topoisomerase I inhibitor is preferably present in the composition in an amount having an upper limit of about 1000 mg per kg subject body weight per day, preferably about 100 mg per kg, even more preferably about 10 mg per kg, and yet more preferably about 5 mg per kg, and preferably in an amount having a lower limit of about 0.001 mg per kg subject body weight per day, more preferably about 0.01 mg per kg, and yet more preferably about 1 mg per kg. The DNA topoisomerase I inhibitor may be administered to the subject on a regimen of 1 to 4 times per day.

A combination therapy comprising a DNA topoisomerase I inhibitor that is intended for the oral administration of humans may contain from about 10 micrograms to about 10 grams of the DNA topoisomerase I inhibitor, optionally compounded with an appropriate and convenient amount of carrier material, which may vary from about 5 to about 95 percent of the total composition. In preferred embodiments, the DNA topoisomerase I inhibitor is dosed in an amount having an upper limit of about 1 gram, more preferably about 500 mg, and yet more preferably about 100 mg, and preferably in an amount having a lower limit of about 0.01 mg, more preferably about 0.1 mg, and yet more preferably about 1 mg.

The exact dosage and regimen for administering a Cox-2 inhibitor alone or in combination with a DNA topoisomerase I inhibitor will necessarily depend upon the potency and duration of action of the compounds used, the nature and severity of the illness to be treated, as well as the sex, age, weight, general health and individual responsiveness of the patient to be treated, and other relevant circumstances. Those skilled in the art will appreciate that dosages may also be determined with guidance from Goodman & Goldman's The Pharmacological Basis of Therapeutics, Ninth Edition (1996), Appendix II, pp. 1707-1711.

Dosages for the combination therapy provided herein may be determined and adjusted based on the efficacy demonstrated in reducing or preventing the symptoms of a neoplasia and neoplasia-related disorder. In addition, one of ordinary skill in the art will know how to measure and quantify the presence or absence of neoplasia and neoplasia-related disorder symptoms.

The effectiveness of a particular dosage of a Cox-2 inhibitor in combination with a DNA topoisomerase I inhibitor is determined by staging the disorder at multiple points during a subject's treatment. For example, once a histologic diagnosis is made, staging (i.e., determination of the extent of disease) helps determine treatment decisions and prognosis. Clinical staging uses data from the patient's history, physical examination, and noninvasive studies. Pathologic staging requires tissue specimens.

Pathological staging is performed by obtaining a biopsy of the neoplasm or tumor. A biopsy is performed by obtaining a tissue specimen of the tumor and examining the cells microscopically. A bone marrow biopsy is especially useful in determining metastases from malignant lymphoma and small cell lung cancer. Marrow biopsy will be positive in 50 to 70% of patients with malignant lymphoma (low and intermediate grade) and in 15 to 18% of patients with small cell lung cancer at diagnosis. See The Merck Manual of Diagnosis & Therapy, Beers & Brakow, 17th edition, Published by Merck Research Labs, Sec. 11, Chapter 84, Hematology and Oncology, Overview of Cancer (1999).

Determination of serum chemistries and enzyme levels may also help staging. Elevation of liver enzymes (alkaline phosphatase, LDH, and ALT) suggests the presence of liver metastases. Elevated alkaline phosphatase and serum Ca²⁺ may be the first evidence of bone metastases. Elevated acid phosphatase (tartrate inhibited) suggests extracapsular extension of prostate cancer. Fasting hypoglycemia may indicate an insulinoma, hepatocellular carcinoma, or retroperitoneal sarcoma. Elevated BUN or creatinine levels may indicate an obstructive uropathy secondary to a pelvic mass, intrarenal obstruction from tubular precipitation of myeloma protein, or uric acid nephropathy from lymphoma or other cancers. Elevated uric acid levels often occur in myeloproliferative and lymphoproliferative disorders. α-Fetoprotein may be elevated in hepatocellular carcinoma and testicular carcinomas, carcinoembryonic antigen-S in colon cancer, human chorionic gonadotropin in choriocarcinoma and testicular carcinoma, serum immunoglobulins in multiple myeloma, and DNA probes (bcr probe to identify the chromosome 22 change) in CML.

Tumors may synthesize proteins that produce no clinical symptoms, e.g., human chorionic gonadotropin, ca-fetoprotein, carcinoembryonic antigen, CA 125, and CA 153. These protein products may be used as tumor markers in the serial evaluation of patients for determining disease recurrence or response to therapy. Thus, monitoring a subject for these tumor markers is indicative of the progress of a neoplasia disorder. Such monitoring is also indicative of how well the methods and compositions of the present invention are treating or preventing a neoplasia disorder. Likewise, tumor marker monitoring is effective to determine the appropriate dosages of the compositions of the present invention for treating neoplasia.

Other techniques include mediastinoscopy, which is especially valuable in the staging of non-small cell lung cancer. If mediastinoscopy shows mediastinal lymph node involvement, then the subject would not usually benefit from a thoracotomy and lung resection. Imaging studies, especially CT and MRI, can detect metastases to brain, lung, spinal cord, or abdominal viscera, including the adrenal glands, retroperitoneal lymph nodes, liver, and spleen. MRI (with gadolinium) is the procedure of choice for recognition and evaluation of brain tumors.

Ultrasonography can be used to study orbital, thyroid, cardiac, pericardial, hepatic, pancreatic, renal, and retroperitoneal areas. It may guide percutaneous biopsies and differentiate renal cell carcinoma from a benign renal cyst. Lymphangiography reveals enlarged pelvic and low lumbar lymph nodes and is useful in the clinical staging of patients with Hodgkin's disease, but it has generally been replaced by CT.

Liver-spleen scans can identify liver metastases and splenomegaly. Bone scans are sensitive in identifying metastases before they are evident on x-ray. Because a positive scan requires new bony formation (i.e.., osteoblastic activity), this technique is useless in neoplasms that are purely lytic (e.g., multiple myeloma); routine bone x-rays are the study of choice in such diseases. Gallium scans can help in staging lymphoid neoplasms. Radiolabeled monoclonal antibodies (e.g., to carcinoembryonic antigen, small cell lung cancer cells) provide important staging data in various neoplasms (e.g., colon cancer, small cell lung cancer). See The Merck Manual of Diagnosis & Therapy, Beers & Brakow, 17th edition, Published by Merck Research Labs, Sec. 11, Chapter 84, Hematology and Oncology, Overview of Cancer (1999).

It is preferred that the methods and compositions of the present invention are used in the treatment and/or prevention of a neoplasia or neoplasia-related disorder in a subject that is suffering from or is predisposed to a neoplasia or neoplasia-related disorder.

As used herein, the term “subject” for purposes of treatment includes any subject, and preferably is a subject who is in need of the treatment of neoplasia, or who needs treatment of a neoplasia-related disorder. For purposes of prevention, the subject is any subject, and preferably is a subject that is at risk for, or is predisposed to, developing a neoplasia or neoplasia-related disorder.

In preferred embodiments, the subject is one that is in need of prevention or treatment of a neoplasia or neoplasia-related disorder.

As used herein, the term “subject in need of” refers to any subject who is suffering from or is predisposed to neoplasia or any neoplasia-related disorder described herein. The terms “subject in need of” also refer to any subject that requires a lower dose of conventional neoplasia treatment agents. In addition, the terms “subject in need of” means any subject who requires a reduction in the side-effects of a conventional treatment agent. Furthermore, the terms “subject in need of” means any subject who requires improved tolerability to any conventional treatment agent for a neoplasia disorder therapy.

As used herein, the terms “predisposed to” and “at risk for,” both of which are used interchangeably herein, mean any subject at risk for developing neoplasia or at risk for re-developing neoplasia during a relapse of such a disorder. For example, after treatment, many neoplasia disorders subside into remission, meaning that the disease is present, but inactive within the subject and is thus, capable of re-developing at a later time. The subject may be at risk due to genetic predisposition, diet, lifestyle, age, exposure to radiation, exposure to neoplasia-causing agents, and the like.

The methods and compositions of the present invention not only encompass the prevention or treatment of neoplasia and neoplasia-related disorders in humans, but also in several animals. For example, many animals also suffer adverse consequences related to neoplasia. Moreover, many neoplasia and neoplasia-related disorders in dogs respond to the same treatment used in humans. Accordingly, besides being useful for humans, the methods and compositions of the present invention also encompass the treatment and prevention of neoplasia and neoplasia-related disorders in other mammals, including horses, dogs, cats, rats, mice, sheep, pigs, cattle, hamsters, gerbils, and the like. Thus, it is preferred that the subject is an animal, and yet more preferred, the subject is a mammal. Preferably, the mammal is a human.

A therapy comprising a Cox-2 inhibitor in combination with a DNA topoisomerase I inhibitor encompasses the treatment and prevention of such neoplasia or neoplasia-related disorder symptoms in a subject suffering from such symptoms.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

The term “neoplasia” or “neoplasia disorder” also includes any complications that arise from having such a disorder. Thus, the term “neoplasia-related disorder” includes any subsequent disease, disorder, injury or condition that may arise from having a neoplasia. The term “neoplasia-related disorder” refers to any condition where developing a neoplasia is a risk factor for developing health complications.

The methods and compositions of the present invention may be used for the treatment or prevention of several neoplasia disorders and neoplasia-related disorders including, but are not limited to, acral lentiginous melanoma, actinic keratosis, adenocarcinoma, adenoid cystic carcinoma, adenoma, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumor, breast cancer, bronchial gland carcinoma, capillary carcinoma, carcinoids, carcinoma, carcinosarcoma, cavernous cell carcinoma, central nervous system lymphoma, cerebral astrocytoma, childhood cancers, cholangiocarcinoma, chondrosarcoma, chorioid plexus papilloma and carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal cancer, epithelioid carcinoma, esophageal cancer, Ewing's sarcoma, extragonadal germ cell tumor, fibrolamellar carcinoma, focal nodular hyperplasia, gallbladder cancer, gastrinoma, germ cell tumors, gestational trophoblastic tumor, glioblastoma, glioma, glucagonoma, hemangioblastoma, hemangioendothelioma, hemangioma, hepatic adenoma, hepatic adenomatosis, hepatocellular carcinoma, Hodgkin's lymphoma, hypopharyngeal cancer, hypothalamic and visual pathway glioma, insulinoma, interepithelial squamous cell neoplasia, intraepithelial neoplasia, intraocular melanoma, invasive squamous cell carcinoma, islet cell carcinoma, Kaposi's sarcoma, kidney cancer, large cell carcinoma, laryngeal cancer, leiomyosarcoma, lentigo maligna melanoma, leukemia-related disorders, lip and oral cavity cancer, liver cancer, lung cancer, lymphoma, malignant mesothelial tumors, malignant thymoma, medulloblastoma, medulloepithelioma, melanoma, meningeal carcinoma, merkel cell carcinoma, mesothelial carcinoma, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma, nodular melanoma, non-Hodgkin's lymphoma, oat cell carcinoma, oligodendroglial carcinoma, oral cancer, oropharyngeal cancer, osteosarcoma, ovarian cancer, ovarian germ cell tumor, pancreatic cancer, papillary serous adenocarcinoma, parathyroid cancer, penile cancer, pheochromocytoma, pineal and supratentorial primitive neuroectodermal tumors, pineal cell carcinoma, pituitary tumors, plasma cell neoplasm, plasmacytoma, pleuropulmonary blastoma, prostate cancer, pseudosarcoma, pulmonary blastoma, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous cell carcinoma, submesothelial carcinoma, superficial spreading melanoma, thyroid cancer, undifferentiated carcinoma, urethral cancer, uterine sarcoma, uveal melanoma, vaginal cancer, verrucous carcinoma, vipoma, vulvar cancer, Waldenstrom's macroglobulinemia, well differentiated carcinoma, and Wilm's tumor.

In other preferred embodiments, the present invention encompasses a kit for preventing or treating neoplasia and neoplasia-related disorders in a subject that is in need of such prevention or treatment, the kit comprising one dosage form comprising a Cox-2 inhibitor and a second dosage form comprising a DNA topoisomerase I inhibitor.

The following examples describe embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples. In the examples, all percentages are given on a weight basis unless otherwise indicated.

### EXAMPLE 1

This example shows the preparation of celecoxib.

Step 1: Preparation of 1-(4-methylphenyl)-4,4,4-trifluorobutane-1,3-dione. Following the disclosure provided in U.S. Pat. No. 5,760,068, 4′-methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide. To the dione from Step 1 (4.14 g, 18.0 mmol) in 75 mL absolute ethanol, 4.26 g (19.0 mmol) 4-sulphonamido-phenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄N₃O₂SF₃; C, 53.54; H, 3.70; N, 11.02. The composition that was found by analysis was: C, 53.17; H, 3.81; N, 10.90.

### EXAMPLE 2

This example illustrates the production of a composition containing celecoxib and a indenoisoquinoline DNA topoisomerase 1 inhibitor, such as 6-(3-carboxy-1-propyl)-5,6-dihydro-5,11-diketo-11H-indeno[1,2-c]isoquinoline, and a pharmaceutical composition containing the combination.

Following the disclosure of U.S. Pat. No. 6,509,344, 6-(3-carboxy-1-propyl)-5,6-dihydro-5,11-diketo-11H-indeno[1,2-c]isoquinoline can be prepared by the following procedure:

Step 1: Preparation of 5,6-Dihydro-6-(4-hydroxy-1-butyl)-5,11-diketo-11H-indeno[1,2-c]isoquinoline. 4-Amino-1-butanol (0.891 g, 10 mmol) was added to a chloroform (30 mL) solution of benz[d]indeno[1,2-b]pyran-5,11-dione (2.48 g, 10 mmol) and the reaction mixture was stirred at room temperature 2 days. The reaction mixture turned dark red. The reaction mixture was taken in chloroform (100 mL) and washed with water (2×50 mL), 0.5 N HCl (50 mL), brine (100 mL) and dried (Na₂SO₄) and concentrated to give the crude product. The product was filtered through a short column of silica gel and the polar fraction concentrated to afford a reddish brown solid which was crystallized from isopropanol to yield the product (2.56 g, 80%).

Step 2: Preparation of 6-(3-carboxy-1-propyl)-5,6-dihydro-5,11-diketo-11H-indeno[1,2-c]isoquinoline. The indenoisoquinoline from Step 1 (0.319 g, 1 mmol) was dissolved in acetone (50 mL) and cooled in an ice bath. Jones reagent was added dropwise to the cold solution of the alcohol until the red color of the reagent persisted. The excess Jones reagent was quenched by adding few drops of isopropyl alcohol. The reaction mixture was filtered through a small pad of celite and the residue was washed with acetone (50 mL). The combined filtrate was concentrated and the residue was dissolved in saturated bicarbonate (100 mL) and the aqueous layer was washed with chloroform (2×30 mL). The aqueous layer was neutralized with concd HCl and extracted in CHCl.sub.3 (3×50 mL). The combined organic layer was dried (Na₂SO₄) and concentrated to afford the acid as an orange solid. The solid was crystallized from isopropyl alcohol to yield orange crystals (0.320 g, 96%).

Celecoxib can be prepared as described in Example 1, or can be obtained under the trade name CELEBREX® from Pharmacia Corporation, Peapack, N.J.

A therapeutic composition of the present invention can be formed by intermixing 6-(3-carboxy-1-propyl)-5,6-dihydro-5,11-diketo-11H-indeno[1,2-c]isoquinoline, 50 g; and 4-[5-(4-methylphenyl)-3-(trifluoro-methyl)-1H-pyrazol-1-yl]benzenesulfonamide (200 g, as produced in Example 1, or as available from Pharmacia Corporation, Peapack, N.J., under the tradename CELEBREX®), in a suspension or solution with a sterile pharmaceutically acceptable liquid.

After mixing, the combination of 6-(3-carboxy-1-propyl)-5,6-dihydro-5,11-diketo-11H-indeno[1,2-c]isoquinoline and celecoxib forms a therapeutic composition that is sufficient for the production of about 1000 human single dose units. Each single dose unit contains about 50 mg of 6-(3-carboxy-1-propyl)-5,6-dihydro-5,11-diketo-11H-indeno[1,2-c]isoquinoline and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule can contain about the same amount of the active ingredients as each of the single dose units of the liquid preparation described above.

Therapeutic and pharmaceutical compositions comprising a combination of any of the Cox-2 selective inhibitors and any of the sources of DNA topoisomerase I inhibitory agents that are described above can be formed by similar methods.

### EXAMPLE 3

This example illustrates an animal model that can be used for in vivo analysis of the efficacy of a Cox-2 inhibitor in combination with a DNA topoisomerase I inhibitor in the treatment of neoplasia.

In vivo assessment of the effect of a pharmaceutical composition comprising a Cox-2 inhibitor and a DNA topoisomerase I inhibitor (hereafter Cox-2/topo-I) on proliferation of certain cancer cells can be performed using a nude mouse xenograft model. Mice can be chosen from several commercially available athymic strains including BALB/c nu/nu, C57BL/6 nulnu, NIH-III nu/nu, or any other strain of nude mouse known to one of skill in the art. Nulnu mice from 4-8 weeks of age can be injected subcutaneously in one flank with a predetermined number of tumor cells (e.g. 3×10⁶ cells). Tumor cells used for injection can be any cancerous cell, for example, any of the several hundred cancer cell lines available from the American Type Culture Collection (Rockville, Md.), including, for example, PC-3 (human prostate adenocarcinoma), HT-3 (human cervical carcinoma), NCI-H1417 (human small cell lung carcinoma), SaOS (human osteosarcoma), and HCC38 (human primary ductal breast carcinoma). The dose of cells utilized in the injection should be sufficient to produce palpable tumors within about 7 days. Animals will be examined daily for evidence of tumor growth, and treatment will be initiated when tumors of a predetermined size (e.g. 0.5 cm in diameter) are identified.

Cox-2/topo-I will be administered on a daily basis for a set period of time from detection of the appropriate sized tumor (day 1), with control animals receiving vehicle alone. Administration can be subcutaneous, intraperitoneal, via tail vein injection, or by any means known to one of skill in the art. Groups of animals will each be treated with different Cox-2/topo-I doses, and the control animals will be administered vehicle alone. Tumor dimensions will be measured every other day using vernier calipers. Tumor volume (mm³) can be calculated as length×(width)²/2. Relative tumor volume (RTV) can be calculated as RTV=tumor volume on day x/tumor volume on day 1. Comparisons between volumes at various time points, and between experimental and control animals, can be made using standard statistical methods known to those with skill in the art, such as the student's t test. Animals will be sacrificed when maximum tumor diameter in any plane is 1.5 cm. Animals that survive to 28 days without requiring sacrifice can be retreated with additional cycles of therapy.

Assessment of Tumor Regression in a Nude Mouse Model.

To determine whether Cox-2/topo-I is capable of inducing regression of large, established tumors, tumor cells can be implanted as described above and allowed to grow until the maximal tumor diameter exceeds 1 cm. Animals can be treated with a dose of Cox-2/topo-I for a predetermined number of days, optionally repeating in cycles. In the absence of treatment, such animals invariably require sacrifice within 1-5 days. Tumor volumes can be recorded as noted above. It is believed that mouse morbidity and/or tumor growth will be reduced in the animals receiving Cox-2/topo-I treatment in comparison with the animals receiving no treatment or receiving vehicle alone.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

1. A method for preventing or treating neoplasia or a neoplasia-related disorder in a subject, the method comprising administering in combination therapy to the subject a Cox-2 selective inhibitor and a DNA topoisomerase I inhibitor; wherein the DNA topoisomerase I inhibitor comprises at least one agent selected from the group consisting of camptothecin in combination with poly-(l-glutamic acid), camptothecin in combination with NU1025, XR-11612, DX-8915f, anthracycline aclacinomycin A, harmane, harmine, harmaline, bulgarein, rebeccamycin, rebeccamycin R-3, luteolin, diospyrin, ecteinascidin 743, Ho-33342, Ho-33258, idarubicin, SN-38 in combination with 5-FU in sequential drug administration with SN-38 first, 9-NC in combination with 5-FU, BN-80927, fagaronine, ethoxidine, nitidine, MJ-III-65, S2, J-107088, karenitecin, BNP-1100 in combination with ZD-1694, β-lapachone, intoplicine, TAN-1518A, plaquiloside, GI-14721 1, camptothecin in combination with 7-hydroxystaurosporine, indeneisoquinolines, heteroaromatic[a]phenazine carboxamide derivatives, covalent conjugates of topoisomerase I and topoisomerase II inhibitors, 7-substituted camptothecin derivatives, highly lipophilic camptothecin derivatives, hexacyclic camptothecin analogues, trisbenzimidazoles, benzo[a]phenazine-11-carboxamide derivatives, XR-5000, phenoxodiol, AHMA, (5Z,9Z)-5,9-hexadecadienoic acid, RFS2000, TAS-103, 7-ethyl-10-[4-(1-piperidyl)-1-piperidyl]carbonyloxy-camptothecin, disulfiram, isoaurostatin, 6-[3-(2-hydroxyethyl)aminopropyl]-5,6-dihydro-2,3-dimethoxy-8,9-methylenedioxy-5,11-dioxo-11H-indeno [1,2-c]isoquinoline hydrochloride, BNP1350 and ring-substituted 11-oxo-11H-indeno[1,2-b]quinoline-6-carboxamides.

2. The method of claim 1, wherein the Cox-2 selective inhibitor comprises at least one compound that is selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, MK-966, L-783003, T-614, D-1376, L-748731, CGP-28238, BF-389, GR-253035, prodrugs of any of them, and mixtures thereof.

3. The method of claim 1, wherein the Cox-2 selective inhibitor comprises celecoxib.

4. The method of claim 1, wherein the neoplasia or neoplasia-related disorder is selected from the group consisting of neoplasias of the lung, breast, skin, stomach, prostate, intestine, esophagus, bladder, head, neck, brain, cervix and ovary.

5. A method for preventing or treating neoplasia or a neoplasia-related disorder in a subject, the method comprising administering in combination therapy to the subject a Cox-2 selective inhibitor and a DNA topoisomerase I inhibitor, wherein the Cox-2 selective inhibitor comprises at least one compound of formulas (XXXVII) to (LI) herein.

6. The method of claim 5 wherein the DNA topoisomerase I inhibitor is irinotecan or a salt or prodrug thereof.

7. The method of claim 5, wherein the neoplasia or neoplasia-related disorder is selected from the group consisting of neoplasias of the lung, breast, skin, stomach, prostate, intestine, esophagus, bladder, head, neck, brain, cervix and ovary.
